(19) United States
(12) Patent Application Publication
(10) Pub. No.: US 2023/0391890 A1
(43) Pub. Date: Dec. 7, 2023

(54) THERAPEUTIC CEMIP ANTIBODIES

(71) Applicants: CORNELL UNIVERSITY, Ithaca, NY (US); TRI-INSTITUTIONAL THERAPEUTICS DISCOVERY INSTITUTE, New York, NY (US)

(72) Inventors: David C. LYDEN, New York, NY (US); Goncalo RODRIGUES, New York, NY (US); Irena RAJNPREHT, New York, NY (US); Abdul KHAN, New York, NY (US); Ivo LORENZ, New York, NY (US); Irina MATEI, New York, NY (US)

(21) Appl. No.: 18/249,187

(22) PCT Filed: Oct. 15, 2021
(86) PCT No.: PCT/US2021/055280

§ 371 Date: Apr. 14, 2023

Related U.S. Application Data
(60) Provisional application No. 63/092,388, filed on Oct. 15, 2020.

(51) Int. Cl. C07K 16/40; A61P 35/04

(52) U.S. Cl. CPC C07K 16/40; A61P 35/04; C07K 2317/76; C07K 2317/92

(57) ABSTRACT

The present disclosure relates to antibody-based molecules, including antibodies, epitope-binding domains thereof, and antibody derivative as described herein, that are capable of binding and inhibiting cell migration-inducing and hyaluronan-binding protein (CEMIP). Such antibody-based molecules are useful for the treatment of conditions where a subject is in need of blocking the activity of CEMIP.

This application claims priority benefit of U.S. Provisional Patent Application No. 63/092,388, filed Oct. 15, 2020, which is hereby incorporated by reference in its entirety.

This invention was made with government support under W81XWH-13-1-0427 awarded by the Department of Defense. The government has certain rights in the invention.

### FIELD

The present disclosure relates to antibody-based molecules, including full-length antibodies, epitope-binding domains thereof, and antibody derivatives that are capable of binding to and inhibiting cell migration-inducing and hyaluronan-binding protein (CEMIP). The present disclosure further discloses methods of treatment using the CEMIP antibodies.

### BACKGROUND

Despite advances in understanding the molecular determinants that drive metastasis, metastatic entry and adaptation to specific organs, particularly the brain, remain poorly understood. The incidence of brain metastasis (BrM) is ten-fold higher than all primary brain tumours combined (Maher et al., “Brain Metastasis: Opportunities in Basic and Translational Research,” Cancer Res 69:6015-6020 (2009)). Brain metastases most commonly arise from lung and breast cancer, have poor prognosis and high mortality, and lack effective therapy (Eichler et al., “The Biology of Brain Metastases-Translation to New Therapies,” Nat Rev Clin Oncol 8:344-356 (2011)). Hence, identifying tumour-intrinsic properties and/or drivers of the crosstalk between tumour cells and the brain microenvironment that can be targeted to prevent and/or treat BrM is critical.

Recently, the contributions of tumour-secreted exosomes to brain metastatic colonization were defined (Rodrigues et al., “Tumour Exosomal CEMIP Protein Promotes Cancer Cell Colonization in Brain Metastasis,” Nature Cell Biol. 21(11): 1403-1412 (2019)). Proteomic analysis identified cell migration-inducing and hyaluronan-binding protein (CEMIP) as elevated in exosomes from brain metastatic cells but not lung or bone metastatic cells (Rodrigues et al., Nature Cell Biol. 21(11): 1403-1412 (2019)). CEMIP depletion in tumour cells impaired brain metastasis, disrupting invasion and tumour cell association with the brain vasculature, phenotypes rescued by pre-conditioning the brain microenvironment with CEMIP+ exosomes (Rodrigues et al., Nature Cell Biol. 21(11): 1403-1412 (2019)). Moreover, uptake of CEMIP+ exosomes by brain endothelial and microglial cells induced endothelial cell branching and inflammation in the perivascular niche by upregulating the pro-inflammatory cytokines encoded by Ptgs2, Tnf and Ccl/Cxcl, known to promote brain vascular remodelling and metastasis (Rodrigues et al., Nature Cell Biol. 21(11): 1403-1412 (2019)). What is needed are clinically useful inhibitors of CEMIP that can be administered for the prevention and treatment of brain metastasis and other CEMIP conditions.

The present disclosure is directed to overcoming these and other limitations in the art.

### SUMMARY

The present disclosure relates to antibody-based molecules, including antibodies, epitope-binding domains thereof, and antibody derivative as described herein, that are capable of binding and inhibiting cell migration-inducing and hyaluronan-binding protein (CEMIP). Such antibody-based molecules are useful for the treatment of conditions where a subject is in need of blocking the activity of CEMIP.

A first aspect of the disclosure is directed to an antibody-based molecule that binds to CEMIP and comprises a heavy chain variable region, where the heavy chain variable region comprises: (i) a complementarity-determining region 1 (CDR-H1) comprising an amino acid sequence of any one of SEQ ID NOs: 2-8 or a modified amino acid sequence of any one of SEQ ID NOs: 2-8, said modified sequence having at least 80% sequence identity to any one of SEQ ID NOs: 2-8; (ii) a complementarity-determining region 2 (CDR-H2) comprising an amino acid sequence of any one of SEQ ID NOs: 9-15 or a modified amino acid sequence of any one of SEQ ID NOs: 9-15, said modified sequences having at least 80% sequence identity to any one of SEQ ID NOs: 9-15; and (iii) a complementarity-determining region 3 (CDR-H3) comprising an amino acid sequence of any one of SEQ ID NOs: 16-22, or a modified amino acid sequence of any one of SEQ ID NO: 16-22, said modified sequence having at least 80% sequence identity to any one of SEQ ID NOs: 16-22.

The antibody-based molecule described herein may further comprise a light chain variable region, wherein said light chain variable region comprises: a complementarity-determining region 1 (CDR-L1) having an amino acid sequence of any one of SEQ ID NOs: 23-29, or a modified amino acid sequence of any one of SEQ ID NO: 23-29, said modified sequence having at least 80% sequence identity to any one of SEQ ID NO: 23-29; a complementarity-determining region 2 (CDR-L2) having an amino acid sequence of any one of SEQ ID NOs: 30-36, or a modified amino acid sequence of any one of SEQ ID NO: 30-36, said modified sequence having at least 80% sequence identity to any one of SEQ ID NO: 30-36; and a complementarity-determining region 3 (CDR-L3) having an amino acid sequence of any one of SEQ ID NOs: 37-43, or a modified amino acid sequence of any one of SEQ ID NO: 37-43, said modified sequence having at least 80% sequence identity to any one of SEQ ID NO: 37-43.

Another aspect of the disclosure is directed to an isolated polynucleotide encoding the CEMIP antibody-based molecule as described herein.

Another embodiment of the disclosure is directed to a vector comprising at least one polynucleotide encoding the CEMIP antibody-based molecule as described herein.

Another aspect of the disclosure is directed to a pharmaceutical composition comprising the CEMIP antibody-based molecule as described herein, a polynucleotide encoding the CEMIP antibody-based molecule described herein, or a vector comprising at least one polynucleotide encoding the CEMIP antibody-based molecule as described herein; and a pharmaceutically acceptable carrier.

Another aspect of the disclosure is directed to a method of inhibiting cell migration-inducing and hyaluronan-binding protein (CEMIP) signaling in a subject. This method involves administering to the subject the pharmaceutical composition as described herein, wherein the pharmaceutical composition is administered in an amount effective to decrease CEMIP signaling in the subject relative to CEMIP signaling in the subject prior to said administering.

Another aspect of the present disclosure is directed to a method of treating or inhibiting brain metastasis in a subject. This method involves administering, to a subject having a primary tumor, a cell migration-inducing and hyaluronan-binding protein (CEMIP) antibody or binding fragment thereof as described herein in an amount effect to treat or prevent brain metastasis in the subject.

Another aspect of the present disclosure is directed to a method of treating an autoimmune condition in a subject. This method involves administering, to a subject having an autoimmune condition, the pharmaceutical composition as described herein, thereby treating the autoimmune condition in the subject.

Another aspect of the present disclosure is directed to a method of treating an inflammatory condition in a subject. This method involves administering, to a subject having an inflammatory condition, the pharmaceutical composition of claim 20, thereby treating the inflammatory condition in the subject.

### BRIEF DESCRIPTION OF THE DRAWINGS

FIG. 1 shows binding of anti-KIAA1199/CEMIP monoclonal antibodies to recombinant protein. Mice were immunized with plasmids containing full length human CEMIP DNA. B cells were isolated from the spleens of immunized mice, and hybridomas were generated. Hybridoma supernatants were screened by ELISA for binding to recombinant KIAA1199/CEMIP protein. Hybridoma from positive hits were subcloned, followed by purification of the monoclonal antibodies and analysis by ELISA using recombinant KIAA1199/CEMIP protein.

FIG. 2 shows validation of anti-CEMIP/KIAA1199 binding antibodies by flow cytometry. The binding of anti-CEMIP antibodies to native CEMIP expressed on the surface of human MKN45 gastric and N2LA lung cancer cell lines is shown. Two micrograms of indicated antibodies were used to stain 2×10⁵ cells and binding was revealed with an Ax647-labelled goat anti-mouse secondary antibody (Biolegend).

FIG. 3 shows a second phase flow cytometry-based screen for anti-KIAA1199/CEMIP antibodies. Binding of neat exhausted hybridoma supernatants to 2×10⁵ MKN45 cells was detected with an Ax647-labelled goat anti-mouse secondary antibody (Biolegend) using flow cytometry. The y-axis indicates the hybridoma clone name, and the x-axis represents the median fluorescence intensity for the Ax647-labelled goat anti-mouse secondary antibody (Biolegend). Coloured bars indicate hybridoma supernatants that bound above the positive hybridoma control level (10F01B02).

FIG. 4 shows second phase ELISA screen for anti-KIAA1199/CEMIP monoclonal antibodies. Mice were immunized with plasmids containing full length human CEMIP DNA. B cells were isolated from the spleens of immunized mice, and hybridomas were generated and selected from semi-solid media. Clonal hybridoma supernatants were screened by ELISA for binding to recombinant KIAA1199/CEMIP protein. Monoclonal antibodies were purified from the supernatant of hybridoma and analyzed by ELISA using recombinant KIAA1199/CEMIP protein.

FIG. 5 shows validation of CEMIP/KIAA1199 loss in MKN45 CRISPR/Cas9 clones by flow cytometry. The binding of anti-CEMIP antibodies to native CEMIP expressed on the surface of wild-type and CEMIP/KIAA1199 CRISPR/Cas9 human MKN45 gastric cell lines is shown. Two micrograms of 10F01B02 anti-CEMIP/KIAA1199 antibody were used to stain 2×10⁵ cells and binding was revealed with an Ax647-labelled goat anti-mouse secondary antibody (Biolegend). This experiment also demonstrates the specificity of the anti-CEMIP/KIAA1199 antibody.

FIG. 6 shows the binding specificity of antibody clones 01F11A01 (cAb4855), 07F11C02 (cAb4853), and 10F01B02 (cAb4854) for CEMIP/KIAA1199. The binding of anti-CEMIP antibodies to native CEMIP expressed on the surface of wild-type and CEMIP/KIAA1199 independent knock-out clones (KO6 and KO11) generated via CRISPR/Cas9 technology in the human MKN45 gastric cell lines. The flow cytometry data for each clone shows robust binding to the wild-type-MKN45 cells, but not the CEMIP KO clones.

FIGS. 7A-7B show inhibition of CEMIP mediated metastasis in a 3D organotypic brain slice assay. FIG. 7A provides a schematic overview of the 3D organotypic brain slice assay and FIG. 7B is a graph showing CEMIP antibody mediated inhibition of GFP-labelled parental MDA-MB231 breast cancer cell growth in the brain microenvironment.

### DETAILED DESCRIPTION

The present disclosure relates to antibody-based molecules, including antibodies, epitope-binding domains thereof, and antibody derivative as described herein, that are capable of binding and inhibiting cell migration-inducing and hyaluronan-binding protein (CEMIP). Such antibody-based molecules are useful for the treatment of conditions where a subject is in need of blocking the activity of CEMIP.

A first aspect of the present disclosure is directed to an antibody-based molecule that binds an epitope of CEMIP. CEMIP, also known as KIAA1199, is a Wnt-related protein known for mediating depolymerization of hyaluronic acid via the cell membrane-associated clathrin-coated pit endocytic pathway. As shown herein, CEMIP is enriched in brain metastatic breast and lung tumor derived exosomes and promotes brain metastasis by generating a pro-metastatic environment. The nucleotide sequence encoding CEMIP is known in the art, see e.g., UniProtKB Accession No. Q8WUJ3. The amino acid sequence of CEMIP is provided below as SEQ ID NO: 1

SEQ ID NO: 1

```
                10 20 30 40
MGAAGRQDEL FKAMLTISWL TLICFPGATS TVAAGCPDQS
                50 60 70 80
PELQPWNPGH DQDHHVHIGQ GKILLLISSA TVYSIHISEG
                90 100 110 120
GKIVIKDHDE PIVERTRHIL IDNGGELHAG SALCPFQGNE
              130 140 150 160
TIILYGRADE GIQPDPYYGL KYIGVGKGGA LELHGQKKLS
              170 180 190 200
WTFLNKTIHP GGMAEGGYFF ERSWGHRGVI VHVIDPKSGT
              210 220 230 240
VIHSDREDTY RSKKESERLV QYLNAVPDGR ILSVAVNDEG
              250 260 270 280
SRNLDDMARK AMTKLGSKHF IHLGFRHPWS FLTVKGNPSS
              290 300 310 320
SVEDHIEYHG HRGSAAARVE KLFQTEHGEY FNVSLSSEWV
              330 340 350 360
QDVEWIEWFD HDKVSQTKGG EKISDLWKAH PGKICNRPID
              370 380 390 400
IQATTMDGVN LSTEVVYKKG QDYRFACYDR GRACRSYRVR
              410 420 430 440
FLCGKPVRPK ITVTIDINVN STILNLEDNV QSWKPGDTLV
              450 460 470 480
IASTDYSMYQ AEEFQVLPCR SCAPNQVKVA GKPMYLHIGE
              490 500 510 520
EIDGVDMRAE VGLLSRNIIV MGEMEDKCYP YRNHICNFFD
              530 540 550 560
FDTFGGHIKF ALGFKAAHLE GTELKHMGQQ LVGQYPIHFH
              570 580 590 600
LAGDVDERGG YDPPTYIRDL SIHHTFSRCV TVHGSNGLLI
              610 620 630 640
KDVVGYNSLG HCFFTEDGPE ERNTFDHCIG LIVKSGTLLP
              650 660 670 680
SDRDSKMCKM ITEDSYPGYI PKPRQDCNAV STFWMANPNN
              690 700 710 720
NLINCAAAGS EETGFWFIFH HVPTGPSVGM YSPGYSEHIP
              730 740 750 760
LGKFYNNRAH SNYRAGMIID NGVKTTEASA KDKRPFLSII
              770 780 790 800
SARYSPHQDA DPLKPREPAI IRHFIAYKNQ DHGAWLRGGD
              810 820 830 840
VWLDSCREAD NGIGLTLASG GIFPYDDGSK QEIKNSLFVG
              850 860 870 880
ESGNVGTEMM DNRIWGPGGL DHSGRTLPIG QNFPIRGIQL
              890 900 910 920
YDGPINIQNC TFRKEVALEG RHTSALAFRL NNAWQSCPHN
              930 940 950 960
NVIGIAFEDV PITSRVFFGE PGPWFNQLDM DGDKTSVEHD
              970 980 990 1000
VDGSVSEYPG SYLTKNDNWL VRHPDCINVP DWRGAICSGC
            1010 1020 1030 1040
YAQMYIQAYK TSNLRMKIIK NDFPSHPLYL EGALTRSTHY
            1050 1060 1070 1080
QQYQPVVTLQ KGYTIHWDQT APAELAIWLI NENKGDWIRV
            1090 1100 1110 1120
GLCYPRGTTF SILSDVHNRL LKQTSKTGVF VRTLQMDKVE
            1130 1140 1150 1160
QSYPGRSHYY WDEDSGLLFL KLKAQNEREK FAFCSMKGCE
            1170 1180 1190 1200
RIKIKALIPK NAGVSDCTAT AYPKFTERAV VDVPMPKKLE
            1210 1220 1230 1240
GSQLKIKDHF LEVKMESSKQ HFFHLWNDFA YIEVDGKKYP
            1250 1260 1270 1280
SSEDGIQVVV IDGNQGRVVS HTSERNSILQ GIPWQLFNYV
            1290 1300 1310 1320
ATIPDNSIVL MASKGRYVSR GPWTRVLEKL GADRGLKLKE
            1330 1340 1350 1360
QMAFVGFKGS FRPIWVILDT EDHKAKIFQV VPIPVVKKKK
L
```

Suitable CEMIP antibody-based molecules of the present disclosure include, without limitation full antibodies, epitope binding fragments of whole antibodies, and antibody derivatives. An epitope binding fragment of an antibody can be obtained through the actual fragmenting of a parental antibody (for example, a Fab or (Fab)₂ fragment). Alternatively, the epitope binding fragment is an amino acid sequence that comprises a portion of the amino acid sequence of such parental antibody. As used herein, a molecule is said to be a “derivative” of an antibody (or relevant portion thereof) if it is obtained through the actual chemical modification of a parent antibody or portion thereof, or if it comprises an amino acid sequence that is substantially similar to the amino acid sequence of such parental antibody or relevant portion thereof (for example, differing by less than 30%, less than 20%, less than 10%, or less than 5% from such parental molecule or such relevant portion thereof, or by 10 amino acid residues, or by fewer than 10, 9, 8, 7, 6, 5, 4, 3 or 2 amino acid residues from such parental molecule or relevant portion thereof).

An antibody of the present disclosure is an intact immunoglobulin as well as a molecule having an epitope-binding fragment thereof. As used herein, the terms “fragment”, “region”, and “domain” are generally intended to be synonymous, unless the context of their use indicates otherwise. Naturally occurring antibodies typically comprise a tetramer which is usually composed of at least two heavy (H) chains and at least two light (L) chains. Each heavy chain is comprised of a heavy chain variable (V_{H}) region and a heavy chain constant (C_{H}) region, usually comprised of three domains (C_{H}1, C_{H}2 and C_{H}3 domains). Heavy chains can be of any isotype, including IgG (IgG1, IgG2, IgG3 and IgG4 subtypes), IgA (IgA1 and IgA2 subtypes), IgM and IgE. Each light chain is comprised of a light chain variable (V_{L}) region and a light chain constant (C_{L}) region. Light chains include kappa chains and lambda chains. The heavy and light chain variable regions are typically responsible for antigen recognition, while the heavy and light chain constant regions may mediate the binding of the immunoglobulin to host tissues or factors, including various cells of the immune system (e.g., effector cells) and the first component (C1q) of the classical complement system. The V_{H} and V_{L} regions can be further subdivided into regions of hypervariability, termed “complementarity determining regions,” or “CDRs,” that are interspersed with regions of more conserved sequence, termed “framework regions” (FR). Each V_{H} and V_{L} region is composed of three CDR domains and four FR domains arranged from amino-terminus to carboxy-terminus in the following order: FR1-CDR1-FR2-CDR2-FR3-CDR3-FR4. The variable regions of the heavy and light chains contain a binding domain that interacts with an antigen. Of particular relevance are antibodies and their epitope-binding fragments that have been “isolated” so as to exist in a physical milieu distinct from that in which it may occur in nature or that have been modified so as to differ from a naturally-occurring antibody in amino acid sequence.

Fragments of antibodies (including Fab and (Fab)₂ fragments) that exhibit epitope-binding ability can be obtained, for example, by protease cleavage of intact antibodies. Examples of the CEMIP epitope-binding fragments encompassed within the present disclosure include (i) Fab′ or Fab fragments, which are monovalent fragments containing the V_{L}, V_{H}, C_{L} and C_{H}1 domains; (ii) F(ab′)₂ fragments, which are bivalent fragments comprising two Fab fragments linked by a disulfide bridge at the hinge region; (iii) Fd fragments consisting essentially of the V_{H} and C_{H}1 domains; (iv) Fv fragments consisting essentially of a V_{L} and V_{H} domain, (v) dAb fragments (Ward et al. “Binding Activities Of A Repertoire Of Single Immunoglobulin Variable Domains Secreted From Escherichia coli,” Nature 341:544-546 (1989) which is hereby incorporated by reference in its entirety), which consist essentially of a V_{H} or V_{L} domain and also called domain antibodies (Holt et al. “Domain Antibodies: Proteins For Therapy,” Trends Biotechnol. 21(11):484-490 (2003), which is hereby incorporated by reference in its entirety); (vi) camelid or nanobodies (Revets et al. “Nanobodies As Novel Agents For Cancer Therapy,” Expert Opin. Biol. Ther. 5(1):111-124 (2005), which is hereby incorporated by reference in its entirety), and (vii) isolated complementarity determining regions (CDR). An epitope-binding fragment may contain 1, 2, 3, 4, 5 or all 6 of the CDR domains of such antibody.

Such antibody fragments are obtained using conventional techniques known to those of skill in the art. For example, F(ab′)₂ fragments may be generated by treating a full-length CEMIP antibody with pepsin. The resulting F(ab′)₂ fragment may be treated to reduce disulfide bridges to produce Fab′ fragments. Fab fragments may be obtained by treating an IgG CEMIP antibody with papain and Fab′ fragments may be obtained with pepsin digestion of IgG a CEMIP antibody. A Fab′ fragment may be obtained by treating an F(ab′)₂ fragment with a reducing agent, such as dithiothreitol. Antibody fragments may also be generated by expression of nucleic acids encoding such fragments in recombinant cells (see e.g., Evans et al. “Rapid Expression Of An Anti-Human C5 Chimeric Fab Utilizing A Vector That Replicates In COS And 293 Cells,” J. Immunol. Meth. 184:123-38 (1995), which is hereby incorporated by reference in its entirety). For example, a chimeric gene encoding a portion of a F(ab′)₂ fragment could include DNA sequences encoding the CH1 domain and hinge region of the heavy chain, followed by a translational stop codon to yield such a truncated antibody fragment molecule. Suitable fragments capable of binding to a desired epitope may be readily screened for utility in the same manner as an intact antibody.

As referred to herein, “antibody derivatives” include those molecules that contain at least one epitope-binding domain of a CEMIP antibody, and are typically formed using recombinant techniques. One exemplary antibody derivative includes a single chain Fv (scFv). A scFv is formed from the two domains of the Fv fragment, the V_{L} region and the V_{H} region, which are encoded by separate gene. Such gene sequences or their encoding cDNA are joined, using recombinant methods, by a flexible linker (typically of about 10, 12, 15 or more amino acid residues) that enables them to be made as a single protein chain in which the V_{L} and V_{H} regions associate to form monovalent epitope-binding molecules (see e.g., Bird et al. “Single-Chain Antigen-Binding Proteins,” Science 242:423-426 (1988); and Huston et al. “Protein Engineering Of Antibody Binding Sites: Recovery Of Specific Activity In An Anti-Digoxin Single-Chain Fv Analogue Produced In Escherichia coli,” Proc. Natl. Acad. Sci. (U.S.A.) 85:5879-5883 (1988), which are hereby incorporated by reference in their entirety). Alternatively, by employing a flexible linker that is not too short (e.g., less than about 9 residues) to enable the V_{L} and V_{H} regions of a different single polypeptide chains to associate together, one can form a bispecific antibody, having binding specificity for two different epitopes.

In another embodiment, the CEMIP antibody derivative of the present disclosure is a divalent or bivalent single-chain variable fragment, engineered by linking two scFvs together either in tandem (i.e., tandem scFv), or such that they dimerize to form diabodies (Holliger et al. “‘Diabodies’: Small Bivalent And Bispecific Antibody Fragments,” Proc. Natl. Acad. Sci. (U.S.A.) 90(14), 6444-8 (1993), which is hereby incorporated by reference in its entirety). In yet another embodiment, the antibody is a trivalent single chain variable fragment, engineered by linking three scFvs together, either in tandem or in a trimer formation to form triabodies. In another embodiment, the antibody is a tetrabody single chain variable fragment. In another embodiment, the antibody is a “linear antibody” which is an antibody comprising a pair of tandem Fd segments (V_{H}-C_{H}1-V_{H}-C_{H}1) that form a pair of antigen binding regions (see Zapata et al. Protein Eng. 8(10):1057-1062 (1995), which is hereby incorporated by reference in its entirety). In another embodiment, the antibody derivative is a minibody, consisting of the single-chain Fv regions coupled to the C_{H}3 region (i.e., scFv-C_{H}3).

These and other useful CEMIP antibody fragments and derivative in the context of the present disclosure are discussed further herein. It also should be understood that the term antibody-based molecule, unless specified otherwise, also includes antibody-like polypeptides, such as chimeric antibodies and humanized antibodies, and antibody fragments retaining the ability to specifically bind to the antigen (epitope-binding fragments) provided by any known technique, such as enzymatic cleavage, peptide synthesis, and recombinant techniques.

A CEMIP antibody as generated herein may be of any isotype. As used herein, “isotype” refers to the immunoglobulin class (for instance IgG1, IgG2, IgG3, IgG4, IgD, IgA, IgE, or IgM) that is encoded by heavy chain constant region genes. The choice of isotype typically will be guided by the desired effector functions, such as antibody-dependent cellular cytotoxicity (ADCC) induction. Exemplary isotypes are IgG1, IgG2, IgG3, and IgG4. Either of the human light chain constant regions, kappa or lambda, may be used. If desired, the class of a CEMIP antibody of the present disclosure may be switched by known methods. For example, an antibody of the present disclosure that was originally IgM may be class switched to an IgG antibody of the present disclosure. Further, class switching techniques may be used to convert one IgG subclass to another, for instance from IgG1 to IgG2. Thus, the effector function of the antibodies of the present disclosure may be changed by isotype switching to, e.g., an IgG1, IgG2, IgG3, IgG4, IgD, IgA, IgE, or IgM antibody for various therapeutic uses.

In one embodiment, the CEMIP antibody-based molecules of the present disclosure are “humanized,” particularly if they are to be employed for therapeutic purposes. The term “humanized” refers to a chimeric molecule, generally prepared using recombinant techniques, having an antigen-binding site derived from an immunoglobulin from a non-human species and a remaining immunoglobulin structure based upon the structure and/or sequence of a human immunoglobulin. The antigen-binding site may comprise either complete non-human antibody variable domains fused to human constant domains, or only the complementarity determining regions (CDRs) as described herein of such variable domains grafted to appropriate human framework regions of human variable domains. The framework residues of such humanized molecules may be wild-type (e.g., fully human) or they may be modified to contain one or more amino acid substitutions not found in the human antibody whose sequence has served as the basis for humanization. Humanization lessens or eliminates the likelihood that a constant region of the molecule will act as an immunogen in human individuals, but the possibility of an immune response to the foreign variable region remains (LoBuglio, A. F. et al. “Mouse/Human Chimeric Monoclonal Antibody In Man: Kinetics And Immune Response,” Proc. Natl. Acad. Sci. USA 86:4220-4224 (1989), which is hereby incorporated by reference in its entirety). Another approach focuses not only on providing human-derived constant regions, but modifying the variable regions so as to reshape them as closely as possible to human form. The variable regions of both heavy and light chains contain three complementarity-determining regions (CDRs) which vary in response to the antigens in question and determine binding capability. The CDRs are flanked by four framework regions (FRs) which are relatively conserved in a given species and which putatively provide a scaffolding for the CDRs. Humanized antibodies of the present disclosure can thus be prepared by grafting the CDRs described herein derived from non-human antibody on the FRs present in a human antibody to be modified. Suitable methods for humanizing the non-human antibody described herein are known in the art see e.g., Sato, K. et al., Cancer Res 53:851-856 (1993); Riechmann, L. et al., “Reshaping Human Antibodies for Therapy,” Nature 332:323-327 (1988); Verhoeyen, M. et al., “Reshaping Human Antibodies: Grafting An Antilysozyme Activity,” Science 239:1534-1536 (1988); Kettleborough, C. A. et al., “Humanization Of A Mouse Monoclonal Antibody By CDR-Grafting: The Importance Of Framework Residues On Loop Conformation,” Protein Engineering 4:773-3783 (1991); Maeda, H. et al., “Construction Of Reshaped Human Antibodies With HIV-Neutralizing Activity,” Human Antibodies Hybridoma 2:124-134 (1991); Gorman, S. D. et al., “Reshaping A Therapeutic CD4 Antibody,” Proc. Natl. Acad. Sci. USA 88:4181-4185 (1991); Tempest, P. R. et al., “Reshaping A Human Monoclonal Antibody To Inhibit Human Respiratory Syncytial Virus Infection In Vivo,” Bio/Technology 9:266-271 (1991); Co, M. S. et al., “Humanized Antibodies For Antiviral Therapy,” Proc. Natl. Acad. Sci. USA 88:2869-2873 (1991); Carter, P. et al., “Humanization Of An Anti-p185her2 Antibody For Human Cancer Therapy,” Proc. Natl. Acad. Sci. USA 89:4285-4289 (1992); and Co, M. S. et al., “Chimeric And Humanized Antibodies With Specificity For The CD33 Antigen,” J. Immunol. 148:1149-1154 (1992), which are hereby incorporated by reference in their entirety. In any embodiment, humanized CEMIP antibodies of the present disclosure preserve all CDR sequences (for example, a humanized antibody containing all six CDRs from the mouse antibody). In other embodiments, humanized CEMIP antibodies of the present disclosure have one or more CDRs (one, two, three, four, five, six) which are altered with respect to the original antibody. Methods of humanizing an antibody are well-known in the art and suitable for humanizing the antibodies of the present disclosure (see, e.g., U.S. Pat. No. 5,225,539 to Winter; U.S. Pat. Nos. 5,530,101 and 5,585,089 to Queen and Selick; U.S. Pat. No. 5,859,205 to Robert et al.; U.S. Pat. No. 6,407,213 to Carter; and U.S. Pat. No. 6,881,557 to Foote, which are hereby incorporated by reference in their entirety).

Humanized CEMIP antibodies of the present disclosure encompass antibodies were only part of a CDR, namely the subset of CDR residues required for binding termed the “specificity determining residues” (“SDRs”) are incorporated into the humanized antibody. CDR residues not contacting antigen and not in the SDRs can be identified based on previous studies from regions of Kabat CDRs lying outside Chothia hypervariable loops (see, Kabat et al., SEQUENCES OF PROTEINS OF IMMUNOLOGICAL INTEREST, National Institutes of Health Publication No. 91-3242 (1992); Chothia, C. et al., “Canonical Structures For The Hypervariable Regions Of Immunoglobulins,” J. Mol. Biol. 196:901-917 (1987), which are hereby incorporated by reference in their entirety), by molecular modeling and/or empirically, or as described in Gonzales, N. R. et al., “SDR Grafting Of A Murine Antibody Using Multiple Human Germline Templates To Minimize Its Immunogenicity,” Mol. Immunol. 41:863-872 (2004), which is hereby incorporated by reference in its entirety. In such humanized antibodies, at positions in which one or more donor CDR residues is absent or in which an entire donor CDR is omitted, the amino acid occupying the position can be an amino acid occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of mouse amino acids in a humanized antibody and consequently decreasing potential immunogenicity. However, substitutions can also cause changes of affinity, and significant reductions in affinity are preferably avoided. Positions for substitution within CDRs and amino acids to substitute can also be selected empirically.

In accordance with the present disclosure, phage display technology can alternatively be used to increase (or decrease) CDR affinity of the CEMIP antibody-based molecules of the present disclosure. This technology, referred to as affinity maturation, employs mutagenesis or “CDR walking” and re-selection using the target antigen or an antigenic fragment thereof to identify antibodies having CDRs that bind with higher (or lower) affinity to the antigen when compared with the initial or parental antibody (see, e.g. Glaser et al., “Antibody Engineering By Codon-Based Mutagenesis In A Filamentous Phage Vector System,” J. Immunology 149:3903-3913 (1992), which is hereby incorporated by reference in its entirety). Mutagenizing entire codons rather than single nucleotides results in a semi-randomized repertoire of amino acid mutations. Libraries can be constructed consisting of a pool of variant clones each of which differs by a single amino acid alteration in a single CDR and which contain variants representing each possible amino acid substitution for each CDR residue. Mutants with increased (or decreased) binding affinity for the antigen can be screened by contacting the immobilized mutants with labeled antigen. Any screening method known in the art can be used to identify variant antibody-based binding molecules with increased or decreased affinity to the antigen (e.g., ELISA) (See Wu, H. et al., “Stepwise In Vitro Affinity Maturation Of Vitaxin, An Alphav Beta3-Specific Humanized mAb,” Proc. Natl. Acad. Sci. USA 95:6037-6042 (1998); Yelton et al., “Affinity Maturation Of The BR96 Anti-Carcinoma Antibody By Codon-Based Mutagenesis,” J Immunology 155:1994 (1995), which are hereby incorporated by reference in their entirety). CDR walking, which randomizes the light chain, may be used (see, Schier, R. et al., “Isolation Of Picomolar Affinity Anti-c-erbB-2 Single-Chain Fv By Molecular Evolution Of The Complementarity Determining Regions In The Center Of The Antibody Binding Site,” J. Mol. Biol. 263:551-567 (1996), which is hereby incorporated by reference in its entirety).

Methods for accomplishing such affinity maturation that are suitable for affinity maturation of the CEMIP antibody molecule disclosed herein are described, for example, in Krause, J. C. et al., “An Insertion Mutation That Distorts Antibody Binding Site Architecture Enhances Function of a Human Antibody,” MBio. 2(1): e00345-10 (2011); Kuan, C. T. et al., “Affinity-Matured Anti-Glycoprotein NMB Recombinant Immunotoxins Targeting Malignant Gliomas And Melanomas,” Int. J. Cancer 10.1002/ijc.25645 (2010); Hackel, B. J. et al., “Stability And CDR Composition Biases Enrich Binder Functionality Landscapes,” J Mol. Biol. 401(1):84-96 (2010); Montgomery, D. L. et al., “Affinity Maturation And Characterization Of A Human Monoclonal Antibody Against HIV-1 gp41,” MAbs 1(5):462-474 (2009); Gustchina, E. et al., “Affinity Maturation By Targeted Diversification Of The CDR-H2 Loop Of A Monoclonal Fab Derived From A Synthetic Naïve Human Antibody Library And Directed Against The Internal Trimeric Coiled-Coil Of Gp41 Yields A Set Of Fabs With Improved HIV-1 Neutralization Potency And Breadth,” Virology 393(1):112-119 (2009); Finlay, W. J. et al., “Affinity Maturation Of A Humanized Rat Antibody For Anti-RAGE Therapy: Comprehensive Mutagenesis Reveals A High Level Of Mutational Plasticity Both Inside And Outside The Complementarity-Determining Regions,” J. Mol. Biol. 388(3):541-558 (2009); Bostrom, J. et al., “Improving Antibody Binding Affinity And Specificity For Therapeutic Development,” Methods Mol. Biol. 525:353-376 (2009); Steidl, S. et al., “In Vitro Affinity Maturation Of Human GM-CSF Antibodies By Targeted CDR-Diversification,” Mot Immunol. 46(1):135-144 (2008); and Barderas, R. et al., “Affinity Maturation Of Antibodies Assisted By In Silico Modeling,” Proc. Natl. Acad. Sci. USA 105(26):9029-9034 (2008), which are hereby incorporated by reference in their entirety.

In one aspect of the present disclosure, the CEMIP-antibody-based molecule as described herein comprises the amino acid sequence of any one, any two, any three, any four, any five, or any six CDRs as provided in Tables 1 and 2 herein.

In one aspect, the antibody-based molecule that binds to CEMIP comprises a heavy chain variable region, where the heavy chain variable region comprises: (i) a complementarity-determining region 1 (CDR-H1) comprising an amino acid sequence of any one of SEQ ID NOs: 2-8 or a modified amino acid sequence of any one of SEQ ID NOs: 2-8, said modified sequence having at least 80%, at least 85%, at least 90%, at least 95%, or at least 98% sequence identity to any one of SEQ ID NOs: 2-8; (ii) a complementarity-determining region 2 (CDR-H2) comprising an amino acid sequence of any one of SEQ ID NOs: 9-15 or a modified amino acid sequence of any one of SEQ ID NOs: 9-15, said modified sequences having at least 80%, at least 85%, at least 90%, at least 95%, or at least 98% sequence identity to any one of SEQ ID NOs: 9-15; and (iii) a complementarity-determining region 3 (CDR-H3) comprising an amino acid sequence of any one of SEQ ID NOs: 16-22, or a modified amino acid sequence of any one of SEQ ID NO: 16-22, said modified sequence having at least 80%, at least 85%, at least 90%, at least 95%, or at least 98% sequence identity to any one of SEQ ID NOs: 16-22.

In any embodiment, the antibody-based molecule that binds to human CEMIP comprises a heavy chain variable region comprising the CDR-H1 of SEQ ID NO: 2, the CDR-H2 of SEQ ID NO: 9, and the CDR-H3 of SEQ ID NO: 16.

In any embodiment, the antibody-based molecule that binds to human CEMIP comprises a heavy chain variable region comprising the CDR-H1 of SEQ ID NO: 3, the CDR-H2 of SEQ ID NO: 10, and the CDR-H3 of SEQ ID NO: 17.

In any embodiment, the antibody-based molecule that binds to human CEMIP comprises a heavy chain variable region comprising the CDR-H1 of SEQ ID NO: 4, the CDR-H2 of SEQ ID NO: 11, and the CDR-H3 of SEQ ID NO: 18.

In any embodiment, the antibody-based molecule that binds to human CEMIP comprises a heavy chain variable region comprising the CDR-H1 of SEQ ID NO: 5, the CDR-H2 of SEQ ID NO: 12, and the CDR-H3 of SEQ ID NO: 19.

In any embodiment, the antibody-based molecule that binds to human CEMIP comprises a heavy chain variable region comprising the CDR-H1 of SEQ ID NO: 6, the CDR-H2 of SEQ ID NO: 13, and the CDR-H3 of SEQ ID NO: 20.

In any embodiment, the antibody-based molecule that binds to human CEMIP comprises a heavy chain variable region comprising the CDR-H1 of SEQ ID NO: 7, the CDR-H2 of SEQ ID NO: 14, and the CDR-H3 of SEQ ID NO: 21.

In any embodiment, the antibody-based molecule that binds to human CEMIP comprises a heavy chain variable region comprising the CDR-H1 of SEQ ID NO: 8, the CDR-H2 of SEQ ID NO: 15, and the CDR-H3 of SEQ ID NO: 22.

The sequences of the heavy chain CDRs of the CEMIP antibodies disclosed herein are provided in Table 1 below.

TABLE 1
Heavy Chain CDR Sequences of CEMIP Antibodies

| | HCDR1 | | HCDR2 | | HCDR3 | |
|---|---|---|---|---|---|---|
| | | SEQ | | SEQ | | SEQ |
| mAb | | ID | | ID | | ID |
| name | Sequence | NO: | Sequence | NO: | Sequence | NO: |
| cAb4853 | SFGMH | 2 | YISSASNTIYY | 9 | RDWDLYAMDY | 16 |
| 07F11C02 | | | ADTVKG | | | |
| cAb4854 | SFGMH | 3 | YISSASNTIYY | 10 | RNWDLYAMDY | 17 |
| 10F01B02 | | | ADTVKG | | | |
| cAb4855 | TSGMGVG | 4 | HIWWNDEKYYN | 11 | ITGAWFPY | 18 |
| 01F11A01 | | | AALKS | | | |
| cAb5775 | AYTMN | 5 | LINPYNGGTTY | 12 | YDYGYAMDY | 19 |
| 12D02 | | | NQKFKG | | | |
| cAb5776 | AYTMN | 6 | LINPYNGGTTY | 13 | YDYGYAMDY | 20 |
| 12A12 | | | NQKFKG | | | |
| cAb5777 | DTYMH | 7 | NIDPANGHTKY | 14 | SNDYDVDFDY | 21 |
| 12D08 | | | APKFQG | | | |
| cAb5778 | AYTMN | 8 | LINPYNGGTTY | 15 | YYGGRGWYFD | 22 |
| 13G04 | | | NQKFKG | | V | |

In any embodiment, the CEMIP antibody-based molecules as disclosed herein further comprise a light chain variable region. The light chain variable region comprises (i) a complementarity-determining region 1 (CDR-L1) having an amino acid sequence of any one of SEQ ID NOs: 23-29, or a modified amino acid sequence of any one of SEQ ID NO: 23-29, said modified sequence having at least 80%, at least 85%, at least 90%, at least 95%, or at least 98% sequence identity to any one of SEQ ID NO: 23-29; (ii) a complementarity-determining region 2 (CDR-L2) having an amino acid sequence of any one of SEQ ID NOs: 30-36, or a modified amino acid sequence of any one of SEQ ID NO: 30-36, said modified sequence having at least 80%, at least 85%, at least 90%, at least 95%, or at least 98% sequence identity to any one of SEQ ID NO: 30-36; and (iii) a complementarity-determining region 3 (CDR-L3) having an amino acid sequence of any one of SEQ ID NOs: 37-43, or a modified amino acid sequence of any one of SEQ ID NO: 37-43, said modified sequence having at least 80%, at least 85%, at least 90%, at least 95%, or at least 98% sequence identity to any one of SEQ ID NO: 37-43.

In any embodiment, the light chain variable region of the CEMIP antibody-based molecule disclosed herein comprises a light chain variable region comprising the CDR-L1 of SEQ ID NO: 23, the CDR-L2 of SEQ ID NO: 30, and the CDR-L3 of SEQ ID NO: 37.

In any embodiment, the light chain variable region of the CEMIP antibody-based molecule disclosed herein comprises a light chain variable region comprising the CDR-L1 of SEQ ID NO: 24, the CDR-L2 of SEQ ID NO: 31, and the CDR-L3 of SEQ ID NO: 38.

In any embodiment, the light chain variable region of the CEMIP antibody-based molecule disclosed herein comprises a light chain variable region comprising the CDR-L1 of SEQ ID NO: 25, the CDR-L2 of SEQ ID NO: 32, and the CDR-L3 of SEQ ID NO: 39.

In any embodiment, the light chain variable region of the CEMIP antibody-based molecule disclosed herein comprises a light chain variable region comprising the CDR-L1 of SEQ ID NO: 26, the CDR-L2 of SEQ ID NO: 33, and the CDR-L3 of SEQ ID NO: 40.

In any embodiment, the light chain variable region of the CEMIP antibody-based molecule disclosed herein comprises a light chain variable region comprising the CDR-L1 of SEQ ID NO: 27, the CDR-L2 of SEQ ID NO: 34, and the CDR-L3 of SEQ ID NO: 41.

In any embodiment, the light chain variable region of the CEMIP antibody-based molecule disclosed herein comprises a light chain variable region comprising the CDR-L1 of SEQ ID NO: 28, the CDR-L2 of SEQ ID NO: 35, and the CDR-L3 of SEQ ID NO: 42.

In any embodiment, the light chain variable region of the CEMIP antibody-based molecule disclosed herein comprises a light chain variable region comprising the CDR-L1 of SEQ ID NO: 29, the CDR-L2 of SEQ ID NO: 36, and the CDR-L3 of SEQ ID NO: 43.

The sequences of the light chain CDRs of the CEMIP antibodies disclosed herein are provided in Table 2 below.

TABLE 2
Light Chain CDR Sequences of CEMIP Antibodies

| | LCDR1 | | LCDR2 | | LCDR3 | |
|---|---|---|---|---|---|---|
| | | SEQ | | SEQ | | SEQ |
| mAb | | ID | | ID | | ID |
| name | Sequence | NO: | Sequence | NO: | Sequence | NO: |
| cAb4853 | KASQNVGTAVA | 23 | SASNRHT | 30 | QQYSSSPT | 37 |
| 07F11C02 | | | | | | |
| cAb4854 | KANQNVGTAVA | 24 | STSNRDT | 31 | QQYRNYPT | 38 |
| 10F01B02 | | | | | | |
| cAb4855 | RASQHISEYLH | 25 | YGSQSIS | 32 | QNGHSFPYT | 39 |
| 01F11A01 | | | | | | |
| cAb5775 | RSSQSIVHRNG | 26 | KVSNRFS | 33 | FQGSHVPWT | 40 |
| 12D02 | NTYLE | | | | | |
| cAb5776 | RSSQSIVHRSG | 27 | KVSNRFS | 34 | FQGSHVPWT | 41 |
| 12A12 | NTYLE | | | | | |
| cAb5777 | KSSQSLLNSRT | 28 | WASTRES | 35 | KQSYNLYT | 42 |
| 12D08 | RKNYLA | | | | | |
| cAb5778 | RASKSVSTSGY | 29 | LASNLES | 36 | QNSRELPYT | 43 |
| 13G04 | SYVH | | | | | |

Suitable amino acid modifications to the heavy chain CDR sequences and/or the light chain CDR sequences of the CEMIP antibody-based molecule disclosed herein include, for example, conservative substitutions or functionally equivalent amino acid residue substitutions that result in variant CDR sequences having similar or enhanced binding characteristics to those of the CDR sequences disclosed herein as described above. Encompassed by the present disclosure are CDRs of Table 1 and 2 containing 1, 2, 3, 4, 5, or more amino acid substitutions (depending on the length of the CDR) that maintain or enhance CEMIP binding of the antibody. The resulting modified CDRs are at least 70%, at least 75%, at least 80%, at least 85%, at least 90%, at least 95%, at least 98% similar in sequence to the CDRs of Tables 1 and 2. Suitable amino acid modifications to the heavy chain CDR sequences of Table 1 and/or the light chain CDR sequences of Tables 1 and 2 include, for example, conservative substitutions or functionally equivalent amino acid residue substitutions that result in variant CDR sequences having similar or enhanced binding characteristics to those of the CDR sequences of Table 1 and Table 2. Conservative substitutions are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. Alternatively, the amino acid repertoire can be grouped as (1) acidic (aspartate, glutamate); (2) basic (lysine, arginine histidine), (3) aliphatic (glycine, alanine, valine, leucine, isoleucine, serine, threonine), with serine and threonine optionally grouped separately as aliphatic-hydroxyl; (4) aromatic (phenylalanine, tyrosine, tryptophan); (5) amide (asparagine, glutamine); and (6) sulfur-containing (cysteine and methionine) (Stryer (ed.), Biochemistry, 2nd ed, WH Freeman and Co., 1981, which is hereby incorporated by reference in its entirety). Non-conservative substitutions can also be made to the heavy chain CDR sequences of Table 1 and the light chain CDR sequences of Table 2. Non-conservative substitutions involve substituting one or more amino acid residues of the CDR with one or more amino acid residues from a different class of amino acids to improve or enhance the binding properties of CDR. The amino acid sequences of the heavy chain variable region CDRs of Table 1 and/or the light chain variable region CDRs of Table 2 may further comprise one or more internal neutral amino acid insertions or deletions that maintain or enhance CEMIP binding.

In any embodiment, the antibody-based molecule that binds to human CEMIP comprises a heavy chain variable region comprising the CDR-H1 of SEQ ID NO: 2, the CDR-H2 of SEQ ID NO: 9, and the CDR-H3 of SEQ ID NO: 16, and a light chain variable region comprising the CDR-L1 of SEQ ID NO: 23, the CDR-L2 of SEQ ID NO: 30, and the CDR-L3 of SEQ ID NO: 37.

In any embodiment, the antibody-based molecule that binds to human CEMIP comprises a heavy chain variable region comprising the CDR-H1 of SEQ ID NO: 3, the CDR-H2 of SEQ ID NO: 10, and the CDR-H3 of SEQ ID NO: 17, and a light chain variable region comprising the CDR-L1 of SEQ ID NO: 24, the CDR-L2 of SEQ ID NO: 31, and the CDR-L3 of SEQ ID NO: 38.

In any embodiment, the antibody-based molecule that binds to human CEMIP comprises a heavy chain variable region comprising the CDR-H1 of SEQ ID NO: 4, the CDR-H2 of SEQ ID NO: 11, and the CDR-H3 of SEQ ID NO: 18, and a light chain variable region comprising the CDR-L1 of SEQ ID NO: 25, the CDR-L2 of SEQ ID NO: 32, and the CDR-L3 of SEQ ID NO: 39.

In any embodiment, the antibody-based molecule that binds to human CEMIP comprises a heavy chain variable region comprising the CDR-H1 of SEQ ID NO: 5, the CDR-H2 of SEQ ID NO: 12, and the CDR-H3 of SEQ ID NO: 19, and a light chain variable region comprising the CDR-L1 of SEQ ID NO: 26, the CDR-L2 of SEQ ID NO: 33, and the CDR-L3 of SEQ ID NO: 40.

In any embodiment, the antibody-based molecule that binds to human CEMIP comprises a heavy chain variable region comprising the CDR-H1 of SEQ ID NO: 6, the CDR-H2 of SEQ ID NO: 13, and the CDR-H3 of SEQ ID NO: 20, and a light chain variable region comprising the CDR-L1 of SEQ ID NO: 27, the CDR-L2 of SEQ ID NO: 34, and the CDR-L3 of SEQ ID NO: 41.

In any embodiment, the antibody-based molecule that binds to human CEMIP comprises a heavy chain variable region comprising the CDR-H1 of SEQ ID NO: 7, the CDR-H2 of SEQ ID NO: 14, and the CDR-H3 of SEQ ID NO: 21, and a light chain variable region comprising the CDR-L1 of SEQ ID NO: 28, the CDR-L2 of SEQ ID NO: 35, and the CDR-L3 of SEQ ID NO: 42.

In any embodiment, the antibody-based molecule that binds to human CEMIP comprises a heavy chain variable region comprising the CDR-H1 of SEQ ID NO: 8, the CDR-H2 of SEQ ID NO: 15, and the CDR-H3 of SEQ ID NO: 22, and a light chain variable region comprising the CDR-L1 of SEQ ID NO: 29, the CDR-L2 of SEQ ID NO: 36, and the CDR-L3 of SEQ ID NO: 43.

The CEMIP antibody-based molecule as described herein may comprise a variable light (VL) chain, a variable heavy (VH) chain, or a combination of VL and VH chains. In any embodiment, the VH chain of the CEMIP antibody-based molecule comprises any one of the VH amino acid sequences provided in Table 3 below, or an amino acid sequence that is at least 60%, at least 65%, at least 70%, at least 75%, at least 80%, at least 85%, at least 90%, at least 95% identical to any one of the VH amino acid sequences listed in Table 3. In any embodiment, the VL chain of the CEMIP antibody-based molecule comprises any one of the VL amino acid sequences provided in Table 3 below, or an amino acid sequence that is at least 60%, at least 65%, at least 70%, at least 75%, at least 80%, at least 85%, at least 90%, at least 95% identical to any one of the VL amino acid sequences listed in Table 3.

TABLE 3
CEMIP Antibody Variable Heavy (VH) and
Variable Light (VL) Antibody Sequences

| mAb | | | SEQ ID |
|---|---|---|---|
| name | Region | Sequence | NO: |
| cAb4853 | VH | DVQLVESGGGLVQPGGSRKLSCAASGFAFSSFGMHWVRQA | 44 |
| | IgG2a | PERGLEWVAYISSASNTIYYADTVKGRFTISRDNPKSTLF | |
| | | LQMTSLRSEDTAIYYCAKRDWDLYAMDYWGQGTSVTVSS | |
| | VL | DIVMTQSQKFLSTSVGDRVTITCKASQNVGTAVAWYQQKP | 45 |
| | kappa | GQSPELLIYSASNRHTGVPARFTGSGSGTDFTLTITNMQS | |
| | | EDLADYFCQQYSSSPTFGGGTKLESK | |
| cAb4854 | VH | DVQLVESGGGLVQPGGSRKLSCAASGFPFSSFGMHWVRQA | 46 |
| | IgG2a | PDKGLEWVAYISSASNTIYYADTVKGRFTVSRDNPKNTLF | |
| | | LQMTSLRSEDTAIYYCTKRNWDLYAMDYWGQGTSVTVSS | |
| | VL | DIVMTQSQKFMSTSVGDRVSITCKANQNVGTAVAWYQQKP | 47 |
| | kappa | GQSPKLLIYSTSNRDTGVPDRFTGSGSGTDFTLTINYIQS | |
| | | EDLADYFCQQYRNYPTFGGGTKLEIK | |
| cAb4855 | VH | QVTLKESGPGILQPSQTLSLTCSFSGFSLSTSGMGVGWIR | 48 |
| | IgG2a | QPSGKGLEWLAHIWWNDEKYYNAALKSRLTISKDTSKNQV | |
| | | FLKIASVDAADTATYYCARITGAWFPYWGQGTLVTVSA | |
| | VL | DIVMTQSPATLSVTPGDRVSLSCRASQHISEYLHWYQQKS | 49 |
| | kappa | HESPRLLIKYGSQSISGIPSRFSGSGSGSDFTLNINSVEP | |
| | | EDVGVYYCQNGHSFPYTFGGGTKLEIK | |
| cAb5775 | VH | EVQLQQSGPELVKPGASMKISCKASGYSFTAYTMNWVKQS | 50 |
| | IgG1 | HGENLEWIGLINPYNGGTTYNQKFKGKATLTVDKSSSTAY | |
| | | MELLSLTSEDSAVYYCASYDYGYAMDYWGQGTSVTVSS | |
| | VL | DVLMTQTPLSLPVSLGDQASISCRSSQSIVHRNGNTYLEW | 51 |
| | kappa | YLQKPGQSPKLLIYKVSNRFSGVPDRFSGSGSGTDFTLKI | |
| | | SRVEAEDLGVYYCFQGSHVPWTFGGGTKLEIK | |
| cAb5776 | VH | EVQLQQSGPELVKPGVSMKISCKASGYSFTAYTMNWVKQS | 52 |
| | IgG1 | HGENLEWIGLINPYNGGTTYNQKFKGKATLTVDKSSSTAY | |
| | | MELLSLTSEDSAVYYCASYDYGYAMDYWGQGTSVTVSS | |
| | VL | DVLMTQTPLSLPVSLGDQASISCRSSQSIVHRSGNTYLEW | 53 |
| | kappa | YLQKPGQSPKLLIYKVSNRFSGVPDRFSGSGSGTDFTLKI | |
| | | SRVEAEDLGVYYCFQGSHVPWTFGGGTKLEIK | |
| cAb5777 | VH | EVQLQQSGAELVKPGASVKLSCTASGFNIKDTYMHWVKQR | 54 |
| | IgG2a | PEQGLIWIGNIDPANGHTKYAPKFQGKATITADTSSNTAY | |
| | | LQLSSLTSEDTAVYYCARSNDYDVDFDYWGQGTTLTVSS | |
| | VL | DIVMSQSPSSLAVSAGEKVTMSCKSSQSLLNSRTRKNYLA | 55 |
| | kappa | WYQQKPGQSPKLLIYWASTRESGVPDRFTGSGSGTDFSLT | |
| | | ISSVQAEDLAVYYCKQSYNLYTFGGGTKLEIK | |
| cAb5778 | VH | EVHLQQSGPELVKPGASMKISCKASGYSFTAYTMNWVKQS | 56 |
| | IgG1 | HGKNLEWIGLINPYNGGTTYNQKFKGKATLTVDKSSSTAY | |
| | | MELLSLTSEDSAVYYCASYYGGRGWYFDVWGAGTSVTVSS | |
| | VL | DIVLTQSPASLAVSLGQRATISCRASKSVSTSGYSYVHWY | 57 |
| | kappa | QQKPGQPPKLLIYLASNLESGVPARFSGSGSGTDFTLNIH | |
| | | PVEEEDAATYYCONSRELPYTFGGGTKLEMK | |

In any embodiment, the CEMIP antibody-based molecule disclosed herein comprises a heavy chain variable region comprising an amino acid sequence that is at least 80% identical to SEQ ID NO: 44 and a light chain variable region comprising an amino acid sequence that is at least 80% identical to SEQ ID NO: 45.

In any embodiment, the CEMIP antibody-based molecule disclosed herein comprises a heavy chain variable region comprising an amino acid sequence that is at least 80% identical to SEQ ID NO: 46 and a light chain variable region comprising an amino acid sequence that is at least 80% identical to SEQ ID NO: 47.

In any embodiment, the CEMIP antibody-based molecule disclosed herein comprises a heavy chain variable region comprising an amino acid sequence that is at least 80% identical to SEQ ID NO: 48 and a light chain variable region comprising an amino acid sequence that is at least 80% identical to SEQ ID NO: 49.

In any embodiment, the CEMIP antibody-based molecule disclosed herein comprises a heavy chain variable region comprising an amino acid sequence that is at least 80% identical to SEQ ID NO: 50 and a light chain variable region comprising an amino acid sequence that is at least 80% identical to SEQ ID NO: 51.

In any embodiment, the CEMIP antibody-based molecule disclosed herein comprises (v) a heavy chain variable region comprising an amino acid sequence that is at least 80% identical to SEQ ID NO: 52 and a light chain variable region comprising an amino acid sequence that is at least 80% identical to SEQ ID NO: 53.

In any embodiment, the CEMIP antibody-based molecule disclosed herein comprises a heavy chain variable region comprising an amino acid sequence that is at least 80% identical to SEQ ID NO: 54 and a light chain variable region comprising an amino acid sequence that is at least 80% identical to SEQ ID NO: 55.

In any embodiment, the CEMIP antibody-based molecule disclosed herein comprises a heavy chain variable region comprising an amino acid sequence that is at least 80% identical to SEQ ID NO: 56 and a light chain variable region comprising an amino acid sequence that is at least 80% identical to SEQ ID NO: 57.

In any embodiment, the CEMIP antibody-based molecule disclosed herein comprises a heavy chain variable region comprising an amino acid sequence that is at least 80% identical to SEQ ID NO: 44, a light chain variable region comprising an amino acid sequence that is at least 80% identical to SEQ ID NO: 45, a heavy chain constant region comprising an amino acid sequence that is at least 80% identical to SEQ ID NO: 72, and a light chain constant region comprising an amino acid sequence that is at least 80% identical to SEQ ID NO: 73. This antibody is referred to herein as cAb4853.

In any embodiment, the CEMIP antibody-based molecule disclosed herein comprises a heavy chain variable region comprising an amino acid sequence that is at least 80% identical to SEQ ID NO: 46, a light chain variable region comprising an amino acid sequence that is at least 80% identical to SEQ ID NO: 47, a heavy chain constant region comprising an amino acid sequence that is at least 80% identical to SEQ ID NO: 74, and a light chain constant region comprising an amino acid sequence that is at least 80% identical to SEQ ID NO: 75. This antibody is referred to herein as cAb4854.

In any embodiment, the CEMIP antibody-based molecule disclosed herein comprises a heavy chain variable region comprising an amino acid sequence that is at least 80% identical to SEQ ID NO: 48, a light chain variable region comprising an amino acid sequence that is at least 80% identical to SEQ ID NO: 49, a heavy chain constant region comprising an amino acid sequence that is at least 80% identical to SEQ ID NO: 76, and a light chain constant region comprising an amino acid sequence that is at least 80% identical to SEQ ID NO: 77. This antibody is referred to herein as cAb4855.

In any embodiment, the CEMIP antibody-based molecule disclosed herein comprises a heavy chain variable region comprising an amino acid sequence that is at least 80% identical to SEQ ID NO: 50, a light chain variable region comprising an amino acid sequence that is at least 80% identical to SEQ ID NO: 51, a heavy chain constant region comprising an amino acid sequence that is at least 80% identical to SEQ ID NO: 78, and a light chain constant region comprising an amino acid sequence that is at least 80% identical to SEQ ID NO: 79. This antibody is referred to herein as cAb5775.

In any embodiment, the CEMIP antibody-based molecule disclosed herein comprises a heavy chain variable region comprising an amino acid sequence that is at least 80% identical to SEQ ID NO: 52, a light chain variable region comprising an amino acid sequence that is at least 80% identical to SEQ ID NO: 53, a heavy chain constant region comprising an amino acid sequence that is at least 80% identical to SEQ ID NO: 80, and a light chain constant region comprising an amino acid sequence that is at least 80% identical to SEQ ID NO: 81. This antibody is referred to herein as cAb5776.

In any embodiment, the CEMIP antibody-based molecule disclosed herein comprises a heavy chain variable region comprising an amino acid sequence that is at least 80% identical to SEQ ID NO: 54, a light chain variable region comprising an amino acid sequence that is at least 80% identical to SEQ ID NO: 55, a heavy chain constant region comprising an amino acid sequence that is at least 80% identical to SEQ ID NO: 82, and a light chain constant region comprising an amino acid sequence that is at least 80% identical to SEQ ID NO: 83. This antibody is referred to herein as cAb5777.

In any embodiment, the CEMIP antibody-based molecule disclosed herein comprises a heavy chain variable region comprising an amino acid sequence that is at least 80% identical to SEQ ID NO: 56 and a light chain variable region comprising an amino acid sequence that is at least 80% identical to SEQ ID NO: 57, a heavy chain constant region comprising an amino acid sequence that is at least 80% identical to SEQ ID NO: 84, and a light chain constant region comprising an amino acid sequence that is at least 80% identical to SEQ ID NO: 85. This antibody is referred to herein as cAb5778.

Another aspect of the present disclosure is directed to isolated polynucleotides encoding the CEMIP antibody-based molecules described herein. In one embodiment, the polynucleotide encoding the CEMIP antibody of the present disclosure comprises a sequence encoding any one, any two, any three, any four, any five, or any six of the CDRs described supra, including the heavy chain CDRs of SEQ ID NOs: 2-22, and the light chain CDRs of SEQ ID NOs: 23-43.

In any embodiment, the polynucleotide comprises a nucleotide sequence encoding a V_{H} domain, where the V_{H} domain comprises the CDR-H1 of SEQ ID NO: 2, the CDR-H2 of SEQ ID NO: 9, and the CDR-H3 of SEQ ID NO: 16. An exemplary nucleotide sequence comprises the nucleotide sequence of SEQ ID NO: 58, and nucleotide sequences having at least 80%, at least 85%, at least 90%, at least 95%, at least 96%, 97%, 98%, or 99% sequence similarity to the nucleotide sequence of SEQ ID NO: 58.

In any embodiment, the polynucleotide comprises a nucleotide sequence encoding a V_{H} domain, where the V_{H} domain comprises the CDR-H1 of SEQ ID NO: 3, the CDR-H2 of SEQ ID NO: 10, and the CDR-H3 of SEQ ID NO: 17. An exemplary nucleotide sequence comprises the nucleotide sequence of SEQ ID NO: 60, and nucleotide sequences having at least 80%, at least 85%, at least 90%, at least 95%, at least 96%, 97%, 98%, or 99% sequence similarity to the nucleotide sequence of SEQ ID NO: 60.

In any embodiment, the polynucleotide comprises a nucleotide sequence encoding a V_{H} domain, where the V_{H} domain comprises the CDR-H1 of SEQ ID NO: 4, the CDR-H2 of SEQ ID NO: 11, and the CDR-H3 of SEQ ID NO: 18. An exemplary nucleotide sequence comprises the nucleotide sequence of SEQ ID NO: 62, and nucleotide sequences having at least 80%, at least 85%, at least 90%, at least 95%, at least 96%, 97%, 98%, or 99% sequence similarity to the nucleotide sequence of SEQ ID NO: 62.

In any embodiment, the polynucleotide comprises a nucleotide sequence encoding a V_{H} domain, where the V_{H} domain comprises the CDR-H1 of SEQ ID NO: 5, the CDR-H2 of SEQ ID NO: 12, and the CDR-H3 of SEQ ID NO: 19. An exemplary nucleotide sequence comprises the nucleotide sequence of SEQ ID NO: 64, and nucleotide sequences having at least 80%, at least 85%, at least 90%, at least 95%, at least 96%, 97%, 98%, or 99% sequence similarity to the nucleotide sequence of SEQ ID NO: 64.

In any embodiment, the polynucleotide comprises a nucleotide sequence encoding a V_{H} domain, where the V_{H} domain comprises the CDR-H1 of SEQ ID NO: 6, the CDR-H2 of SEQ ID NO: 13, and the CDR-H3 of SEQ ID NO: 20. An exemplary nucleotide sequence comprises the nucleotide sequence of SEQ ID NO: 66, and nucleotide sequences having at least 80%, at least 85%, at least 90%, at least 95%, at least 96%, 97%, 98%, or 99% sequence similarity to the nucleotide sequence of SEQ ID NO: 66.

In any embodiment, the polynucleotide comprises a nucleotide sequence encoding a V_{H} domain, where the V_{H} domain comprises the CDR-H1 of SEQ ID NO: 7, the CDR-H2 of SEQ ID NO: 14, and the CDR-H3 of SEQ ID NO: 21. An exemplary nucleotide sequence comprises the nucleotide sequence of SEQ ID NO: 68, and nucleotide sequences having at least 80%, at least 85%, at least 90%, at least 95%, at least 96%, 97%, 98%, or 99% sequence similarity to the nucleotide sequence of SEQ ID NO: 68.

In any embodiment, the polynucleotide comprises a nucleotide sequence encoding a V_{H} domain, where the V_{H} domain comprises the CDR-H1 of SEQ ID NO: 8, the CDR-H2 of SEQ ID NO: 15, and the CDR-H3 of SEQ ID NO: 22. An exemplary nucleotide sequence comprises the nucleotide sequence of SEQ ID NO: 70, and nucleotide sequences having at least 80%, at least 85%, at least 90%, at least 95%, at least 96%, 97%, 98%, or 99% sequence similarity to the nucleotide sequence of SEQ ID NO: 70.

Exemplary nucleotide sequences of CEMIP V_{H} domains described herein are provided in Table 4 below.

In any embodiment, the polynucleotide of the present disclosure comprises a nucleotide sequence encoding a V_{L} domain. In any embodiment, the polynucleotide comprises a nucleotide sequence encoding a V_{L} domain, where the V_{L} domain comprises the CDR-L1 of SEQ ID NO: 23, the CDR-L2 of SEQ ID NO: 30, and the CDR-L3 of SEQ ID NO: 37. An exemplary nucleotide sequence comprises the nucleotide sequence of SEQ ID NO: 59, and nucleotide sequences having at least 80%, at least 85%, at least 90%, at least 95%, at least 96%, 97%, 98%, or 99% sequence similarity to the nucleotide sequence of SEQ ID NO: 59.

In any embodiment, the polynucleotide comprises a nucleotide sequence encoding a V_{L} domain, where the V_{L} domain comprises the CDR-L1 of SEQ ID NO: 24, the CDR-L2 of SEQ ID NO: 31, and the CDR-L3 of SEQ ID NO: 38. An exemplary nucleotide sequence comprises the nucleotide sequence of SEQ ID NO: 61, and nucleotide sequences having at least 80%, at least 85%, at least 90%, at least 95%, at least 96%, 97%, 98%, or 99% sequence similarity to the nucleotide sequence of SEQ ID NO: 61.

In any embodiment, the polynucleotide comprises a nucleotide sequence encoding a V_{L} domain, where the V_{L} domain comprises the CDR-L1 of SEQ ID NO: 25, the CDR-L2 of SEQ ID NO: 32, and the CDR-L3 of SEQ ID NO: 39. An exemplary nucleotide sequence comprises the nucleotide sequence of SEQ ID NO: 63, and nucleotide sequences having at least 80%, at least 85%, at least 90%, at least 95%, at least 96%, 97%, 98%, or 99% sequence similarity to the nucleotide sequence of SEQ ID NO: 63.

In any embodiment, the polynucleotide comprises a nucleotide sequence encoding a V_{L} domain, where the V_{L} domain comprises the CDR-L1 of SEQ ID NO: 26, the CDR-L2 of SEQ ID NO: 33, and the CDR-L3 of SEQ ID NO: 40. An exemplary nucleotide sequence comprises the nucleotide sequence of SEQ ID NO: 65, and nucleotide sequences having at least 80%, at least 85%, at least 90%, at least 95%, at least 96%, 97%, 98%, or 99% sequence similarity to the nucleotide sequence of SEQ ID NO: 65.

In any embodiment, the polynucleotide comprises a nucleotide sequence encoding a V_{L} domain, where the V_{L} domain comprises the CDR-L1 of SEQ ID NO: 27, the CDR-L2 of SEQ ID NO: 34, and the CDR-L3 of SEQ ID NO: 41. An exemplary nucleotide sequence comprises the nucleotide sequence of SEQ ID NO: 67, and nucleotide sequences having at least 80%, at least 85%, at least 90%, at least 95%, at least 96%, 97%, 98%, or 99% sequence similarity to the nucleotide sequence of SEQ ID NO: 67.

In any embodiment, the polynucleotide comprises a nucleotide sequence encoding a V_{L} domain, where the V_{L} domain comprises the CDR-L1 of SEQ ID NO: 28, the CDR-L2 of SEQ ID NO: 35, and the CDR-L3 of SEQ ID NO: 42. An exemplary nucleotide sequence comprises the nucleotide sequence of SEQ ID NO: 69, and nucleotide sequences having at least 80%, at least 85%, at least 90%, at least 95%, at least 96%, 97%, 98%, or 99% sequence similarity to the nucleotide sequence of SEQ ID NO: 69.

In s any embodiment, the polynucleotide comprises a nucleotide sequence encoding a V_{L} domain, where the V_{L} domain comprises the CDR-L1 of SEQ ID NO: 29, the CDR-L2 of SEQ ID NO: 36, and the CDR-L3 of SEQ ID NO: 43. An exemplary nucleotide sequence comprises the nucleotide sequence of SEQ ID NO: 71, and nucleotide sequences having at least 80%, at least 85%, at least 90%, at least 95%, at least 96%, 97%, 98%, or 99% sequence similarity to the nucleotide sequence of SEQ ID NO: 71.

Exemplary nucleotide sequences of CEMIP V_{L} domains described herein are provided in Table 4 below.

TABLE 4
CEMIP Antibody Variable Heavy (VH) and Variable
Light (VL) DNA Sequences

| mAb/Fab | | | SEQ ID |
|---|---|---|---|
| name | Region | Sequence | NO: |
| cAb4853 | VH | GATGTGCAGCTGGTGGAGTCTGGGGGAGGCTTAGTGCAGCCTGGAGGGT | 58 |
| | | CCCGGAAACTCTCCTGTGCAGCCTCTGGATTCGCTTTCAGTAGCTTTGG | |
| | | AATGCACTGGGTTCGTCAGGCTCCAGAGAGGGGGCTGGAGTGGGTCGCA | |
| | | TACATTAGTAGTGCCAGCAATACCATCTACTATGCAGACACAGTGAAGG | |
| | | GCCGATTCACCATCTCCAGAGACAATCCCAAGAGCACCCTGTTCCTGCA | |
| | | AATGACCAGTCTAAGGTCTGAGGACACGGCCATTTATTACTGTGCAAAG | |
| | | CGAGACTGGGACCTCTATGCTATGGACTACTGGGGTCAAGGAACCTCAG | |
| | | TCACCGTCTCGTCA | |
| | VL | GACATTGTGATGACCCAGTCTCAAAAATTCTTGTCCACATCAGTGGGAG | 59 |
| | | ACAGGGTCACCATCACCTGCAAGGCCAGTCAGAATGTGGGAACTGCTGT | |
| | | TGCCTGGTATCAACAGAAACCAGGGCAATCTCCTGAACTTCTGATTTAC | |
| | | TCGGCATCCAATCGGCACACTGGAGTCCCTGCTCGCTTCACAGGCAGTG | |
| | | GATCTGGGACAGATTTCACTCTCACCATCACCAATATGCAGTCTGAAGA | |
| | | CCTGGCAGATTATTTCTGCCAGCAATATAGTAGCTCGCCGACATTCGGT | |
| | | GGAGGCACCAAGTTGGAATCCAAA | |
| cAb4854 | VH | GATGTGCAGCTGGTGGAGTCTGGGGGAGGCTTAGTGCAGCCTGGAGGGT | 60 |
| | | CCCGGAAACTCTCCTGTGCAGCCTCTGGATTCCCTTTCAGTAGCTTTGG | |
| | | AATGCACTGGGTTCGTCAGGCTCCAGACAAGGGACTGGAGTGGGTCGCA | |
| | | TACATTAGTAGTGCCAGTAATACCATCTACTATGCTGACACAGTGAAGG | |
| | | GCCGATTCACCGTCTCCAGAGACAATCCCAAGAACACCCTGTTCCTGCA | |
| | | AATGACCAGTCTAAGGTCTGAGGACACGGCCATTTATTACTGTACAAAG | |
| | | CGAAATTGGGACCTCTATGCTATGGACTACTGGGGTCAAGGAACCTCAG | |
| | | TCACCGTCTCCTCA | |
| | VL | GACATTGTGATGACCCAGTCTCAAAAATTCATGTCCACATCAGTGGGAG | 61 |
| | | ACAGGGTCAGCATCACCTGCAAGGCCAATCAGAATGTGGGTACTGCTGT | |
| | | AGCCTGGTATCAACAGAAACCAGGACAATCTCCTAAACTACTGATTTAT | |
| | | TCGACATCCAATCGGGACACTGGAGTCCCTGACCGCTTCACAGGCAGTG | |
| | | GATCTGGGACAGATTTCACTCTCACCATCAACTATATTCAATCTGAAGA | |
| | | CCTGGCAGATTATTTCTGCCAGCAATATAGAAATTATCCGACGTTCGGT | |
| | | GGAGGCACCAAGCTGGAAATCAAA | |
| cAb4855 | VH | CAGGTTACTCTGAAAGAGTCTGGCCCTGGGATATTGCAGCCCTCCCAGA | 62 |
| | | CCCTCAGTCTGACTTGTTCTTTCTCTGGGTTTTCACTGAGCACTTCTGG | |
| | | TATGGGTGTAGGCTGGATTCGTCAGCCTTCAGGGAAGGGTCTGGAGTGG | |
| | | CTGGCACACATTTGGTGGAATGATGAGAAATACTATAACGCAGCCCTGA | |
| | | AGAGCCGGCTCACAATCTCCAAGGATACCTCCAAAAACCAGGTTTTCCT | |
| | | CAAGATCGCCAGTGTGGACGCTGCAGATACTGCCACATACTACTGTGCT | |
| | | CGCATCACTGGTGCCTGGTTTCCTTATTGGGGCCAAGGGACTCTGGTCA | |
| | | CTGTCTCTGCA | |
| | VL | GACATTGTGATGACTCAGTCTCCAGCCACCCTGTCTGTGACTCCAGGAG | 63 |
| | | ATAGAGTCTCTCTTTCCTGCAGGGCCAGTCAGCATATTAGCGAATACTT | |
| | | ACACTGGTATCAACAAAAATCACACGAGTCTCCAAGGCTTCTCATCAAA | |
| | | TATGGTTCCCAATCCATCTCTGGGATCCCCTCCAGGTTCAGTGGCAGTG | |
| | | GATCAGGGTCAGATTTCACTCTCAATATCAACAGTGTGGAACCTGAAGA | |
| | | TGTTGGAGTGTATTACTGTCAAAATGGTCACAGCTTTCCGTACACGTTC | |
| | | GGAGGGGGGACCAAGCTGGAAATAAAA | |
| cAb5775 | VH | GAGGTCCAGCTGCAACAGTCTGGACCTGAGCTGGTGAAGCCTGGAGCCT | 64 |
| | | CAATGAAGATATCTTGCAAGGCTTCTGGTTACTCATTCACTGCCTACAC | |
| | | CATGAACTGGGTGAAGCAGAGCCATGGAGAGAACCTTGAGTGGATTGGA | |
| | | CTTATTAATCCTTATAATGGTGGTACTACCTACAACCAGAAGTTCAAGG | |
| | | GCAAGGCCACATTAACTGTAGACAAGTCATCCAGCACAGCCTACATGGA | |
| | | GCTCCTCAGTCTGACATCTGAGGACTCTGCAGTCTATTACTGTGCATCA | |
| | | TATGATTACGGCTATGCTATGGACTACTGGGGTCAAGGAACCTCAGTCA | |
| | | CCGTCTCCTCA | |
| | VL | GATGTTTTGATGACCCAAACTCCACTCTCCCTGCCTGTCAGTCTTGGAG | 65 |
| | | ATCAAGCCTCCATCTCTTGCAGATCTAGTCAGAGCATTGTACATAGAAA | |
| | | TGGAAACACCTATCTAGAATGGTACCTGCAGAAACCAGGCCAGTCTCCA | |
| | | AAGCTCCTGATCTACAAAGTTTCCAACCGATTTTCTGGGGTCCCAGACA | |
| | | GGTTCAGTGGCAGTGGATCAGGGACAGATTTCACACTCAAGATCAGCAG | |
| | | AGTGGAGGCTGAGGATCTGGGAGTTTATTACTGCTTTCAAGGTTCACAT | |
| | | GTTCCGTGGACGTTCGGTGGAGGCACCAAGCTGGAAATCAAA | |
| cAb5776 | VH | GAGGTCCAGCTGCAACAGTCTGGACCTGAGCTGGTGAAGCCTGGAGTTT | 66 |
| | | CAATGAAGATATCCTGCAAGGCTTCTGGTTACTCATTCACTGCCTACAC | |
| | | TATGAATTGGGTGAAGCAGAGTCATGGAGAGAACCTTGAGTGGATTGGA | |
| | | CTTATTAATCCTTATAATGGTGGTACTACCTACAACCAGAAGTTCAAGG | |
| | | GCAAGGCCACATTAACTGTAGACAAGTCATCCAGCACAGCCTACATGGA | |
| | | GCTCCTCAGTCTGACATCTGAGGACTCTGCAGTCTATTACTGTGCATCA | |
| | | TATGATTACGGCTATGCTATGGACTACTGGGGTCAAGGAACCTCAGTCA | |
| | | CCGTCTCCTCA | |
| | VL | GATGTTTTGATGACCCAAACTCCACTCTCCCTGCCTGTCAGTCTTGGAG | 67 |
| | | ATCAAGCCTCCATCTCTTGCAGATCTAGTCAGAGCATTGTACATCGTAG | |
| | | TGGAAACACCTATTTAGAATGGTACCTGCAGAAACCAGGCCAGTCTCCA | |
| | | AAGCTCCTGATCTACAAAGTTTCCAACCGATTTTCTGGGGTCCCAGACA | |
| | | GGTTCAGTGGCAGTGGATCAGGGACAGATTTCACACTCAAGATCAGCAG | |
| | | AGTGGAGGCTGAGGATCTGGGAGTTTATTACTGCTTTCAAGGTTCACAT | |
| | | GTTCCGTGGACGTTCGGTGGAGGCACCAAGTTGGAAATCAAA | |
| cAb5777 | VH | GAGGTTCAACTGCAGCAGTCTGGGGCAGAGCTTGTGAAGCCAGGGGCCT | 68 |
| | | CAGTCAAGTTGTCCTGCACAGCTTCTGGCTTCAACATTAAAGACACCTA | |
| | | TATGCACTGGGTGAAACAGAGGCCTGAGCAGGGCCTGATTTGGATTGGA | |
| | | AACATTGATCCTGCGAATGGTCATACTAAATATGCCCCGAAGTTCCAGG | |
| | | GCAAGGCCACTATCACAGCAGACACATCCTCCAACACAGCCTACCTGCA | |
| | | GCTCAGCAGCCTGACATCTGAGGACACTGCCGTCTATTACTGTGCTAGA | |
| | | TCTAATGATTACGACGTCGACTTTGACTACTGGGGCCAAGGCACCACTC | |
| | | TCACAGTCTCCTCA | |
| | VL | GACATTGTGATGTCACAGTCTCCATCCTCCCTGGCTGTGTCAGCAGGAG | 69 |
| | | AGAAGGTCACTATGAGCTGCAAATCCAGTCAGAGTCTGCTCAACAGTAG | |
| | | AACCCGAAAGAACTACTTGGCTTGGTACCAGCAGAAACCAGGGCAGTCT | |
| | | CCTAAACTGCTGATCTACTGGGCATCCACTAGGGAATCTGGGGTCCCTG | |
| | | ATCGCTTCACAGGCAGTGGATCTGGGACAGATTTCAGTCTCACCATCAG | |
| | | CAGTGTGCAGGCTGAAGACCTGGCAGTTTATTACTGCAAGCAATCTTAT | |
| | | AATCTATACACGTTCGGAGGGGGGACCAAGCTGGAAATAAAA | |
| cAb5778 | VH | GAGGTCCACCTGCAACAGTCTGGACCTGAGCTGGTGAAGCCTGGAGCTT | 70 |
| | | CAATGAAGATATCCTGCAAGGCTTCTGGTTACTCCTTCACTGCCTACAC | |
| | | CATGAACTGGGTGAAACAGAGCCATGGAAAGAACCTTGAGTGGATTGGA | |
| | | CTTATTAATCCTTACAATGGTGGTACTACCTACAACCAGAAATTCAAGG | |
| | | GCAAGGCCACATTAACTGTAGACAAGTCATCCAGCACAGCCTACATGGA | |
| | | GCTCCTCAGTCTGACATCTGAGGACTCTGCAGTCTATTACTGTGCTTCT | |
| | | TACTACGGTGGTAGGGGCTGGTACTTCGATGTCTGGGGCGCAGGGACCT | |
| | | CGGTCACCGTCTCCTCA | |
| | VL | GACATTGTGCTGACACAGTCTCCTGCTTCCTTAGCTGTATCTCTGGGGC | 71 |
| | | AGAGGGCCACCATCTCATGCAGGGCCAGCAAAAGTGTCAGTACATCTGG | |
| | | CTATAGTTATGTTCACTGGTACCAACAGAAACCAGGACAGCCACCCAAA | |
| | | CTCCTCATCTATCTTGCATCCAACCTAGAATCTGGGGTCCCTGCCAGGT | |
| | | TCAGTGGCAGTGGGTCTGGGACAGACTTCACCCTCAACATCCATCCTGT | |
| | | GGAGGAGGAAGATGCTGCAACCTATTACTGTCAGAATAGTAGGGAACTT | |
| | | CCGTACACGTTCGGAGGGGGGACCAAGCTGGAAATGAAA | |

In one embodiment, the isolated polynucleotide encoding the CEMIP antibody-based molecule encodes any one of the V_{H} and/or V_{L} domain sequences as provided in Table 3 infra.

In any embodiment, the polynucleotide encoding the CEMIP antibody of the present disclosure encodes a heavy chain variable region comprising an amino acid sequence that is at least 80% identical to SEQ ID NO: 44 and a light chain variable region comprising an amino acid sequence that is at least 80% identical to SEQ ID NO: 45. An exemplary polynucleotide of this embodiment comprises the nucleotide sequences of SEQ ID NOs: 58 and 59. In any embodiment, the exemplary polynucleotide encoding the CEMIP antibody further includes one or more of a nucleotide sequence encoding a heavy chain constant region (CH), nucleotide sequence encoding a heavy chain signal peptide, a nucleotide sequence encoding the light chain constant region (CL), and a nucleotide sequence encoding light chain signal peptide. Exemplary nucleotide sequences are provided in Table 6 below (see e.g., nucleotides sequences for cAb4853).

In any embodiment, the polynucleotide encoding the CEMIP antibody of the present disclosure encodes a heavy chain variable region comprising an amino acid sequence that is at least 80% identical to SEQ ID NO: 46 and a light chain variable region comprising an amino acid sequence that is at least 80% identical to SEQ ID NO: 47. An exemplary polynucleotide of this embodiment comprises the nucleotide sequences of SEQ ID NOs: 60 and 61. In any embodiment, the exemplary polynucleotide encoding the CEMIP antibody further includes one or more of a nucleotide sequence encoding a heavy chain constant region (CH), a nucleotide sequence encoding a heavy chain signal peptide, a nucleotide sequence encoding the light chain constant region (CL), and a nucleotide sequence encoding light chain signal peptide. Exemplary nucleotide sequences are provided in Table 6 below (see nucleotides sequences for cAb4854).

In any embodiment, the polynucleotide encoding the CEMIP antibody of the present disclosure encodes a heavy chain variable region comprising an amino acid sequence that is at least 80% identical to SEQ ID NO: 48 and a light chain variable region comprising an amino acid sequence that is at least 80% identical to SEQ ID NO: 49. An exemplary polynucleotide of this embodiment comprises the nucleotide sequences of SEQ ID NOs: 62 and 63. In any embodiment, the exemplary polynucleotide encoding the CEMIP antibody further includes one or more of a nucleotide sequence encoding a heavy chain constant region (CH), a nucleotide sequence encoding a heavy chain signal peptide, a nucleotide sequence encoding the light chain constant region (CL), and a nucleotide sequence encoding light chain signal peptide. Exemplary nucleotide sequences are provided in Table 6 below (see nucleotides sequences for cAb4855).

In any embodiment, the polynucleotide encoding the CEMIP antibody of the present disclosure encodes a heavy chain variable region comprising an amino acid sequence that is at least 80% identical to SEQ ID NO: 50 and a light chain variable region comprising an amino acid sequence that is at least 80% identical to SEQ ID NO: 51. An exemplary polynucleotide of this embodiment comprises the nucleotide sequences of SEQ ID NOs: 64 and 65. In any embodiment, the exemplary polynucleotide encoding the CEMIP antibody further includes one or more of a nucleotide sequence encoding a heavy chain constant region (CH), a nucleotide sequence encoding a heavy chain signal peptide, a nucleotide sequence encoding the light chain constant region (CL), and a nucleotide sequence encoding light chain signal peptide. Exemplary nucleotide sequences are provided in Table 6 below (see nucleotides sequences for cAb5775).

In any embodiment, the polynucleotide encoding the CEMIP antibody of the present disclosure encodes a heavy chain variable region comprising an amino acid sequence that is at least 80% identical to SEQ ID NO: 52 and a light chain variable region comprising an amino acid sequence that is at least 80% identical to SEQ ID NO: 53. An exemplary polynucleotide of this embodiment comprises the nucleotide sequences of SEQ ID NOs: 66 and 67. In any embodiment, the exemplary polynucleotide encoding the CEMIP antibody further includes one or more of a nucleotide sequence encoding a heavy chain constant region (CH), a nucleotide sequence encoding a heavy chain signal peptide, a nucleotide sequence encoding the light chain constant region (CL), and a nucleotide sequence encoding light chain signal peptide. Exemplary nucleotide sequences are provided in Table 6 below (see nucleotides sequences for cAb5776).

In any embodiment, the polynucleotide encoding the CEMIP antibody of the present disclosure encodes a heavy chain variable region comprising an amino acid sequence that is at least 80% identical to SEQ ID NO: 54 and a light chain variable region comprising an amino acid sequence that is at least 80% identical to SEQ ID NO: 55. An exemplary polynucleotide of this embodiment comprises the nucleotide sequences of SEQ ID NOs: 68 and 69. In any embodiment, the exemplary polynucleotide encoding the CEMIP antibody further includes one or more of a nucleotide sequence encoding a heavy chain constant region (CH), a nucleotide sequence encoding a heavy chain signal peptide, a nucleotide sequence encoding the light chain constant region (CL), and a nucleotide sequence encoding light chain signal peptide. Exemplary nucleotide sequences are provided in Table 6 below (see nucleotides sequences for cAb5777).

In any embodiment, the polynucleotide encoding the CEMIP antibody of the present disclosure encodes a heavy chain variable region comprising an amino acid sequence that is at least 80% identical to SEQ ID NO: 56 and a light chain variable region comprising an amino acid sequence that is at least 80% identical to SEQ ID NO: 57. An exemplary polynucleotide of this embodiment comprises the nucleotide sequences of SEQ ID NOs: 70 and 71. In any embodiment, the exemplary polynucleotide encoding the CEMIP antibody further includes one or more of a nucleotide sequence encoding a heavy chain constant region (CH), and nucleotide sequence encoding a heavy chain signal peptide, a nucleotide sequence encoding the light chain constant region (CL), and a nucleotide sequence encoding light chain signal peptide. Exemplary nucleotide sequences are provided in Table 6 below (see nucleotides sequences for cAb5778).

The CEMIP nucleic acid molecules described herein include isolated polynucleotides, portions of expression vectors or portions of linear DNA sequences, including linear DNA sequences used for in vitro transcription/translation, and vectors compatible with prokaryotic, eukaryotic or filamentous phage expression, secretion, and/or display of the antibodies or binding fragments thereof described herein.

The CEMIP polynucleotides as described herein may be produced by chemical synthesis such as solid phase polynucleotide synthesis on an automated polynucleotide synthesizer and assembled into complete single or double stranded molecules. Alternatively, the polynucleotides of the disclosure are produced by other techniques such PCR followed by routine cloning. Techniques for producing or obtaining polynucleotides of a given sequence are well known in the art.

The polynucleotides described herein may comprise and/or be operatively coupled to at least one non-coding sequence, such as a promoter or enhancer sequence, intron, polyadenylation signal, a cis sequence facilitating RepA binding, and the like. The polynucleotide sequences may also comprise additional sequences encoding for example a linker sequence, a marker or a tag sequence, such as a histidine tag or an HA tag to facilitate purification or detection of the protein, a signal sequence, a fusion protein partner such as RepA, Fc portion, or bacteriophage coat protein such as pIX or pIII.

Another embodiment of the disclosure is directed to a vector comprising at least one polynucleotide encoding a CEMIP antibody-based molecule as described herein. Such vectors include, without limitation, plasmid vectors, viral vectors, including without limitation, vaccina vector, lentiviral vector, adenoviral vector, adeno-associated viral vector, vectors for baculovirus expression, transposon based vectors or any other vector suitable for introduction of the polynucleotides described herein into a given organism or genetic background by any means to facilitate expression of the encoded CEMIP antibody-based molecule. In one embodiment, the polynucleotide comprises a sequence encoding the heavy chain variable domain, alone or together with a polynucleotide sequence encoding the light chain variable domain as described herein. In any embodiment, the polynucleotides encoding the heavy chain and/or light chain variable domains are operatively coupled with sequences of a promoter, a translation initiation segment (e.g., a ribosomal binding sequence and start codon), a 3′ untranslated region, polyadenylation signal, a termination codon, and transcription termination to form one or more expression vector constructs.

In one embodiment, the vector is an adenoviral-associated viral (AAV) vector. A number of therapeutic AAV vectors suitable for delivery of the polynucleotides encoding antibodies described herein to the central nervous system are known in the art. See e.g., Deverman et al., “Gene Therapy for Neurological Disorders: Progress and Prospects,” Nature Rev. 17:641-659 (2018), which in hereby incorporated by reference in its entirety. Suitable AAV vectors include serotypes AAV1, AAV2, AAV3, AAV4, AAV5, AAV6, AAV7, AAV8, AAV9, AAV10, or AAV11 in their native form or engineered for enhanced tropism. AAV vectors known to have tropism for the CNS that are particularly suited for therapeutic expression of the CEMIP antibodies described herein include, AAV1, AAV2, AAV4, AAV5, AAV8 and AAV9 in their native form or engineered for enhanced tropism. In one embodiment, the AAV vector is an AAV2 vector. In another embodiment, the AAV vector is an AAV5 vector (Vitale et al., “Anti-tau Conformational scFv MCI Antibody Efficiently Reduces Pathological Tau Species in Adult JNPL3 Mice,” Acta Neuropathol. Commun. 6:82 (2018), which is hereby incorporate by reference in its entirety), optionally containing the GFAP or CAG promoter and the Woodchuck hepatitis virus (WPRE) post-translational regulatory element. In another embodiment, the AAV vector is an AAV9 vector (Haiyan et al., “Targeting Root Cause by Systemic scAAV9-hIDS Gene Delivery: Functional Correction and Reversal of Severe MPSII in Mice,” Mol. Ther. Methods Clin. Dev. 10:327-340 (2018), which is hereby incorporated by reference in its entirety). In another embodiment, the AAV vector is an AAVrh10 vector (Liu et al., “Vectored Intracerebral Immunizations with the Anti-Tau Monoclonal Antibody PHF1 Markedly Reduces Tau Pathology in Mutant Transgenic Mice,” J. Neurosci. 36(49): 12425-35 (2016), which is hereby incorporated by reference in its entirety).

In another embodiment the AAV vector is a hybrid vector comprising the genome of one serotype, e.g., AAV2, and the capsid protein of another serotype, e.g., AAV1 or AAV3-9 to control tropism. See e.g., Broekman et al., “Adeno-associated Virus Vectors Serotyped with AAV8 Capsid are More Efficient than AAV-1 or -2 Serotypes for Widespread Gene Delivery to the Neonatal Mouse Brain,” Neuroscience 138:501-510 (2006), which is hereby incorporated by reference in its entirety. In one embodiment, the AAV vector is an AAV2/8 hybrid vector (Ising et al., “AAV-mediated Expression of Anti-Tau ScFv Decreases Tau Accumulation in a Mouse Model of Tauopathy,” J. Exp. Med. 214(5):1227 (2017), which is hereby incorporated by reference in its entirety). In another embodiment the AAV vector is an AAV2/9 hybrid vector (Simon et al., “A Rapid Gene Delivery-Based Mouse Model for Early-Stage Alzheimer Disease-Type Tauopathy,” J. Neuropath. Exp. Neurol. 72(11): 1062-71 (2013), which is hereby incorporated by reference in its entirety).

In another embodiment, the AAV vector is one that has been engineered or selected for its enhanced CNS transduction after intraparenchymal administration, e.g., AAV-DJ (Grimm et al., J. Viol. 82:5887-5911 (2008), which is hereby incorporated by reference in its entirety); increased transduction of neural stem and progenitor cells, e.g., SCH9 and AAV4.18 (Murlidharan et al., J. Virol. 89: 3976-3987 (2015) and Ojala et al., Mol. Ther. 26:304-319 (2018), which are hereby incorporated by reference in their entirety); enhanced retrograde transduction, e.g., rAAV2-retro (Muller et al., Nat. Biotechnol. 21:1040-1046 (2003), which is hereby incorporated by reference in its entirety); selective transduction into brain endothelial cells, e.g., AAV-BRI (Korbelin et al., EMBO Mol. Med. 8: 609-625 (2016), which is hereby incorporated by reference in its entirety); or enhanced transduction of the adult CNS after IV administration, e.g., AAV-PHP.B and AAVPHP.eB (Deverman et al., Nat. Biotechnol. 34: 204-209 (2016) and Chan et al., Nat. Neurosci. 20: 1172-1179 (2017), which are hereby incorporated by reference in their entirety.

In accordance with this embodiment, the expression vector construct encoding the CEMIP antibody-based molecule can include the polynucleotide sequence encoding the heavy chain polypeptide, a fragment thereof, a variant thereof, or combinations thereof. The expression construct can also include a polynucleotide sequence encoding the light chain polypeptide, a fragment thereof, a variant thereof, or combinations thereof.

The expression construct also typically comprises a promoter sequence suitable for driving expression of the CEMIP antibody-based molecule. Suitable promoter sequences include, without limitation, the elongation factor 1-alpha promoter (EF1a) promoter, a phosphoglycerate kinase-1 promoter (PGK) promoter, a cytomegalovirus immediate early gene promoter (CMV), a chimeric liver-specific promoter (LSP), a cytomegalovirus enhancer/chicken beta-actin promoter (CAG), a tetracycline responsive promoter (TRE), a transthyretin promoter (TTR), a simian virus 40 promoter (SV40) and a CK6 promoter. Other promoters suitable for driving gene expression in mammalian cells that are known in the art are also suitable for incorporation into the expression constructs disclosed herein.

The expression construct can further encode a linker sequence. The linker sequence can encode an amino acid sequence that spatially separates and/or links the one or more components of the expression construct (heavy chain and light chain components of the encoded antibody).

Another aspect of the present disclosure is directed to a host cell comprising the vectors described herein. The CEMIP antibody-based molecule described herein can be optionally produced by a cell line, a mixed cell line, an immortalized cell or clonal population of immortalized cells, as well known in the art (see e.g., Ausubel et al., ed., Current Protocols in Molecular Biology, John Wiley & Sons, Inc., NY, N.Y. (1987-2001); Sambrook et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan et al., eds., Current Protocols in Immunology, John Wiley & Sons, Inc., NY (1994-2001); Colligan et al., Current Protocols in Protein Science, John Wiley & Sons, NY, N.Y., (1997-2001), which are hereby incorporated by reference in their entirety).

In any embodiment, the host cell chosen for expression may be of mammalian origin. Suitable mammalian host cells include, without limitation, COS-1 cells, COS-7 cells, HEK293 cells, BHK21 cells, CHO cells, BSC-1 cells, HeG2 cells, SP2/0 cells, HeLa cells, mammalian myeloma cells, mammalian lymphoma cells, or any derivative, immortalized or transformed cell thereof. Other suitable host cells include, without limitation, yeast cells, insect cells, and plant cells. Alternatively, the host cell may be selected from a species or organism incapable of glycosylating polypeptides, e.g., a prokaryotic cell or organism, such as BL21, BL21(DE3), BL21-GOLD(DE3), XL1-Blue, JM109, HMS174, HMS174(DE3), and any of the natural or engineered E. coli spp, Klebsiella spp., or Pseudomonas spp strains.

The CEMIP antibody-based molecules described herein can be prepared by any of a variety of techniques using the isolated polynucleotides, vectors, and host cells described supra. In general, antibodies can be produced by cell culture techniques, including the generation of monoclonal antibodies via conventional techniques, or via transfection of antibody genes, heavy chains and/or light chains into suitable bacterial or mammalian cell hosts, in order to allow for the production of antibodies, wherein the antibodies may be recombinant. Standard molecular biology techniques are used to prepare the recombinant expression vector, transfect the host cells, select for transformants, culture the host cells and recover the antibody from the culture medium. Transfecting the host cell can be carried out using a variety of techniques commonly used for the introduction of exogenous DNA into a prokaryotic or eukaryotic host cell, e.g., by electroporation, calcium-phosphate precipitation, DEAE-dextran transfection and the like. Although it is possible to express the antibodies described herein in either prokaryotic or eukaryotic host cells, expression of antibodies in eukaryotic cells is sometimes preferable, and sometimes preferable in mammalian host cells, because such eukaryotic cells (and in particular mammalian cells) are more likely than prokaryotic cells to assemble and secrete a properly folded and immunologically active antibody.

As noted above, exemplary mammalian host cells for expressing the recombinant antibodies disclosed herein include Chinese Hamster Ovary (CHO cells) (including dhfr-CHO cells, described in Urlaub and Chasin, Proc. Natl. Acad. Sci. USA, 77: 4216-4220 (1980), which is hereby incorporated by reference in its entirety). Other suitable mammalian host cells include, without limitation, NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown.

Host cells can also be used to produce functional antibody fragments, such as Fab fragments or scFv molecules. It is understood that variations on the above procedure are within the scope of the present disclosure. For example, it may be desirable to transfect a host cell with DNA encoding functional fragments of either the light chain and/or the heavy chain of an antibody described herein. Recombinant DNA technology may also be used to remove some or all of the DNA encoding either or both of the light and heavy chains that is not necessary for binding to the antigens of interest. The molecules expressed from such truncated DNA molecules are also encompassed by the antibodies described herein.

The antibodies and antibody binding fragments are recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

The CEMIP antibody or binding fragments described herein can also be coupled to a detectable label for utilization as a diagnostic antibody reagent. The label can be any detectable moiety known and used in the art. Suitable labels include, without limitation, radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

Detecting the presence of CEMIP proteins or peptides using the diagnostic antibody reagent of the present application can be carried out in vitro or in vivo using in vivo imaging techniques. In vivo imaging involves administering to a subject a labeled CEMIP antibody-based molecule described herein, and detecting the binding of the CEMIP antibody-based molecule thereof to the CEMIP protein in vivo.

In any embodiment, the CEMIP antibody-based molecule is a radiolabeled anti-CEMIP antibody or CEMIP- or anti-CEMIP-bound nanoparticle conjugated to an anti-CEMIP antibody.

Suitable radionuclides for use in labelling anti-CEMIP antibodies include, without limitation, ⁸⁶Re, ⁹⁰Y, ⁶⁷Cu, ¹⁶⁹Er, ¹²¹Sn, ¹²⁷Te, ¹⁴²Pr, ¹⁴³Pr, ¹⁹⁸AU, ¹⁹⁹Au, ¹⁶¹Tb, ¹⁰⁹Pd, ¹⁸⁸Rd, ¹⁶⁶Dy, ¹⁶⁶Ho, ¹⁴⁹Pm, ¹⁵¹Pm, ¹⁵³Sm, ¹⁵⁹Gd, ¹⁷²Tm, ¹⁶⁹Yb, ¹⁷⁵Yb, ¹⁷⁷Lu, ¹⁰⁵Rh, ¹¹¹Ag, ¹³¹I, ¹⁷⁷mSn, ²²⁵Ac, ²²⁷Th, ²¹¹At, and combinations thereof.

Procedures for labeling antibodies with radioactive isotopes are generally known in the art. For example, there are a wide range of moieties which can serve as chelating ligands and which can be derivatized to an CEMIP antibody-based molecule. Procedures for iodinating biological agents, such as antibodies, and binding portions thereof, are described by Hunter and Greenwood, “Preparation of Iodine-131 Labelled Human Growth Hormone of High Specific Activity,” Nature 144:496-496 (1962), David et al., “Protein Iodination With Solid State Lactoperoxidase,” Biochemistry 13:1014-1021 (1974), and U.S. Pat. No. 3,867,517 to Ling and U.S. Pat. No. 4,376,110 to David, which are hereby incorporated by reference in their entirety. Other procedures for iodinating biological agents are described by Greenwood et al., “The Preparation of I-131-Labelled Human Growth Hormone of High Specific Radioactivity,” Biochem. J. 89:114-123 (1963); Marchalonis, “An Enzymatic Method for the Trace Iodination of Immunoglobulins and Other Proteins,” Biochem. J. 113:299-305 (1969); and Morrison et al., “Use of Lactoperoxidase Catalyzed Iodination in Immunochemical Studies,” Immunochemistry 8:289-297 (1971), which are hereby incorporated by reference in their entirety. Procedures for 99mTc-labeling are described by Rhodes, B. et al. in Burchiel, S. et al. (eds.), Tumor Imaging: The Radioimmunochemical Detection of Cancer, New York: Masson 111-123 (1982) and the references cited therein, which are hereby incorporated by reference in their entirety. Procedures suitable for 111 In-labeling biological agents are described by Hnatowich et al., “The Preparation of DTPA-coupled Antibodies Radiolabeled With Metallic Radionuclides: an Improved Method,” J. Immunol. Methods 65:147-157 (1983), Hnatowich et al., “Coupling Antibody With DTPA—an Alternative to the Cyclic Anhydride,” Int. J. Applied Radiation (1984), and Buckley et al., “An Efficient Method For Labelling Antibodies With 111In,” F.E.B.S. 166:202-204 (1984), which are hereby incorporated by reference in their entirety.

Diagnostic CEMIP antibody-based molecules can be administered by intravenous injection into the body of a patient, or directly into the brain by intracranial injection or by drilling a hole through the skull. The dosage of antibody should be within the same ranges as for treatment methods. In accordance with this embodiment, the CEMIP antibody-based molecule is coupled to an imaging agent to facilitate in vivo imaging. The imaging agent can be any agent known to one of skill in the art to be useful for imaging, preferably being a medical imaging agent. Examples of medical imaging agents include, but are not limited to, single photon emission computed tomography (SPECT) agents, positron emission tomography (PET) agents, magnetic resonance imaging (MRI) agents, nuclear magnetic resonance imaging (NMR) agents, x-ray agents, optical agents (e.g., fluorophores, bioluminescent probes, near infrared dyes, quantum dots), ultrasound agents and neutron capture therapy agents, computer assisted tomography agents, two photon fluorescence microscopy imaging agents, and multi-photon microscopy imaging agents. Exemplary detectable markers include radioisotopes (e.g., ¹⁸F, ¹¹C, ¹³N, ⁶⁴Cu, ¹²⁴I, 76Br, ⁸²Rb, ⁶⁸Ga, ⁹⁹mTc, ¹¹¹In, ²⁰¹Tl or ¹⁵O, which are suitable for PET and/or SPECT use) and ultra-small superparamagnetic particles of iron oxide (USPIO) which are suitable for MRI.

### Pharmaceutical Compositions Comprising CEMIP Antibody-Based Molecules

The CEMIP antibody-based molecules or polynucleotide encoding the CEMIP antibody-based molecules disclosed herein are advantageously administered as pharmaceutical compositions comprising an active therapeutic agent (i.e., the CEMIP antibody) and one or more of a variety of other pharmaceutically acceptable components. See REMINGTON: THE SCIENCE AND PRACTICE OF PHARMACY (21^{st} Edition) (2005) (Troy, D. B. et al. (Eds.) Lippincott Williams & Wilkins (Pubis.), Baltimore MD), which is hereby incorporated by reference in its entirety. The preferred form depends on the intended mode of administration and therapeutic application. The compositions can also include, depending on the formulation desired, pharmaceutically acceptable, non-toxic carriers, excipients, diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition, and which are vehicles commonly used to formulate pharmaceutical compositions for animal or human administration. The diluent is selected to not affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, or non-toxic, nontherapeutic, non-immunogenic stabilizers and the like. Examples of suitable aqueous and non-aqueous carriers which may be employed in the pharmaceutical compositions disclosed herein include water, saline, phosphate-buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well-known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present disclosure is contemplated.

The compositions may also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Suitability for carriers and other components of pharmaceutical compositions is determined based on the lack of significant negative impact on the desired biological properties of the active antibody-based molecule of the present disclosure (e.g., less than a substantial impact (e.g., 10% or less relative inhibition, 5% or less relative inhibition, etc.) on antigen binding).

The pharmaceutical compositions of the present disclosure may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present disclosure may also comprise isotonicity agents, such as sugars, polyalcohols, such as mannitol, sorbitol, glycerol or sodium chloride in the compositions.

The pharmaceutical compositions of the present disclosure may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The CEMIP antibody-based molecule of the present disclosure may be prepared with carriers that will protect the antibody-based molecule against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well-known in the art. Methods for the preparation of such formulations are generally known to those skilled in the art. See, e.g., SUSTAINED AND CONTROLLED RELEASE DRUG DELIVERY SYSTEMS, J. R. Robinson, ed., Marcel Dekker, Inc., New York, 1978.

In any embodiment, the CEMIP antibody-based molecule of the present disclosure is formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art.

Pharmaceutical compositions for injection must typically be sterile and stable under the conditions of manufacture and storage. The composition may be formulated as a solution, microemulsion, liposome, or other ordered structure suitable to achieve high drug concentration. The carrier may be an aqueous or non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

For parenteral administration, CEMIP antibody-based molecules of the present disclosure are typically formulated as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. CEMIP antibody-based molecule of the disclosure can be administered in the form of a depot injection or implant preparation which can be formulated in such a manner as to permit a sustained release of the active ingredient. An exemplary composition comprises an scFv at about 5 mg/mL, formulated in aqueous buffer consisting of 50 mM L-histidine, 150 mM NaCl, adjusted to pH 6.0 with HCl.

Typically, compositions are thus prepared as injectables, either as liquid solutions or suspensions; solid forms suitable for solution in, or suspension in, liquid vehicles prior to injection can also be prepared. The preparation also can be emulsified or encapsulated in liposomes or micro particles, such as polylactide, polyglycolide, or copolymer, for enhanced adjuvant effect (Langer, et al., Science 249:1527 (1990); Hanes, et al., Advanced Drug Delivery Reviews 28:97-119 (1997), which are hereby incorporated by reference in their entirety). Additional formulations suitable for other modes of administration include oral, intranasal, and pulmonary formulations, suppositories, and transdermal applications.

### Administration of the Pharmaceutical Compositions Comprising CEMIP Antibody-Based Molecules

The CEMIP antibody-based molecules of the present disclosure can be administered by parenteral, topical, oral or intranasal means for therapeutic treatment. Intramuscular injection (for example, into the arm or leg muscles) and intravenous infusion are preferred methods of administration of the molecules of the present disclosure. In some methods, such molecules are administered as a sustained release composition or device, such as a Medipad™ device (Elan Pharm. Technologies, Dublin, Ireland). In some methods, the molecules of the present disclosure are injected directly into a particular tissue where deposits have accumulated, for example intracranial injection.

In one embodiment, a pharmaceutical composition of the present disclosure is administered parenterally. The phrases “parenteral administration” and “administered parenterally” as used herein denote modes of administration other than enteral and topical administration, usually by injection, and include epidermal, intravenous, intramuscular, intraarterial, intrathecal, intracapsular, intracranial, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection, subcutaneous and infusion. In any embodiment, that pharmaceutical composition comprising CEMIP antibody-based molecules is administered by intravenous or subcutaneous injection or infusion.

In therapeutic applications (i.e., in applications involving a patient having a primary tumor that is at risk of metastasizing to the brain, a patient having an autoimmune disorder, or a patient having an inflammatory condition) the CEMIP antibody-based molecules of the present disclosure are administered to such patient in an amount sufficient to cure, treat, or at least partially arrest, the symptoms of the disease (as adduced by biochemical, histologic and/or behavioral assessment), including its complications and intermediate pathological phenotypes in development of the disease. In any embodiment, the administration of the t CEMIP antibody-based molecule of the present disclosure reduces or eliminates the disorder.

Effective doses of the provided therapeutic molecules of the present disclosure, for the treatment of the above-described conditions may vary depending upon many different factors, including means of administration, target site, physiological state of the patient, other medications administered. Treatment dosages are typically titrated to optimize their safety and efficacy. On any given day that a dosage is given, the dosage of the CEMIP antibody-based molecules as described herein may range from about 0.0001 to about 100 mg/kg, and more usually from about 0.01 to about 5 mg/kg, of the patient's body weight. For example, dosages can be 1 mg/kg body weight or 10 mg/kg body weight or within the range of 1-10 mg/kg body weight. Exemplary dosages thus include: from about 0.1 to about mg/kg body weight, from about 0.1 to about 5 mg/kg body weight, from about 0.1 to about 2 mg/kg body weight, from about 0.1 to about 1 mg/kg body weight, for instance about 0.15 mg/kg body weight, about 0.2 mg/kg body weight, about 0.5 mg/kg body weight, about 1 mg/kg body weight, about 1.5 mg/kg body weight, about 2 mg/kg body weight, about 5 mg/kg body weight, or about 10 mg/kg body weight

A physician or veterinarian having ordinary skill in the art may readily determine and prescribe the effective amount of the pharmaceutical composition required. For example, the physician or veterinarian could start doses of antibody-based molecule in the pharmaceutical composition at levels lower than that required in order to achieve the desired therapeutic effect and gradually increase the dosage until the desired effect is achieved. In general, a suitable daily dose of a composition of the present disclosure will be that amount of the CEMIP antibody-based molecule which is the lowest dose effective to produce a therapeutic effect. Such an effective dose will generally depend upon the factors described above. Administration may e.g. be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible the antibody-based molecule of the present disclosure to be administered alone, it is preferable to administer the antibody-based molecule as a pharmaceutical composition as described above.

For therapeutic purposes, the CEMIP antibody-based molecules of the present disclosure are usually administered on multiple occasions. Intervals between single dosages (e.g., a bolus or infusion) can be weekly, monthly, or yearly. In some methods, dosage is adjusted to achieve a plasma concentration of 1-1000 μg/mL and in some methods 25-300 μg/mL. Alternatively, the therapeutic molecules of the present disclosure can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and non-human antibodies. scFv molecules generally have short serum half-lives.

In another embodiment, a pharmaceutical composition comprising a recombinant nucleic acid sequence encoding the CEMIP antibody-based molecule as described herein, is administered to a subject to facilitate in vivo expression and formation of the antibody-based molecule for the treatment of conditions mediated by CEMIP as described herein. Expression vector constructs suitable for use in this embodiment of the disclosure are described supra.

The polynucleotide compositions can result in the generation of the CEMIP antibody-based molecule in the subject within at least about 1 hour, 2 hours, 3 hours, 4 hours, hours, 6 hours, 7 hours, 8 hours, 9 hours, 10 hours, 11 hours, 12 hours, 13 hours, 14 hours, hours, 20 hours, 25 hours, 30 hours, 35 hours, 40 hours, 45 hours, 50 hours, or 60 hours of administration of the composition to the subject. The composition can result in generation of the antibody-based molecule in the subject within at least about 1 day, 2 days, 3 days, 4 days, days, 6 days, 7 days, 8 days, 9 days, or 10 days of administration of the composition to the subject. The composition can result in generation of the antibody-based molecule in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the persistent generation of the antibody-based molecule in the subject. The composition can result in the generation of the antibody-based molecule in the subject for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

### Therapeutic Utility of the CEMIP Antibody-Based Molecules

One aspect of the present disclosure relates to a method of increasing cell migration-inducing and hyaluronan-binding protein (CEMIP) signaling in a subject in need thereof. This method involves administering to the subject in need, a CEMIP antibody-based molecule as described herein, or a pharmaceutical composition comprising a CEMIP antibody-based molecule as described herein or a polynucleotide encoding a CEMIP antibody-based molecule as described herein. In accordance with this method, the composition is administered in an amount effective to decrease and/or inhibit CEMIP activity or function in the subject relative to CEMIP activity or function in the subject prior to said administering.

In any embodiment, such administration may be provided to a subject having a primary tumor that is at risk for the tumor metastasizing to the brain. Exemplary cancers that metastasize to the brain, and thus are suitable for treatment in accordance with the methods described herein include, without limitation, breast tumors, lung tumors, melanoma, renal tumors, colorectal tumors, esophageal tumors, small intestine tumors, stomach tumors, bladder tumors, liver tumors, pancreatic tumors, and prostate tumors.

In any embodiment, such administration may be provided to a subject having an autoimmune condition. Suitable autoimmune conditions that can be treated with the CEMIP antibodies described herein include, without limitation rheumatoid arthritis, multiple sclerosis, Sjogren's syndrome, systemic lupus erythematosus, autoimmune hepatitis, autoimmune thyroiditis, hemophagocytic syndrome (hemophagocytic lymphohistiocytosis), diabetes mellitus type 1, Crohn's condition, ulcerative colitis, psoriasis, psoriatic arthritis, idiopathic thrombocytonpenic pupura, polymyositis, dermatomyositis, myasthenia gravis, autoimmune thryroiditis, Evan's syndrome, autoimmune hemolytic anemia, aplastic anemia, autoimmune neutropenia, scleroderma, Reiter's syndrome, ankylosing spondylitis, pemphnigus, pemphigoid or autoimmune hepatitis, Behcet's condition, Celiac condition, Chagas condition, acute disseminated encephalomyelitis, Addison's condition, antiphospholipid antibody syndrome, autoimmune inner ear condition, bullous pemphigoid, Chronic obstructive pulmonary condition, Goodpasture's syndrome, Graves' condition, Guillain-Barré syndrome, Hashimoto's thyroditis, Hidradenitis suppurativa, Interstitial cystitis, neuromyotonia, pemphigus vulgaris, pernicious anemia, primary biliary cirrhosis, and vasculitis syndromes.

In any embodiment, such administration may be provided to a subject having an inflammatory condition. Suitable inflammatory conditions for treatment in accordance with the methods described herein include both chronic and acute inflammatory conditions. Inflammatory conditions that can be treated with the CEMIP antibody-based molecules described herein include, without limitation, allergic rhinitis, asthma, atopic eczema, coronary artery condition, peripheral artery condition, atherosclerosis, retinitis, pancreatitis, pericarditis, colitis, glomerulonephritis, lung inflammation, esophagitis, gastritis, duodenitis, ileitis, encephalomyelitis, transverse myelitis, cystitis, urethritis, mucositis, lymphadenitis, hepatitis, osteomyelitis, herpes zoster, dermatitis such as psoriasis, irritant dermatitis, seborrheic dermatitis, atopic dermatitis (eczema), allergic contact dermatitis, heat-induced skin Inflammation, drug-induced dermatitis, sweaty dermatitis, hives, autoimmune dermatitis, skin cancer such as melanoma, and bullous dermatitis, meningitis (i.e., inflammation of the protective membrane covering the brain and spinal cord), myelitis, encaphaloymyelitis (e.g., myalgic encephalomyelitis), acute disseminated encephalomyelitis, disseminated encephalomyelitis (encephalomyelitis disseminata), multiple sclerosis, autoimmune encephalomyelitis, arachnoiditis (i.e., surrounding nerves of the central nervous system), inflammation of the arachnoid membrane which is one of the protective membranes), granulomas, drug-induced inflammation or meningitis, Alzheimer's disease, stroke, HIV-dementia, Sly syndrome, CMT, retinopathy, feeling Parasitic infections, inflammatory demyelinating disorders, sensoriuenural hearing loss, spinal muscular atrophy, ALS, encephalitis, e.g., viral encephalitis and bacterial encephalitis, inflammatory lung disease, atopic asthma, non-atopic asthma, allergic asthma, atopic bronchial IgE-mediated asthma, bronchial asthma, essential asthma, true Asthma, intrinsic asthma caused by pathophysiological disturbances, extrinsic asthma caused by environmental factors, essential asthma of unknown or unapparent cause, non-atopic asthma, bronchitis asthma, emphysematous asthma, exercise induction Asthma, allergen-induced asthma, cold-induced asthma, occupational asthma, infectious asthma caused by bacterial, fungal, protozoan, or viral infections, nonallergic asthma, early asthma, wheezing infant syndrome, and bronchioles, bronchiolytis, chronic or acute bronchoconstriction, chronic bronchitis, small airway obstruction, and emphysema, include obstructive or inflammatory airway diseases such as chronic eosinophilic pneumonia, chronic obstructive pulmonary disease (COPD), chronic bronchitis associated with or not associated with COPD, emphysema, or dyspnea, bronchitis, e.g., acute bronchitis, acute larynx Tracheobronchitis, arachidin bronchitis, catarrhal bronchitis, croupus bronchitis, dry bronchitis, infectious asthmatic bronchitis, productive bronchitis, staphylococci or streptococci Acute lung injury, such as bronchitis and vesicular bronchitis, and bronchiectasis, such as columnar bronchiectasis, sacculated bronchiectasis, spindle bronchiectasis, capillary bronchiectasis (capillary bronchiectasis), cystic bronchiectasis, dry bronchiectasis, and follicular bronchiectasis, myocarditis, pericarditis, occlusive disease, atherosclerosis, myocardial infarction, thrombosis, autoimmune enteropathy, cardiomyopathy, Kawasaki disease, Juvenile idiopathic arthritis, Wegner's granulomatosis, Takayasu's arteritis, Kawasaki syndrome, anti-factor VIII autoimmune disease, necrotizing small vessel vasculitis, microscopic polyangiitis, Churg-Strauss syndrome, pauci-immune type nest Necrotizing glomerulonephritis, half-moon glomerulonephritis, antiphospholipid antibody syndrome, antibody-induced heart failure, thrombocytopenic purpura, autoimmune hemolytic anemia.

The term “treatment” or “treating” as used herein means ameliorating, slowing or reversing the progress or severity of a disease or disorder, or ameliorating, slowing or reversing one or more symptoms or side effects of such disease or disorder. For purposes of this disclosure, “treatment” or “treating” further means an approach for obtaining beneficial or desired clinical results, where “beneficial or desired clinical results” include, without limitation, alleviation of a symptom, diminishment of the extent of a disorder or disease, stabilized (i.e., not worsening) disease or disorder state, delay or slowing of the progression a disease or disorder state, amelioration or palliation of a disease or disorder state, and remission of a disease or disorder, whether partial or total, detectable or undetectable.

An “effective amount,” of the antibody-based molecule refers to an amount sufficient, at dosages and for periods of time necessary, to achieve an intended biological effect or a desired therapeutic result including, without limitation, clinical results. The phrase “therapeutically effective amount” when applied to an antibody-based molecule of the disclosure is intended to denote an amount of the antibody that is sufficient to ameliorate, palliate, stabilize, reverse, slow or delay the progression of a disorder or disease state, or of a symptom of the disorder or disease. In an embodiment, the method of the present disclosure provides for administration of the antibody-based molecule in combinations with other compounds. In such instances, the “effective amount” is the amount of the combination sufficient to cause the intended biological effect.

Another aspect of the present disclosure relates to a method of treating or inhibiting brain metastasis in a subject. This method involves administering, to a subject having a primary tumor, a CEMIP antibody-based molecule of the present disclosure in an amount effect to treat or inhibit brain metastasis in the subject.

Another aspect of the present disclosure relates to a method of treating or inhibiting autoimmune conditions in a subject. This method involves administering, to a subject having an autoimmune condition, a CEMIP antibody of the present disclosure in an amount effect to treat or inhibit the autoimmune condition in the subject.

Another aspect of the present disclosure relates to a method of treating or inhibiting inflammatory conditions in a subject. This method involves administering, to a subject having an inflammatory condition, a CEMIP antibody of the present disclosure in an amount effect to treat or inhibit the inflammatory condition in the subject.

### EXAMPLES

The following examples are provided to illustrate embodiments of the present application, but they are by no means intended to limit its scope.

### Example 1—Generation and Validation of CEMIP/KIAA1199 Neutralizing Antibodies and Testing their Targeting Specificity

A series of anti-human CEMIP antibodies was generated in mice and screened. Validation was performed on two human cell lines with high cellular and exosomal KIAA1199 (CEMIP); the gastric cell line MKN45 and the N2LA lung cancer cell line.

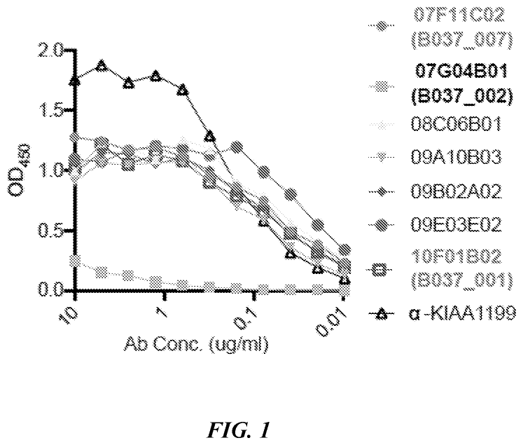

Antibodies targeting KIAA1199/CEMIP were generated in Balb/c and A/J mice. Immunogens included a truncated version of KIAA1199/CEMIP protein (amino acids 1-649) and plasmid DNA. DNA immunization is a strategy that is often successful for challenging or problematic antigens such as membrane-associated proteins, multi-pass membrane proteins or large proteins. DNA immunization with a high volume of CEMIP-encoding plasmid permits in vivo antigen production, bypassing immunogen (e.g. peptides and recombinant proteins) synthesis and purification. In this strategy, CEMIP is expressed in vivo by liver cells, and the protein maintains the native structures and goes through appropriate post-translational modifications. These properties contribute to the generation of antibodies binding to the native conformation of the target antigen, which is a crucial feature for developing therapeutic antibodies. After multiple rounds of injections, splenocytes were harvested and fused with myeloma cells to generate hybridoma. Hybridoma supernatants were tested by ELISA using purified KIAA1199/CEMIP protein (aa 1-649). Hybridoma from positive hits (10-fold over background) were subcloned, followed by purification of the monoclonal antibodies from hybridoma supernatant. From this initial phase, 7 anti-KIAA1199/CEMIP antibodies identified were tested by ELISA using purified KIAA1199/CEMIP protein (aa 1-649) (FIG. 1).

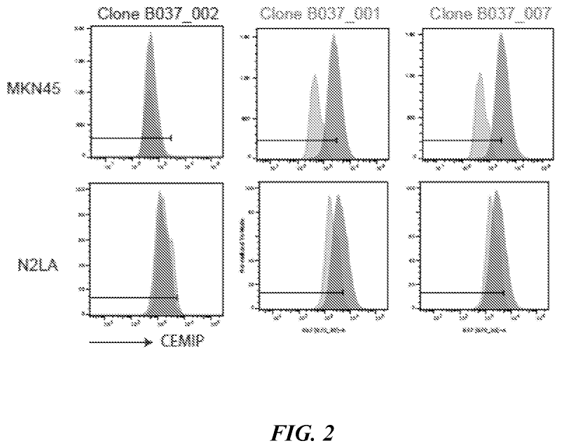

Purified monoclonal antibodies were tested in secondary flow-cytometry screens on two CEMIPhi cell lines, MKN45 and N2LA (FIG. 2). Flow cytometry is performed on live cells, and antibodies bind to the native cell surface CEMIP/KIAA1199. Three antibody clones with high affinity and specificity for human KIAA1199/CEMIP were identified from the first screen: 07F11C02, 10F01B02 and 01F11A01, and the sequence of these antibodies was obtained (Table 5). One of these clones resulted from the protein immunization and two resulted from the DNA immunization (FIG. 2). Clone 07G04B01 demonstrated no binding to KIAA1199/CEMIP and was used as a negative control in future experiments. These antibodies were tested for their ability to efficiently block brain-tropic tumor cell outgrowth in vitro, in organotypic cultures as described herein.

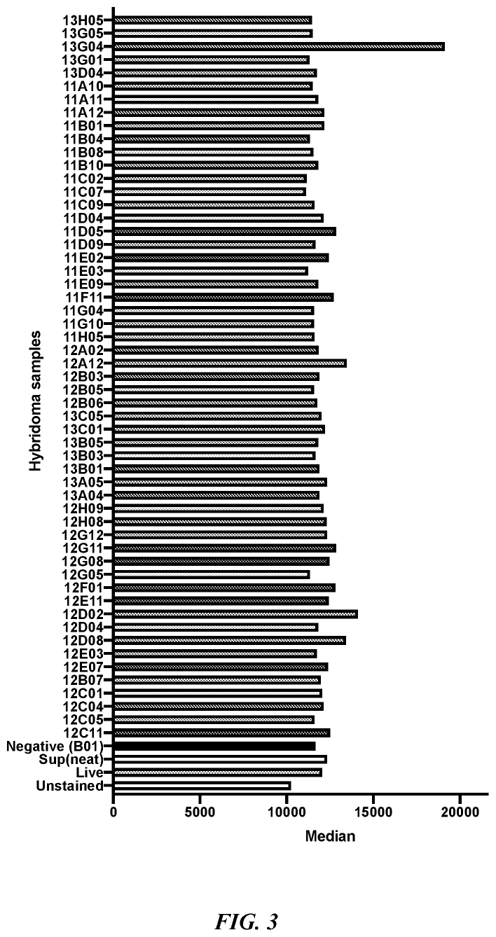

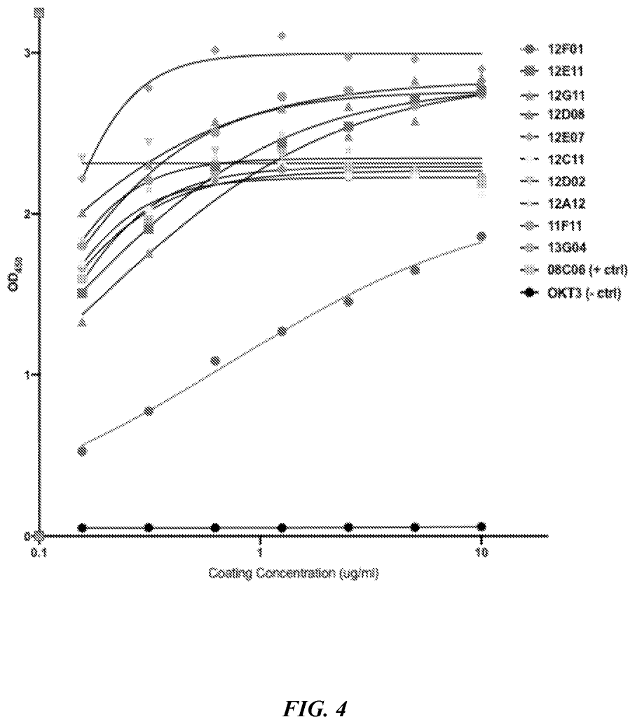

In the second phase of the screen, an additional 55 antibodies that bind to recombinant KIAA1199/CEMIP (aa 1-649) in ELISA assays were identified and tested by flow cytometry of exhausted hybridoma supernatants on the MKN45 cell line. These hybridomas were selected from semi-solid medium using a ClonePix instrument, resulting in monoclonality without the need for subcloning. Supernatant production from hybridomas producing binders was scaled up and antibodies were purified, tested and titrated by flow cytometry. Of the 55 clones screened, 29 were found to bind at levels higher than the negative control antibody (07G04B01) and of these 29, 13 clones, 13G04, 11D05, 11E02, 11F11, 12A12, 12G11, 12E11, 12G08, 12F01, 12D02, 12D08, 12E07, 12C11 were found to bind at levels higher than the positive control antibody (10F01B02), and were selected for further characterization (FIG. 3). These 13 purified antibodies as well as additional controls were re-screened by ELISA versus known KIAA1199-binding antibody and an irrelevant control (FIG. 4). The 4 antibodies with the highest anti-CEMIP/KIAA1199 binding based on these 2 assays, 12D02, 12D08, 12A12 and 13G04, were sequenced (Table 5).

### Example 2—CEMIP/KIAA1199 Antibody Binding Specificity

Numerous KIAA1199 (CEMIP) CRISPR KO clones in MKN45 gastric cells have been generated, which were screened first at the genomic level. Of six potential KO clones, 4 clones, KO6, KO11, KO13 and KO18 were confirmed as lacking cell surface CEMIP expression based on flow cytometry, which can be used to validate the specificity of anti-human CEMIP/KIAA1199 antibodies Interestingly, clone KO7 has a 50% reduction in the surface levels of CEMIP/KIAA1199 compared with the parental wild-type cell line, which may be useful in performing studies on neutralization efficiency of our antibodies at various levels (high, medium, low) of antigen expression. Thus, these clones and their exosomes can be used as negative controls on platforms testing the binding and neutralizing ability of anti-KIAA1199/CEMIP antibodies.

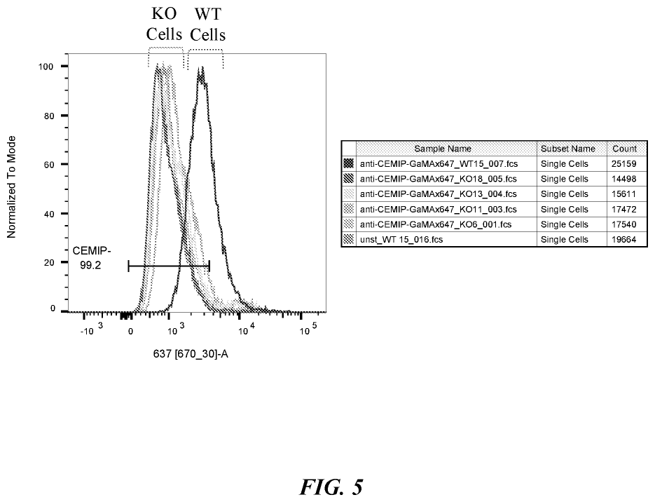

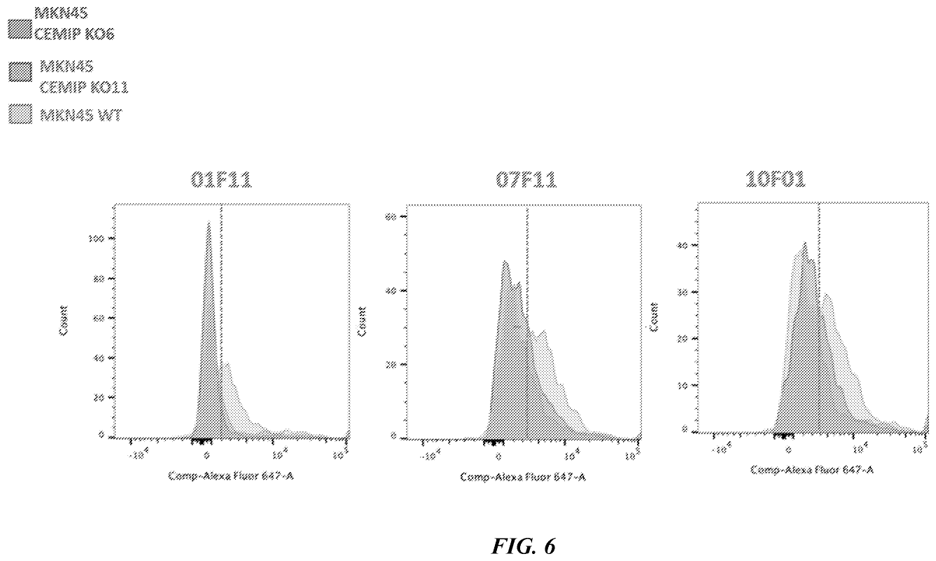

To test the binding specificity of anti-CEMIP antibodies disclosed herein, binding of the antibodies to native CEMIP expressed on the surface of wild-type human MKN45 gastric cell lines vs. CEMIP MKN45 KO lines generated using CRISPR/Cas9 was compared. Two micrograms of 10F01B02, 01F11A01, and 07F11C02 anti-CEMIP/KIAA1199 antibodies were used to test the binding to 2×10⁵ cells and binding was revealed with an Ax647-labelled goat anti-mouse secondary antibody (Biolegend). The flow cytometry data of FIG. 5 (10F01B02) and FIG. 6 (0F01B02, 01F11A01, 07F11C02) demonstrates that while these antibody clones bind robustly to wild-type MKN45 cells expressing high levels of CEMIP, they do not bind to either of the MKN45 CEMIP KO cell lines.

### Example 3—CEMIP Antibody Mediated Inhibition of Metastatic Breast Cancer Cell Survival in Brain Microenvironment

A previously described 3D organotypic brain slice assay (Rodrigues G et al, “Tumour Exosomal CEMIP Protein Promotes Cancer Cell Colonization in Brain Metastasis,” Nature Cell Biology, 21(11): 1403-12 (2019), which is hereby incorporated by reference in its entirety) was employed to test the capacity of each of the 10F01B02, 01F11A01, and 07F11C02 antibody clones to inhibit CEMIP function. This assay relies on the capacity of CEMIP-positive exosomes isolated from brain tropic. MDA-MB231 breast cancer cells (BrT1/Yoneda) to promote the growth of parental MDA-MB231, that otherwise lack the capacity to thrive in the brain microenvironment.

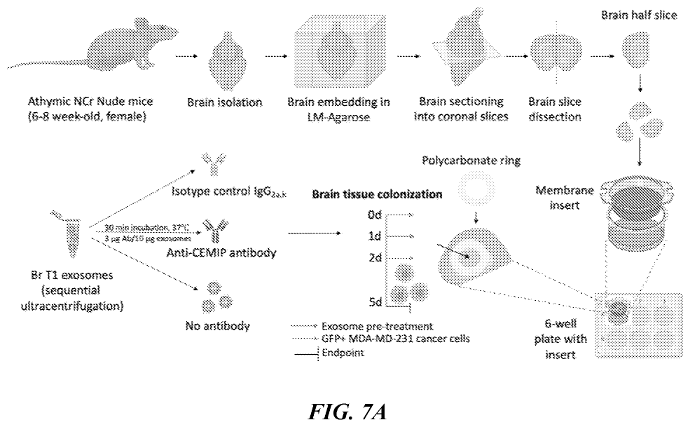

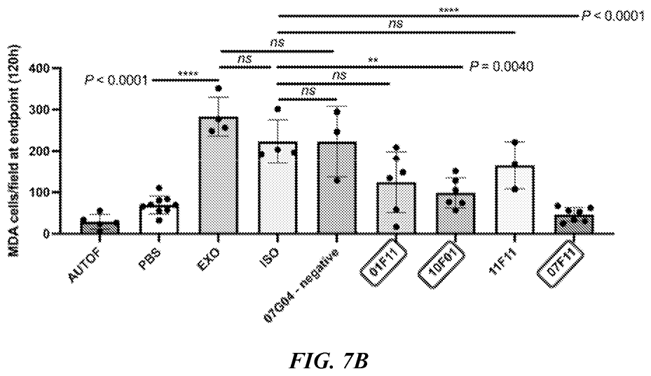

A schematic summarizing this organotypic brain slice assay is provided in FIG. 7A. In reference to FIG. 7A, brains are isolated from 6-8 week old female nude mice, embedded in low meting point agarose and sectioned using a Leica. Vibratome. The slices are placed in culture on inserts, and polycarbonate rings are placed on top of the slice to allow treatment with 5 ug of UMW-positive brain tropic exosomes twice, 24 hours apart. Control sections were treated with PBS only, IgG2a k isotype, and an antibody that does not bind to CEMIP as determined by both Elisa and flow assays (07G04). To test the capacity of the antibody clones described herein to inhibit CEMIP function, CEMIP-positive exosomes were incubated with 3 ug antibody/10 ug of exosomes for 30 min at 37° C. before being: applied inside the polycarbonate ring on the brain slice. Forty-eight hours post culture initiation and 24 h after the administration of the second dose of exosomes, GFP-labelled parental MDA-M8231 cells are applied inside the ring as previously described (Rodrigues G et al, “Tumour Exosomal CEMIP Protein Promotes Cancer Cell Colonization in Brain Metastasis,” Nature Cell Biology, 21(11): 1403-12 (2019), which is hereby incorporated by reference in its entirety).

As previously described. CEMIP-positive brain tropic exosome treatment promotes the survival and proliferation of GFP-labelled parental MDA-MB231 cells, resulting in a 3 to 5-fold increase in GFP⁺ cells on the slice compared to PBS treatment (see FIG. 7B, compare PBS and exosome (EXO) treatment and Rodrigues G et al, “Tumour Exosomal CEMIP Protein Promotes Cancer Cell Colonization in Brain Metastasis,” Nature Cell Biology, 21(11): 1403-12 (2019), which is hereby incorporated by reference in its entirety). Exosome incubation with IgG2a,k isotype and an antibody that does not bind to CENCIP as determined by both Elisa and flow assays (07G04) did not affect GFP⁺ cell growth resulting in similar numbers of cells on the slice at the endpoint (see FIG. 7B bars for EXO (exosome alone), ISO (IgG2a,k isotype) and 07(104 (non-CEMIP antibody). In contrast, pre-incubation of CEMIP-positive exosomes with 01F11, 07F11, or 10F01 antibodies resulted in significant growth inhibition, suggesting they interfered with CEMIP function (see FIG. 7B base for 01F11, 10F01, and 07F11). The strongest inhibition was observed with antibody clone 07F11, pre-treatment with which resulted in a 6-fold decrease in GFP+ cells on the slice, followed by 10F01 and 01F11, the latter resulting in a 2-fold decrease in GFP+ cells.

TABLE 5
Amino Acid Sequences of Exemplary
Antibodies of the Disclosure

| mAb/Fab | | | SEQ ID |
|---|---|---|---|
| name | Region | Sequence | NO: |
| cAb4853 | V_{H} | DVQLVESGGGLVQPGGSRKLSCAASGFAFSSFGMHWVRQAP | 44 |
| 07F11C02 | IgG2a | ERGLEWVAYISSASNTIYYADTVKGRFTISRDNPKSTLFLQ | |
| | | MTSLRSEDTAIYYCAKRDWDLYAMDYWGQGTSVTVSS | |
| | C_{H} | AKTTAPSVYPLAPVCGDTTGSSVTLGCLVKGYFPEPVTLTW | 72 |
| | | NSGSLSSGVHTFPAVLQSDLYTLSSSVTVTSSTWPSQSITC | |
| | | NVAHPASSTKVDKKIEPRGPTIKPCPPCKCPAPNLLGGPSV | |
| | | FIFPPKIKDVLMISLSPIVTCVVVDVSEDDPDVQISWFVNN | |
| | | VEVHTAQTQTHREDYNSTLRVVSALPIQHQDWMSGKEFKCK | |
| | | VNNKDLPAPIERTISKPKGSVRAPQVYVLPPPEEEMTKKQV | |
| | | TLTCMVTDFMPEDIYVEWTNNGKTELNYKNTEPVLDSDGSY | |
| | | FMYSKLRVEKKNWVERNSYSCSVVHEGLHNHHTTKSFSRTP | |
| | | GK | |
| | V_{L} | DIVMTQSQKFLSTSVGDRVTITCKASQNVGTAVAWYQQKPG | 45 |
| | kappa | QSPELLIYSASNRHTGVPARFTGSGSGTDFTLTITNMQSED | |
| | | LADYFCQQYSSSPTFGGGTKLESK | |
| | C_{L} | RADAAPTVSIFPPSSEQLTSGGASVVCFLNNFYPKDINVKW | 73 |
| | | KIDGSERQNGVLNSWTDQDSKDSTYSMSSTLTLTKDEYERH | |
| | | NSYTCEATHKTSTSPIVKSFNRNEC | |
| cAb4854 | V_{H} | DVQLVESGGGLVQPGGSRKLSCAASGFPFSSFGMHWVRQAP | 46 |
| 10F01B02 | IgG2a | DKGLEWVAYISSASNTIYYADTVKGRFTVSRDNPKNTLFLQ | |
| | | MTSLRSEDTAIYYCTKRNWDLYAMDYWGQGTSVTVSS | |
| | C_{H} | AKTTAPSVYPLAPVCGDTTGSSVTLGCLVKGYFPEPVTLTW | 74 |
| | | NSGSLSSGVHTFPAVLQSDLYTLSSSVTVTSSTWPSQSITC | |
| | | NVAHPASSTKVDKKIEPRGPTIKPCPPCKCPAPNLLGGPSV | |
| | | FIFPPKIKDVLMISLSPIVTCVVVDVSEDDPDVQISWFVNN | |
| | | VEVHTAQTQTHREDYNSTLRVVSALPIQHQDWMSGKEFKCK | |
| | | VNNKDLPAPIERTISKPKGSVRAPQVYVLPPPEEEMTKKQV | |
| | | TLTCMVTDFMPEDIYVEWTNNGKTELNYKNTEPVLDSDGSY | |
| | | FMYSKLRVEKKNWVERNSYSCSVVHEGLHNHHTTKSFSRTP | |
| | | GK | |
| | V_{L} | DIVMTQSQKFMSTSVGDRVSITCKANQNVGTAVAWYQQKPG | 47 |
| | kappa | QSPKLLIYSTSNRDTGVPDRFTGSGSGTDFTLTINYIQSED | |
| | | LADYFCQQYRNYPTFGGGTKLEIK | |
| | C_{L} | RADAAPTVSIFPPSSEQLTSGGASVVCFLNNFYPKDINVKW | 75 |
| | | KIDGSERQNGVLNSWTDQDSKDSTYSMSSTLTLTKDEYERH | |
| | | NSYTCEATHKTSTSPIVKSFNRNEC | |
| cAb4855 | V_{H} | QVTLKESGPGILQPSQTLSLTCSFSGFSLSTSGMGVGWIRQ | 48 |
| 01F11A01 | IgG2a | PSGKGLEWLAHIWWNDEKYYNAALKSRLTISKDTSKNQVFL | |
| | | KIASVDAADTATYYCARITGAWFPYWGQGTLVTVSA | |
| | C_{H} | AKTTAPSVYPLAPVCGDTTGSSVTLGCLVKGYFPEPVTLTW | 76 |
| | | NSGSLSSGVHTFPAVLQSDLYTLSSSVTVTSSTWPSQSITC | |
| | | NVAHPASSTKVDKKIEPRGPTIKPCPPCKCPAPNLLGGPSV | |
| | | FIFPPKIKDVLMISLSPMVTCVVVDVSEDDPDVQISWFVNN | |
| | | VEVLTAQTQTHREDYNSTLRVVSALPIQHQDWMSGKEFKCK | |
| | | VNNKDLPAPIERTISKPKGSVRAPQVYVLPPPEEEMTKKQV | |
| | | TLTCMVTDFMPEDIYVEWTNNGKTELNYKNTEPVLDSDGSY | |
| | | FMYSKLRVEKKNWVERNSYSCSVVHEGLHNHHTTKSFSRTP | |
| | | GK | |
| | V_{L} | DIVMTQSPATLSVTPGDRVSLSCRASQHISEYLHWYQQKSH | 49 |
| | kappa | ESPRLLIKYGSQSISGIPSRFSGSGSGSDFTLNINSVEPED | |
| | | VGVYYCQNGHSFPYTFGGGTKLEIK | |
| | C_{L} | RADAAPTVSIFPPSSEQLTSGGASVVCFLNNFYPKDINVKW | 77 |
| | | KIDGSERQNGVLNSWTDQDSKDSTYSMSSTLTLTKDEYERH | |
| | | NSYTCEATHKTSTSPIVKSFNRNEC | |
| cAb5775 | V_{H} | EVQLQQSGPELVKPGASMKISCKASGYSFTAYTMNWVKQSH | 50 |
| 12D02 | IgG1 | GENLEWIGLINPYNGGTTYNQKFKGKATLTVDKSSSTAYME | |
| | | LLSLTSEDSAVYYCASYDYGYAMDYWGQGTSVTVSS | |
| | C_{H} | AKTTPPSVYPLAPGSAAQTNSMVTLGCLVKGYFPEPVTVTW | 78 |
| | | NSGSLSSGVHTFPAVLQSDLYTLSSSVTVPSSTWPSETVTC | |
| | | NVAHPASSTKVDKKIVPRDCGCKPCICTVPEVSSVFIFPPK | |
| | | PKDVLTITLTPKVTCVVVDISKDDPEVQFSWFVDDVEVHTA | |
| | | QTQPREEQFNSTFRSVSELPIMHQDWLNGKEFKCRVNSAAF | |
| | | PAPIEKTISKTKGRPKAPQVYTIPPPKEQMAKDKVSLTCMI | |
| | | TDFFPEDITVEWQWNGQPAENYKNTQPIMDTDGSYFVYSKL | |
| | | NVQKSNWEAGNTFTCSVLHEGLHNHHTEKSLSHSPGK | |
| | V_{L} | DVLMTQTPLSLPVSLGDQASISCRSSQSIVHRNGNTYLEWY | 51 |
| | kappa | LQKPGQSPKLLIYKVSNRFSGVPDRFSGSGSGTDFTLKISR | |
| | | VEAEDLGVYYCFQGSHVPWTFGGGTKLEIK | |
| | C_{L} | RADAAPTVSIFPPSSEQLTSGGASVVCFLNNFYPKDINVKW | 79 |
| | | KIDGSERQNGVLNSWTDQDSKDSTYSMSSTLTLTKDEYERH | |
| | | NSYTCEATHKTSTSPIVKSFNRNEC | |
| cAb5776 | V_{H} | EVQLQQSGPELVKPGVSMKISCKASGYSFTAYTMNWVKQSH | 52 |
| 12A12 | IgG1 | GENLEWIGLINPYNGGTTYNQKFKGKATLTVDKSSSTAYME | |
| | | LLSLTSEDSAVYYCASYDYGYAMDYWGQGTSVTVSS | |
| | C_{H} | AKTTPPSVYPLAPGSAAQTNSMVTLGCLVKGYFPEPVTVTW | 80 |
| | | NSGSLSSGVHTFPAVLQSDLYTLSSSVTVPSSTWPSETVTC | |
| | | NVAHPASSTKVDKKIVPRDCGCKPCICTVPEVSSVFIFPPK | |
| | | PKDVLTITLTPKVTCVVVDISKDDPEVQFSWFVDDVEVHTA | |
| | | QTQPREEQFNSTFRSVSELPIMHQDWLNGKEFKCRVNSAAF | |
| | | PAPIEKTISKTKGRPKAPQVYTIPPPKEQMAKDKVSLTCMI | |
| | | TDFFPEDITVEWQWNGQPAENYKNTQPIMDTDGSYFVYSKL | |
| | | NVQKSNWEAGNTFTCSVLHEGLHNHHTEKSLSHSPGK | |
| | V_{L} | DVLMTQTPLSLPVSLGDQASISCRSSQSIVHRSGNTYLEWY | 53 |
| | kappa | LQKPGQSPKLLIYKVSNRFSGVPDRFSGSGSGTDFTLKISR | |
| | | VEAEDLGVYYCFQGSHVPWTFGGGTKLEIK | |
| | C_{L} | RADAAPTVSIFPPSSEQLTSGGASVVCFLNNFYPKDINVKW | 81 |
| | | KIDGSERQNGVLNSWTDQDSKDSTYSMSSTLTLTKDEYERH | |
| | | NSYTCEATHKTSTSPIVKSFNRNEC | |
| cAb5777 | VH | EVQLQQSGAELVKPGASVKLSCTASGFNIKDTYMHWVKQRP | 54 |
| 12D08 | IgG2a | EQGLIWIGNIDPANGHTKYAPKFQGKATITADTSSNTAYLQ | |
| | | LSSLTSEDTAVYYCARSNDYDVDFDYWGQGTTLTVSS | |
| | C_{H} | AKTTAPSVYPLAPVCGDTTGSSVTLGCLVKGYFPEPVTLTW | 82 |
| | | NSGSLSSGVHTFPAVLQSDLYTLSSSVTVTSSTWPSQSITC | |
| | | NVAHPASSTKVDKKIEPRGPTIKPCPPCKCPAPNLLGGPSV | |
| | | FIFPPKIKDVLMISLSPIVTCVVVDVSEDDPDVQISWFVNN | |
| | | VEVHTAQTQTHREDYNSTLRVVSALPIQHQDWMSGKEFKCK | |
| | | VNNKDLPAPIERTISKPKGSVRAPQVYVLPPPEEEMTKKQV | |
| | | TLTCMVTDFMPEDIYVEWTNNGKTELNYKNTEPVLDSDGSY | |
| | | FMYSKLRVEKKNWVERNSYSCSVVHEGLHNHHTTKSFSRTP | |
| | | GK | |
| | V_{L} | DIVMSQSPSSLAVSAGEKVTMSCKSSQSLLNSRTRKNYLAW | 55 |
| | kappa | YQQKPGQSPKLLIYWASTRESGVPDRFTGSGSGTDFSLTIS | |
| | | SVQAEDLAVYYCKQSYNLYTFGGGTKLEIK | |
| | C_{L} | RADAAPTVSIFPPSSEQLTSGGASVVCFLNNFYPKDINVKW | 83 |
| | | KIDGSERQNGVLNSWTDQDSKDSTYSMSSTLTLTKDEYERH | |
| | | NSYTCEATHKTSTSPIVKSFNRNEC | |
| cAb5778 | V_{H} | EVHLQQSGPELVKPGASMKISCKASGYSFTAYTMNWVKQSH | 56 |
| 13G04 | IgG1 | GKNLEWIGLINPYNGGTTYNQKFKGKATLTVDKSSSTAYME | |
| | | LLSLTSEDSAVYYCASYYGGRGWYFDVWGAGTSVTVSS | |
| | C_{H} | AKTTPPSVYPLAPGSAAQTNSMVTLGCLVKGYFPEPVTVTW | 84 |
| | | NSGSLSSGVHTFPAVLQSDLYTLSSSVTVPSSTWPSETVTC | |
| | | NVAHPASSTKVDKKIVPRDCGCKPCICTVPEVSSVFIFPPK | |
| | | PKDVLTITLTPKVTCVVVDISKDDPEVQFSWFVDDVEVHTA | |
| | | QTQPREEQFNSTFRSVSELPIMHQDWLNGKEFKCRVNSAAF | |
| | | PAPIEKTISKTKGRPKAPQVYTIPPPKEQMAKDKVSLTCMI | |
| | | TDFFPEDITVEWQWNGQPAENYKNTQPIMDTDGSYFVYSKL | |
| | | NVQKSNWEAGNTFTCSVLHEGLHNHHTEKSLSHSPGK | |
| | V_{L} | DIVLTQSPASLAVSLGQRATISCRASKSVSTSGYSYVHWYQ | 57 |
| | kappa | QKPGQPPKLLIYLASNLESGVPARFSGSGSGTDFTLNIHPV | |
| | | EEEDAATYYCQNSRELPYTFGGGTKLEMK | |
| | C_{L} | RADAAPTVSIFPPSSEQLTSGGASVVCFLNNFYPKDINVKW | 85 |
| | | KIDGSERQNGVLNSWTDQDSKDSTYSMSSTLTLTKDEYERH | |
| | | NSYTCEATHKTSTSPIVKSFNRNEC | |

TABLE 6
Nucleotide Sequences of Exemplary Antibodies
of the Disclosure

| mAb/Fab | | | SEQ ID |
|---|---|---|---|
| name | Region | Sequence | NO: |
| cAb4853 | V_{H} | GATGTGCAGCTGGTGGAGTCTGGGGGAGGCTTAGTGCAGC | 58 |
| 07F11C02 | | CTGGAGGGTCCCGGAAACTCTCCTGTGCAGCCTCTGGATT | |
| | | CGCTTTCAGTAGCTTTGGAATGCACTGGGTTCGTCAGGCT | |
| | | CCAGAGAGGGGGCTGGAGTGGGTCGCATACATTAGTAGTG | |
| | | CCAGCAATACCATCTACTATGCAGACACAGTGAAGGGCCG | |
| | | ATTCACCATCTCCAGAGACAATCCCAAGAGCACCCTGTTC | |
| | | CTGCAAATGACCAGTCTAAGGTCTGAGGACACGGCCATTT | |
| | | ATTACTGTGCAAAGCGAGACTGGGACCTCTATGCTATGGA | |
| | | CTACTGGGGTCAAGGAACCTCAGTCACCGTCTCGTCA | |
| | C_{H} | GCCAAAACAACAGCCCCATCGGTCTATCCACTGGCCCCTG | 86 |
| | | TGTGTGGAGATACAACTGGCTCCTCGGTGACTCTAGGATG | |
| | | CCTGGTCAAGGGTTATTTCCCTGAGCCAGTGACCTTGACC | |
| | | TGGAACTCTGGATCCCTGTCCAGTGGTGTGCACACCTTCC | |
| | | CAGCTGTCCTGCAGTCTGACCTCTACACCCTCAGCAGCTC | |
| | | AGTGACTGTAACCTCGAGCACCTGGCCCAGCCAGTCCATC | |
| | | ACCTGCAATGTGGCCCACCCGGCAAGCAGCACCAAGGTGG | |
| | | ACAAGAAAATTGAGCCCAGAGGGCCCACAATCAAGCCCTG | |
| | | TCCTCCATGCAAATGCCCAGCACCTAACCTCTTGGGTGGA | |
| | | CCATCCGTCTTCATCTTCCCTCCAAAGATCAAGGATGTAC | |
| | | TCATGATCTCCCTGAGCCCCATAGTCACATGTGTGGTGGT | |
| | | GGATGTGAGCGAGGATGACCCAGATGTCCAGATCAGCTGG | |
| | | TTTGTGAACAACGTGGAAGTACACACAGCTCAGACACAAA | |
| | | CCCATAGAGAGGATTACAACAGTACTCTCCGGGTGGTCAG | |
| | | TGCCCTCCCCATCCAGCACCAGGACTGGATGAGTGGCAAG | |
| | | GAGTTCAAATGCAAGGTCAACAACAAAGACCTCCCAGCGC | |
| | | CCATCGAGAGAACCATCTCAAAACCCAAAGGGTCAGTAAG | |
| | | AGCTCCACAGGTATATGTCTTGCCTCCACCAGAAGAAGAG | |
| | | ATGACTAAGAAACAGGTCACTCTGACCTGCATGGTCACAG | |
| | | ACTTCATGCCTGAAGACATTTACGTGGAGTGGACCAACAA | |
| | | CGGGAAAACAGAGCTAAACTACAAGAACACTGAACCAGTC | |
| | | CTGGACTCTGATGGTTCTTACTTCATGTACAGCAAGCTGA | |
| | | GAGTGGAAAAGAAGAACTGGGTGGAAAGAAATAGCTACTC | |
| | | CTGTTCAGTGGTCCACGAGGGTCTGCACAATCACCACACG | |
| | | ACTAAGAGCTTCTCCCGGACTCCGGGTAAA | |
| | HC | ATGGACTCCAGGCTCAATTTAGTTTTCCTTGTCCTTATTT | 87 |
| | signal | TAAAAGGTGTCCAATGT | |
| | V_{L} | GACATTGTGATGACCCAGTCTCAAAAATTCTTGTCCACAT | 59 |
| | | CAGTGGGAGACAGGGTCACCATCACCTGCAAGGCCAGTCA | |
| | | GAATGTGGGAACTGCTGTTGCCTGGTATCAACAGAAACCA | |
| | | GGGCAATCTCCTGAACTTCTGATTTACTCGGCATCCAATC | |
| | | GGCACACTGGAGTCCCTGCTCGCTTCACAGGCAGTGGATC | |
| | | TGGGACAGATTTCACTCTCACCATCACCAATATGCAGTCT | |
| | | GAAGACCTGGCAGATTATTTCTGCCAGCAATATAGTAGCT | |
| | | CGCCGACATTCGGTGGAGGCACCAAGTTGGAATCCAAA | |
| | C_{L} | CGGGCTGATGCTGCACCAACTGTATCCATCTTCCCACCAT | 88 |
| | | CCAGTGAGCAGTTAACATCTGGAGGTGCCTCAGTCGTGTG | |
| | | CTTCTTGAACAACTTCTACCCCAAAGACATCAATGTCAAG | |
| | | TGGAAGATTGATGGCAGTGAACGACAAAATGGCGTCCTGA | |
| | | ACAGTTGGACTGATCAGGACAGCAAAGACAGCACCTACAG | |
| | | CATGAGCAGCACCCTCACGTTGACCAAGGACGAGTATGAA | |
| | | CGACATAACAGCTATACCTGTGAGGCCACTCACAAGACAT | |
| | | CAACTTCACCCATTGTCAAGAGCTTCAACAGGAATGAGTG | |
| | | T | |
| | LC | ATGGAGTCTCATACTCAGGCCTTTGTATTCGCGTTTCTCT | 89 |
| | signal | GGTTGTCTGGTGTTGATGGA | |
| cAb4854 | V_{H} | GATGTGCAGCTGGTGGAGTCTGGGGGAGGCTTAGTGCAGC | 60 |
| 10F01B02 | | CTGGAGGGTCCCGGAAACTCTCCTGTGCAGCCTCTGGATT | |
| | | CCCTTTCAGTAGCTTTGGAATGCACTGGGTTCGTCAGGCT | |
| | | CCAGACAAGGGACTGGAGTGGGTCGCATACATTAGTAGTG | |
| | | CCAGTAATACCATCTACTATGCTGACACAGTGAAGGGCCG | |
| | | ATTCACCGTCTCCAGAGACAATCCCAAGAACACCCTGTTC | |
| | | CTGCAAATGACCAGTCTAAGGTCTGAGGACACGGCCATTT | |
| | | ATTACTGTACAAAGCGAAATTGGGACCTCTATGCTATGGA | |
| | | CTACTGGGGTCAAGGAACCTCAGTCACCGTCTCCTCA | |
| | C_{H} | GCCAAAACAACAGCCCCATCGGTCTATCCACTGGCCCCTG | 90 |
| | | TGTGTGGAGATACAACTGGCTCCTCGGTGACTCTAGGATG | |
| | | CCTGGTCAAGGGTTATTTCCCTGAGCCAGTGACCTTGACC | |
| | | TGGAACTCTGGATCCCTGTCCAGTGGTGTGCACACCTTCC | |
| | | CAGCTGTCCTGCAGTCTGACCTCTACACCCTCAGCAGCTC | |
| | | AGTGACTGTAACCTCGAGCACCTGGCCCAGCCAGTCCATC | |
| | | ACCTGCAATGTGGCCCACCCGGCAAGCAGCACCAAGGTGG | |
| | | ACAAGAAAATTGAGCCCAGAGGGCCCACAATCAAGCCCTG | |
| | | TCCTCCATGCAAATGCCCAGCACCTAACCTCTTGGGTGGA | |
| | | CCATCCGTCTTCATCTTCCCTCCAAAGATCAAGGATGTAC | |
| | | TCATGATCTCCCTGAGCCCCATAGTCACATGTGTGGTGGT | |
| | | GGATGTGAGCGAGGATGACCCAGATGTCCAGATCAGCTGG | |
| | | TTTGTGAACAACGTGGAAGTACACACAGCTCAGACACAAA | |
| | | CCCATAGAGAGGATTACAACAGTACTCTCCGGGTGGTCAG | |
| | | TGCCCTCCCCATCCAGCACCAGGACTGGATGAGTGGCAAG | |
| | | GAGTTCAAATGCAAGGTCAACAACAAAGACCTCCCAGCGC | |
| | | CCATCGAGAGAACCATCTCAAAACCCAAAGGGTCAGTAAG | |
| | | AGCTCCACAGGTATATGTCTTGCCTCCACCAGAAGAAGAG | |
| | | ATGACTAAGAAACAGGTCACTCTGACCTGCATGGTCACAG | |
| | | ACTTCATGCCTGAAGACATTTACGTGGAGTGGACCAACAA | |
| | | CGGGAAAACAGAGCTAAACTACAAGAACACTGAACCAGTC | |
| | | CTGGACTCTGATGGTTCTTACTTCATGTACAGCAAGCTGA | |
| | | GAGTGGAAAAGAAGAACTGGGTGGAAAGAAATAGCTACTC | |
| | | CTGTTCAGTGGTCCACGAGGGTCTGCACAATCACCACACG | |
| | | ACTAAGAGCTTCTCCCGGACTCCGGGTAAA | |
| | HC | ATGGACTCCAGGCTCAATTTAGTTTTCCTTGTCCTTATTT | 91 |
| | Signal | TAAAAGGTGTCCAGTGT | |
| | V_{L} | GACATTGTGATGACCCAGTCTCAAAAATTCATGTCCACAT | 61 |
| | | CAGTGGGAGACAGGGTCAGCATCACCTGCAAGGCCAATCA | |
| | | GAATGTGGGTACTGCTGTAGCCTGGTATCAACAGAAACCA | |
| | | GGACAATCTCCTAAACTACTGATTTATTCGACATCCAATC | |
| | | GGGACACTGGAGTCCCTGACCGCTTCACAGGCAGTGGATC | |
| | | TGGGACAGATTTCACTCTCACCATCAACTATATTCAATCT | |
| | | GAAGACCTGGCAGATTATTTCTGCCAGCAATATAGAAATT | |
| | | ATCCGACGTTCGGTGGAGGCACCAAGCTGGAAATCAAA | |
| | C_{L} | CGGGCTGATGCTGCACCAACTGTATCCATCTTCCCACCAT | 92 |
| | | CCAGTGAGCAGTTAACATCTGGAGGTGCCTCAGTCGTGTG | |
| | | CTTCTTGAACAACTTCTACCCCAAAGACATCAATGTCAAG | |
| | | TGGAAGATTGATGGCAGTGAACGACAAAATGGCGTCCTGA | |
| | | ACAGTTGGACTGATCAGGACAGCAAAGACAGCACCTACAG | |
| | | CATGAGCAGCACCCTCACGTTGACCAAGGACGAGTATGAA | |
| | | CGACATAACAGCTATACCTGTGAGGCCACTCACAAGACAT | |
| | | CAACTTCACCCATTGTCAAGAGCTTCAACAGGAATGAGTG | |
| | | T | |
| | LC | ATGGAGTCTCGTACTCAGGCCTTTGTAATCGCGTTTCTCT | 93 |
| | signal | GGATGTCTGGTATTGATGGA | |
| cAb4855 | V_{H} | CAGGTTACTCTGAAAGAGTCTGGCCCTGGGATATTGCAGC | 62 |
| 01F11A01 | | CCTCCCAGACCCTCAGTCTGACTTGTTCTTTCTCTGGGTT | |
| | | TTCACTGAGCACTTCTGGTATGGGTGTAGGCTGGATTCGT | |
| | | CAGCCTTCAGGGAAGGGTCTGGAGTGGCTGGCACACATTT | |
| | | GGTGGAATGATGAGAAATACTATAACGCAGCCCTGAAGAG | |
| | | CCGGCTCACAATCTCCAAGGATACCTCCAAAAACCAGGTT | |
| | | TTCCTCAAGATCGCCAGTGTGGACGCTGCAGATACTGCCA | |
| | | CATACTACTGTGCTCGCATCACTGGTGCCTGGTTTCCTTA | |
| | | TTGGGGCCAAGGGACTCTGGTCACTGTCTCTGCA | |
| | C_{H} | GCCAAAACAACAGCCCCATCGGTCTATCCACTGGCCCCTG | 94 |
| | | TGTGTGGAGATACAACTGGCTCCTCGGTGACTCTAGGATG | |
| | | CCTGGTCAAGGGTTATTTCCCTGAGCCAGTGACCTTGACC | |
| | | TGGAACTCTGGATCCCTGTCCAGTGGTGTGCACACCTTCC | |
| | | CAGCTGTCCTGCAGTCTGACCTCTACACCCTCAGCAGCTC | |
| | | AGTGACTGTAACCTCGAGCACCTGGCCCAGCCAGTCCATC | |
| | | ACCTGCAATGTGGCCCACCCGGCAAGCAGCACCAAGGTGG | |
| | | ACAAGAAAATTGAGCCCAGAGGGCCCACAATCAAGCCCTG | |
| | | TCCTCCATGCAAATGCCCAGCACCTAACCTCTTGGGTGGA | |
| | | CCATCCGTCTTCATCTTCCCTCCAAAGATCAAGGATGTAC | |
| | | TCATGATCTCCCTGAGTCCCATGGTCACATGTGTGGTGGT | |
| | | GGATGTGAGCGAGGATGACCCAGATGTCCAGATCAGCTGG | |
| | | TTCGTGAACAACGTGGAAGTACTCACAGCTCAGACACAAA | |
| | | CCCATAGAGAGGATTACAACAGTACTCTCCGGGTGGTCAG | |
| | | TGCCCTCCCCATCCAGCACCAGGACTGGATGAGTGGCAAG | |
| | | GAGTTCAAATGCAAGGTCAACAACAAAGACCTCCCAGCAC | |
| | | CCATCGAGAGAACCATCTCAAAACCCAAAGGGTCAGTAAG | |
| | | AGCTCCACAGGTATATGTCTTGCCTCCACCAGAAGAAGAG | |
| | | ATGACTAAGAAACAGGTCACTCTGACCTGCATGGTCACAG | |
| | | ACTTCATGCCTGAAGACATTTACGTGGAGTGGACCAACAA | |
| | | CGGGAAAACAGAGCTAAACTACAAGAACACTGAACCAGTC | |
| | | CTGGACTCTGATGGTTCTTACTTCATGTACAGCAAGCTGA | |
| | | GAGTGGAAAAGAAGAACTGGGTGGAAAGAAATAGCTACTC | |
| | | CTGTTCAGTGGTCCACGAGGGTCTGCACAATCACCACACG | |
| | | ACTAAGAGCTTCTCCCGGACTCCGGGTAAA | |
| | HC | ATGGGCAGACTTACTTCTTCATTCTTGCTACTGATTGTCC | 95 |
| | Signal | CTGCATATGTCCTGTCC | |
| | V_{L} | GACATTGTGATGACTCAGTCTCCAGCCACCCTGTCTGTGA | 63 |
| | | CTCCAGGAGATAGAGTCTCTCTTTCCTGCAGGGCCAGTCA | |
| | | GCATATTAGCGAATACTTACACTGGTATCAACAAAAATCA | |
| | | CACGAGTCTCCAAGGCTTCTCATCAAATATGGTTCCCAAT | |
| | | CCATCTCTGGGATCCCCTCCAGGTTCAGTGGCAGTGGATC | |
| | | AGGGTCAGATTTCACTCTCAATATCAACAGTGTGGAACCT | |
| | | GAAGATGTTGGAGTGTATTACTGTCAAAATGGTCACAGCT | |
| | | TTCCGTACACGTTCGGAGGGGGGACCAAGCTGGAAATAAA | |
| | | A | |
| | C_{L} | CGGGCTGATGCTGCACCAACTGTATCCATCTTCCCACCAT | 96 |
| | | CCAGTGAGCAGTTAACATCTGGAGGTGCCTCAGTCGTGTG | |
| | | CTTCTTGAACAACTTCTACCCCAAAGACATCAATGTCAAG | |
| | | TGGAAGATTGATGGCAGTGAACGACAAAATGGCGTCCTGA | |
| | | ACAGTTGGACTGATCAGGACAGCAAAGACAGCACCTACAG | |
| | | CATGAGCAGCACCCTCACGTTGACCAAGGACGAGTATGAA | |
| | | CGACATAACAGCTATACCTGTGAGGCCACTCACAAGACAT | |
| | | CAACTTCACCCATTGTCAAGAGCTTCAACAGGAATGAGTG | |
| | | T | |
| | LC | ATGGTGTCCACTTCTCAGCTCCTTGGACTTTTGCTTTTCT | 97 |
| | signal | GGACTTCAGCCTCCAGATGT | |
| cAb5775 | V_{H} | GAGGTCCAGCTGCAACAGTCTGGACCTGAGCTGGTGAAGC | 64 |
| 12D02 | | CTGGAGCCTCAATGAAGATATCTTGCAAGGCTTCTGGTTA | |
| | | CTCATTCACTGCCTACACCATGAACTGGGTGAAGCAGAGC | |
| | | CATGGAGAGAACCTTGAGTGGATTGGACTTATTAATCCTT | |
| | | ATAATGGTGGTACTACCTACAACCAGAAGTTCAAGGGCAA | |
| | | GGCCACATTAACTGTAGACAAGTCATCCAGCACAGCCTAC | |
| | | ATGGAGCTCCTCAGTCTGACATCTGAGGACTCTGCAGTCT | |
| | | ATTACTGTGCATCATATGATTACGGCTATGCTATGGACTA | |
| | | CTGGGGTCAAGGAACCTCAGTCACCGTCTCCTCA | |
| | C_{H} | GCCAAAACGACACCCCCATCTGTCTATCCACTGGCCCCTG | 98 |
| | | GATCTGCTGCCCAAACTAACTCCATGGTGACCCTGGGATG | |
| | | CCTGGTCAAGGGCTATTTCCCTGAGCCAGTGACAGTGACC | |
| | | TGGAACTCTGGATCCCTGTCCAGCGGTGTGCACACCTTCC | |
| | | CAGCTGTCCTGCAGTCTGACCTCTACACTCTGAGCAGCTC | |
| | | AGTGACTGTCCCCTCCAGCACCTGGCCCAGCGAGACCGTC | |
| | | ACCTGCAACGTTGCCCACCCGGCCAGCAGCACCAAGGTGG | |
| | | ACAAGAAAATTGTGCCCAGGGATTGTGGTTGTAAGCCTTG | |
| | | CATATGTACAGTCCCAGAAGTATCATCTGTCTTCATCTTC | |
| | | CCCCCAAAGCCCAAGGATGTGCTCACCATTACTCTGACTC | |
| | | CTAAGGTCACGTGTGTTGTGGTAGACATCAGCAAGGATGA | |
| | | TCCCGAGGTCCAGTTCAGCTGGTTTGTAGATGATGTGGAG | |
| | | GTGCACACAGCTCAGACGCAACCCCGGGAGGAGCAGTTCA | |
| | | ACAGCACTTTCCGCTCAGTCAGTGAACTTCCCATCATGCA | |
| | | CCAGGACTGGCTCAATGGCAAGGAGTTCAAATGCAGGGTC | |
| | | AACAGTGCAGCTTTCCCTGCCCCCATCGAGAAAACCATCT | |
| | | CCAAAACCAAAGGCAGACCGAAGGCTCCACAGGTGTACAC | |
| | | CATTCCACCTCCCAAGGAGCAGATGGCCAAGGATAAAGTC | |
| | | AGTCTGACCTGCATGATAACAGACTTCTTCCCTGAAGACA | |
| | | TTACTGTGGAGTGGCAGTGGAATGGGCAGCCAGCGGAGAA | |
| | | CTACAAGAACACTCAGCCCATCATGGACACAGATGGCTCT | |
| | | TACTTCGTCTACAGCAAGCTCAATGTGCAGAAGAGCAACT | |
| | | GGGAGGCAGGAAATACTTTCACCTGCTCTGTGTTACATGA | |
| | | GGGCCTGCACAACCACCATACTGAGAAGAGCCTCTCCCAC | |
| | | TCTCCTGGTAAA | |
| | HC | ATGGGATGGAGCTGGATCTTTCTCTTCCTCCTGTCAGGAA | 99 |
| | Signal | CTGCAGGTGTCCACTCT | |
| | V_{L} | GATGTTTTGATGACCCAAACTCCACTCTCCCTGCCTGTCA | 65 |
| | | GTCTTGGAGATCAAGCCTCCATCTCTTGCAGATCTAGTCA | |
| | | GAGCATTGTACATAGAAATGGAAACACCTATCTAGAATGG | |
| | | TACCTGCAGAAACCAGGCCAGTCTCCAAAGCTCCTGATCT | |
| | | ACAAAGTTTCCAACCGATTTTCTGGGGTCCCAGACAGGTT | |
| | | CAGTGGCAGTGGATCAGGGACAGATTTCACACTCAAGATC | |
| | | AGCAGAGTGGAGGCTGAGGATCTGGGAGTTTATTACTGCT | |
| | | TTCAAGGTTCACATGTTCCGTGGACGTTCGGTGGAGGCAC | |
| | | CAAGCTGGAAATCAAA | |
| | C_{L} | CGGGCTGATGCTGCACCAACTGTATCCATCTTCCCACCAT | 100 |
| | | CCAGTGAGCAGTTAACATCTGGAGGTGCCTCAGTCGTGTG | |
| | | CTTCTTGAACAACTTCTACCCCAAAGACATCAATGTCAAG | |
| | | TGGAAGATTGATGGCAGTGAACGACAAAATGGCGTCCTGA | |
| | | ACAGTTGGACTGATCAGGACAGCAAAGACAGCACCTACAG | |
| | | CATGAGCAGCACCCTCACGTTGACCAAGGACGAGTATGAA | |
| | | CGACATAACAGCTATACCTGTGAGGCCACTCACAAGACAT | |
| | | CAACTTCACCCATTGTCAAGAGCTTCAACAGGAATGAGTG | |
| | | T | |
| | LC | ATGAAGTTGCCTGTTAGGCTGTTGGTGCTGATGTTCTGGA | 101 |
| | signal | TTCCTGCTTCCAGTAGT | |
| cAb5776 | V_{H} | GAGGTCCAGCTGCAACAGTCTGGACCTGAGCTGGTGAAGC | 66 |
| 12A12 | | CTGGAGTTTCAATGAAGATATCCTGCAAGGCTTCTGGTTA | |
| | | CTCATTCACTGCCTACACTATGAATTGGGTGAAGCAGAGT | |
| | | CATGGAGAGAACCTTGAGTGGATTGGACTTATTAATCCTT | |
| | | ATAATGGTGGTACTACCTACAACCAGAAGTTCAAGGGCAA | |
| | | GGCCACATTAACTGTAGACAAGTCATCCAGCACAGCCTAC | |
| | | ATGGAGCTCCTCAGTCTGACATCTGAGGACTCTGCAGTCT | |
| | | ATTACTGTGCATCATATGATTACGGCTATGCTATGGACTA | |
| | | CTGGGGTCAAGGAACCTCAGTCACCGTCTCCTCA | |
| | C_{H} | GCCAAAACGACACCCCCATCTGTCTATCCACTGGCCCCTG | 102 |
| | | GATCTGCTGCCCAAACTAACTCCATGGTGACCCTGGGATG | |
| | | CCTGGTCAAGGGCTATTTCCCTGAGCCAGTGACAGTGACC | |
| | | TGGAACTCTGGATCCCTGTCCAGCGGTGTGCACACCTTCC | |
| | | CAGCTGTCCTGCAGTCTGACCTCTACACTCTGAGCAGCTC | |
| | | AGTGACTGTCCCCTCCAGCACCTGGCCCAGCGAGACCGTC | |
| | | ACCTGCAACGTTGCCCACCCGGCCAGCAGCACCAAGGTGG | |
| | | ACAAGAAAATTGTGCCCAGGGATTGTGGTTGTAAGCCTTG | |
| | | CATATGTACAGTCCCAGAAGTATCATCTGTCTTCATCTTC | |
| | | CCCCCAAAGCCCAAGGATGTGCTCACCATTACTCTGACTC | |
| | | CTAAGGTCACGTGTGTTGTGGTAGACATCAGCAAGGATGA | |
| | | TCCCGAGGTCCAGTTCAGCTGGTTTGTAGATGATGTGGAG | |
| | | GTGCACACAGCTCAGACGCAACCCCGGGAGGAGCAGTTCA | |
| | | ACAGCACTTTCCGCTCAGTCAGTGAACTTCCCATCATGCA | |
| | | CCAGGACTGGCTCAATGGCAAGGAGTTCAAATGCAGGGTC | |
| | | AACAGTGCAGCTTTCCCTGCCCCCATCGAGAAAACCATCT | |
| | | CCAAAACCAAAGGCAGACCGAAGGCTCCACAGGTGTACAC | |
| | | CATTCCACCTCCCAAGGAGCAGATGGCCAAGGATAAAGTC | |
| | | AGTCTGACCTGCATGATAACAGACTTCTTCCCTGAAGACA | |
| | | TTACTGTGGAGTGGCAGTGGAATGGGCAGCCAGCGGAGAA | |
| | | CTACAAGAACACTCAGCCCATCATGGACACAGATGGCTCT | |
| | | TACTTCGTCTACAGCAAGCTCAATGTGCAGAAGAGCAACT | |
| | | GGGAGGCAGGAAATACTTTCACCTGCTCTGTGTTACATGA | |
| | | GGGCCTGCACAACCACCATACTGAGAAGAGCCTCTCCCAC | |
| | | TCTCCTGGTAAA | |
| | HC | ATGGGATGGAGCTGGATCTTTCTCTTCCTCCTGTCAGGAA | 103 |
| | Signal | CTGCAGGTGTCCACTCT | |
| | V_{L} | GATGTTTTGATGACCCAAACTCCACTCTCCCTGCCTGTCA | 67 |
| | | GTCTTGGAGATCAAGCCTCCATCTCTTGCAGATCTAGTCA | |
| | | GAGCATTGTACATCGTAGTGGAAACACCTATTTAGAATGG | |
| | | TACCTGCAGAAACCAGGCCAGTCTCCAAAGCTCCTGATCT | |
| | | ACAAAGTTTCCAACCGATTTTCTGGGGTCCCAGACAGGTT | |
| | | CAGTGGCAGTGGATCAGGGACAGATTTCACACTCAAGATC | |
| | | AGCAGAGTGGAGGCTGAGGATCTGGGAGTTTATTACTGCT | |
| | | TTCAAGGTTCACATGTTCCGTGGACGTTCGGTGGAGGCAC | |
| | | CAAGTTGGAAATCAAA | |
| | C_{L} | CGGGCTGATGCTGCACCAACTGTATCCATCTTCCCACCAT | 104 |
| | | CCAGTGAGCAGTTAACATCTGGAGGTGCCTCAGTCGTGTG | |
| | | CTTCTTGAACAACTTCTACCCCAAAGACATCAATGTCAAG | |
| | | TGGAAGATTGATGGCAGTGAACGACAAAATGGCGTCCTGA | |
| | | ACAGTTGGACTGATCAGGACAGCAAAGACAGCACCTACAG | |
| | | CATGAGCAGCACCCTCACGTTGACCAAGGACGAGTATGAA | |
| | | CGACATAACAGCTATACCTGTGAGGCCACTCACAAGACAT | |
| | | CAACTTCACCCATTGTCAAGAGCTTCAACAGGAATGAGTG | |
| | | T | |
| | LC | ATGAAGTTGCCTGTTAGGCTGTTGGTGCTGATGTTCTGGA | 105 |
| | signal | TTCCTGCTTCCAGCAGT | |
| cAb5777 | V_{H} | GAGGTTCAACTGCAGCAGTCTGGGGCAGAGCTTGTGAAGC | 68 |
| 12D08 | | CAGGGGCCTCAGTCAAGTTGTCCTGCACAGCTTCTGGCTT | |
| | | CAACATTAAAGACACCTATATGCACTGGGTGAAACAGAGG | |
| | | CCTGAGCAGGGCCTGATTTGGATTGGAAACATTGATCCTG | |
| | | CGAATGGTCATACTAAATATGCCCCGAAGTTCCAGGGCAA | |
| | | GGCCACTATCACAGCAGACACATCCTCCAACACAGCCTAC | |
| | | CTGCAGCTCAGCAGCCTGACATCTGAGGACACTGCCGTCT | |
| | | ATTACTGTGCTAGATCTAATGATTACGACGTCGACTTTGA | |
| | | CTACTGGGGCCAAGGCACCACTCTCACAGTCTCCTCA | |
| | C_{H} | GCCAAAACAACAGCCCCATCGGTCTATCCACTGGCCCCTG | 106 |
| | | TGTGTGGAGATACAACTGGCTCCTCGGTGACTCTAGGATG | |
| | | CCTGGTCAAGGGTTATTTCCCTGAGCCAGTGACCTTGACC | |
| | | TGGAACTCTGGATCCCTGTCCAGTGGTGTGCACACCTTCC | |
| | | CAGCTGTCCTGCAGTCTGACCTCTACACCCTCAGCAGCTC | |
| | | AGTGACTGTAACCTCGAGCACCTGGCCCAGCCAGTCCATC | |
| | | ACCTGCAATGTGGCCCACCCGGCAAGCAGCACCAAGGTGG | |
| | | ACAAGAAAATTGAGCCCAGAGGGCCCACAATCAAGCCCTG | |
| | | TCCTCCATGCAAATGCCCAGCACCTAACCTCTTGGGTGGA | |
| | | CCATCCGTCTTCATCTTCCCTCCAAAGATCAAGGATGTAC | |
| | | TCATGATCTCCCTGAGCCCCATAGTCACATGTGTGGTGGT | |
| | | GGATGTGAGCGAGGATGACCCAGATGTCCAGATCAGCTGG | |
| | | TTTGTGAACAACGTGGAAGTACACACAGCTCAGACACAAA | |
| | | CCCATAGAGAGGATTACAACAGTACTCTCCGGGTGGTCAG | |
| | | TGCCCTCCCCATCCAGCACCAGGACTGGATGAGTGGCAAG | |
| | | GAGTTCAAATGCAAGGTCAACAACAAAGACCTCCCAGCGC | |
| | | CCATCGAGAGAACCATCTCAAAACCCAAAGGGTCAGTAAG | |
| | | AGCTCCACAGGTATATGTCTTGCCTCCACCAGAAGAAGAG | |
| | | ATGACTAAGAAACAGGTCACTCTGACCTGCATGGTCACAG | |
| | | ACTTCATGCCTGAAGACATTTACGTGGAGTGGACCAACAA | |
| | | CGGGAAAACAGAGCTAAACTACAAGAACACTGAACCAGTC | |
| | | CTGGACTCTGATGGTTCTTACTTCATGTACAGCAAGCTGA | |
| | | GAGTGGAAAAGAAGAACTGGGTGGAAAGAAATAGCTACTC | |
| | | CTGTTCAGTGGTCCACGAGGGTCTGCACAATCACCACACG | |
| | | ACTAAGAGCTTCTCCCGGACTCCGGGTAAA | |
| | HC | ATGAAATGCAGCTGGGTTATCTTCTTCCTGATGGCAGTGG | 107 |
| | Signal | TTACAGGGGTCAATTCA | |
| | V_{L} | GACATTGTGATGTCACAGTCTCCATCCTCCCTGGCTGTGT | 69 |
| | | CAGCAGGAGAGAAGGTCACTATGAGCTGCAAATCCAGTCA | |
| | | GAGTCTGCTCAACAGTAGAACCCGAAAGAACTACTTGGCT | |
| | | TGGTACCAGCAGAAACCAGGGCAGTCTCCTAAACTGCTGA | |
| | | TCTACTGGGCATCCACTAGGGAATCTGGGGTCCCTGATCG | |
| | | CTTCACAGGCAGTGGATCTGGGACAGATTTCAGTCTCACC | |
| | | ATCAGCAGTGTGCAGGCTGAAGACCTGGCAGTTTATTACT | |
| | | GCAAGCAATCTTATAATCTATACACGTTCGGAGGGGGGAC | |
| | | CAAGCTGGAAATAAAA | |
| | C_{L} | CGGGCTGATGCTGCACCAACTGTATCCATCTTCCCACCAT | 108 |
| | | CCAGTGAGCAGTTAACATCTGGAGGTGCCTCAGTCGTGTG | |
| | | CTTCTTGAACAACTTCTACCCCAAAGACATCAATGTCAAG | |
| | | TGGAAGATTGATGGCAGTGAACGACAAAATGGCGTCCTGA | |
| | | ACAGTTGGACTGATCAGGACAGCAAAGACAGCACCTACAG | |
| | | CATGAGCAGCACCCTCACGTTGACCAAGGACGAGTATGAA | |
| | | CGACATAACAGCTATACCTGTGAGGCCACTCACAAGACAT | |
| | | CAACTTCACCCATTGTCAAGAGCTTCAACAGGAATGAGTG | |
| | | T | |
| | LC | ATGGATTCACGGGCCCAGGTTCTTATATTGCTGCTGCAAT | 109 |
| | signal | GGGTATCTGGTACCTGTGGG | |
| cAb5778 | V_{H} | GAGGTCCACCTGCAACAGTCTGGACCTGAGCTGGTGAAGC | 70 |
| 13G04 | | CTGGAGCTTCAATGAAGATATCCTGCAAGGCTTCTGGTTA | |
| | | CTCCTTCACTGCCTACACCATGAACTGGGTGAAACAGAGC | |
| | | CATGGAAAGAACCTTGAGTGGATTGGACTTATTAATCCTT | |
| | | ACAATGGTGGTACTACCTACAACCAGAAATTCAAGGGCAA | |
| | | GGCCACATTAACTGTAGACAAGTCATCCAGCACAGCCTAC | |
| | | ATGGAGCTCCTCAGTCTGACATCTGAGGACTCTGCAGTCT | |
| | | ATTACTGTGCTTCTTACTACGGTGGTAGGGGCTGGTACTT | |
| | | CGATGTCTGGGGCGCAGGGACCTCGGTCACCGTCTCCTCA | |
| | | GCCAAAACGACACCCCCATCTGTCTATCCACTGGCCCCTG | |
| | | GATCTGCTGCCCAAACTAACTCCATGGTGACCCTGGGATG | |
| | | CCTGGTCAAGGGCTATTTCCCTGAGCCAGTGACAGTGACC | |
| | | TGGAACTCTGGATCCCTGTCCAGCGGTGTGCACACCTTCC | |
| | | CAGCTGTCCTGCAGTCTGACCTCTACACTCTGAGCAGCTC | |
| | | AGTGACTGTCCCCTCCAGCACCTGGCCCAGCGAGACCGTC | |
| | | ACCTGCAACGTTGCCCACCCGGCCAGCAGCACCAAGGTGG | |
| | | ACAAGAAAATTGTGCCCAGGGATTGTGGTTGTAAGCCTT | |
| | C_{H} | GCATATGTACAGTCCCAGAAGTATCATCTGTCTTCATCTT | 110 |
| | | CCCCCCAAAGCCCAAGGATGTGCTCACCATTACTCTGACT | |
| | | CCTAAGGTCACGTGTGTTGTGGTAGACATCAGCAAGGATG | |
| | | ATCCCGAGGTCCAGTTCAGCTGGTTTGTAGATGATGTGGA | |
| | | GGTGCACACAGCTCAGACGCAACCCCGGGAGGAGCAGTTC | |
| | | AACAGCACTTTCCGCTCAGTCAGTGAACTTCCCATCATGC | |
| | | ACCAGGACTGGCTCAATGGCAAGGAGTTCAAATGCAGGGT | |
| | | CAACAGTGCAGCTTTCCCTGCCCCCATCGAGAAAACCATC | |
| | | TCCAAAACCAAAGGCAGACCGAAGGCTCCACAGGTGTACA | |
| | | CCATTCCACCTCCCAAGGAGCAGATGGCCAAGGATAAAGT | |
| | | CAGTCTGACCTGCATGATAACAGACTTCTTCCCTGAAGAC | |
| | | ATTACTGTGGAGTGGCAGTGGAATGGGCAGCCAGCGGAGA | |
| | | ACTACAAGAACACTCAGCCCATCATGGACACAGATGGCTC | |
| | | TTACTTCGTCTACAGCAAGCTCAATGTGCAGAAGAGCAAC | |
| | | TGGGAGGCAGGAAATACTTTCACCTGCTCTGTGTTACATG | |
| | | AGGGCCTGCACAACCACCATACTGAGAAGAGCCTCTCCCA | |
| | | CTCTCCTGGTAAA | |
| | HC | ATGGGATGGAGCTGGATCTTTCTCTTCCTCCTGTCAGGAA | 111 |
| | Signal | CTGCAGGTGTCCACTCT | |
| | V_{L} | GACATTGTGCTGACACAGTCTCCTGCTTCCTTAGCTGTAT | 71 |
| | | CTCTGGGGCAGAGGGCCACCATCTCATGCAGGGCCAGCAA | |
| | | AAGTGTCAGTACATCTGGCTATAGTTATGTTCACTGGTAC | |
| | | CAACAGAAACCAGGACAGCCACCCAAACTCCTCATCTATC | |
| | | TTGCATCCAACCTAGAATCTGGGGTCCCTGCCAGGTTCAG | |
| | | TGGCAGTGGGTCTGGGACAGACTTCACCCTCAACATCCAT | |
| | | CCTGTGGAGGAGGAAGATGCTGCAACCTATTACTGTCAGA | |
| | | ATAGTAGGGAACTTCCGTACACGTTCGGAGGGGGGACCA | |
| | | AGCTGGAAATGAAA | |
| | C_{L} | CGGGCTGATGCTGCACCAACTGTATCCATCTTCCCACCAT | 112 |
| | | CCAGTGAGCAGTTAACATCTGGAGGTGCCTCAGTCGTGTG | |
| | | CTTCTTGAACAACTTCTACCCCAAAGACATCAATGTCAAG | |
| | | TGGAAGATTGATGGCAGTGAACGACAAAATGGCGTCCTGA | |
| | | ACAGTTGGACTGATCAGGACAGCAAAGACAGCACCTACAG | |
| | | CATGAGCAGCACCCTCACGTTGACCAAGGACGAGTATGAA | |
| | | CGACATAACAGCTATACCTGTGAGGCCACTCACAAGACAT | |
| | | CAACTTCACCCATTGTCAAGAGCTTCAACAGGAATGAGTG | |
| | | T | |
| | LC | ATGGAGACAGACACACTCCTGTTATGGGTACTGCTGCTCT | 113 |
| | signal | GGGTTCCAGGTTCCACTGGT | |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An antibody-based molecule that binds to human cell migration-inducing and hyaluronan-binding protein (CEMIP), said antibody-based molecule comprising a heavy chain variable region, wherein said heavy chain variable region comprises:
a complementarity-determining region 1 (CDR-H1) comprising an amino acid sequence of any one of SEQ ID NOs: 2-8 or a modified amino acid sequence of any one of SEQ ID NOs: 2-8, said modified sequence having at least 80% sequence identity to any one of SEQ ID NOs: 2-8;
a complementarity-determining region 2 (CDR-H2) comprising an amino acid sequence of any one of SEQ ID NOs: 9-15 or a modified amino acid sequence of any one of SEQ ID NOs: 9-15, said modified sequences having at least 80% sequence identity to any one of SEQ ID NOs: 9-15; and
a complementarity-determining region 3 (CDR-H3) comprising an amino acid sequence of any one of SEQ ID NOs: 16-22, or a modified amino acid sequence of any one of SEQ ID NO: 16-22, said modified sequence having at least 80% sequence identity to any one of SEQ ID NOs: 16-22.

2. The antibody-based molecule of claim 1, wherein said heavy chain variable region is selected from the group consisting of:
(i) a heavy chain variable region comprising the CDR-H1 of SEQ ID NO: 2, the CDR-H2 of SEQ ID NO: 9, and the CDR-H3 of SEQ ID NO: 16;
(ii) a heavy chain variable region comprising the CDR-H1 of SEQ ID NO: 3, the CDR-H2 of SEQ ID NO: 10, and the CDR-H3 of SEQ ID NO: 17;
(iii) a heavy chain variable region comprising the CDR-H1 of SEQ ID NO: 4, the CDR-H2 of SEQ ID NO: 11, and the CDR-H3 of SEQ ID NO: 18;
(iv) a heavy chain variable region comprising the CDR-H1 of SEQ ID NO: 5, the CDR-H2 of SEQ ID NO: 12, and the CDR-H3 of SEQ ID NO: 19;
(v) a heavy chain variable region comprising the CDR-H1 of SEQ ID NO: 6, the CDR-H2 of SEQ ID NO: 13, and the CDR-H3 of SEQ ID NO: 20;
(vi) a heavy chain variable region comprising the CDR-H1 of SEQ ID NO: 7, the CDR-H2 of SEQ ID NO: 14, and the CDR-H3 of SEQ ID NO: 21; and
(vii) a heavy chain variable region comprising the CDR-H1 of SEQ ID NO: 8, the CDR-H2 of SEQ ID NO: 15, and the CDR-H3 of SEQ ID NO: 22.

3. The antibody-based molecule of claim 1 or claim 2, wherein said heavy chain variable region of said antibody-based molecule further comprises human or humanized immunoglobulin heavy chain framework regions.

4. The antibody-based molecule of any one of claims 1-3, wherein said antibody-based molecule comprises:
(i) a heavy chain variable region comprising an amino acid sequence that is at least 80% identical to SEQ ID NO: 44;
(ii) a heavy chain variable region comprising an amino acid sequence that is at least 80% identical to SEQ ID NO: 46;
(iii) a heavy chain variable region comprising an amino acid sequence that is at least 80% identical to SEQ ID NO: 48;
(iv) a heavy chain variable region comprising an amino acid sequence that is at least 80% identical to SEQ ID NO: 50;
(v) a heavy chain variable region comprising an amino acid sequence that is at least 80% identical to SEQ ID NO: 52;
(vi) a heavy chain variable region comprising an amino acid sequence that is at least 80% identical to SEQ ID NO: 54; or
(vii) a heavy chain variable region comprising an amino acid sequence that is at least 80% identical to SEQ ID NO: 56.

5. The antibody-based molecule of any one of claims 1-4, wherein said antibody is a single domain antibody.

6. The antibody-based molecule of any one of claims 1-4, wherein said antibody-based molecule further comprises a light chain variable region, wherein said light chain variable region comprises:
a complementarity-determining region 1 (CDR-L1) having an amino acid sequence of any one of SEQ ID NOs: 23-29, or a modified amino acid sequence of any one of SEQ ID NO: 23-29, said modified sequence having at least 80% sequence identity to any one of SEQ ID NO: 23-29;
a complementarity-determining region 2 (CDR-L2) having an amino acid sequence of any one of SEQ ID NOs: 30-36, or a modified amino acid sequence of any one of SEQ ID NO: 30-36, said modified sequence having at least 80% sequence identity to any one of SEQ ID NO: 30-36; and
a complementarity-determining region 3 (CDR-L3) having an amino acid sequence of any one of SEQ ID NOs: 37-43, or a modified amino acid sequence of any one of SEQ ID NO: 37-43, said modified sequence having at least 80% sequence identity to any one of SEQ ID NO: 37-43.

7. The antibody-based molecule of claim 6, wherein said light chain variable region is selected from the group consisting of:
(i) a light chain variable region comprising the CDR-L1 of SEQ ID NO: 23, the CDR-L2 of SEQ ID NO: 30, and the CDR-L3 of SEQ ID NO: 37;
(ii) a light chain variable region comprising the CDR-L1 of SEQ ID NO: 24, the CDR-L2 of SEQ ID NO: 31, and the CDR-L3 of SEQ ID NO: 38;
(iii) a light chain variable region comprising the CDR-L1 of SEQ ID NO: 25, the CDR-L2 of SEQ ID NO: 32, and the CDR-L3 of SEQ ID NO: 39;
(iv) a light chain variable region comprising the CDR-L1 of SEQ ID NO: 26, the CDR-L2 of SEQ ID NO: 33, and the CDR-L3 of SEQ ID NO: 40;
(v) a light chain variable region comprising the CDR-L1 of SEQ ID NO: 27, the CDR-L2 of SEQ ID NO: 34, and the CDR-L3 of SEQ ID NO: 41;
(vi) a light chain variable region comprising the CDR-L1 of SEQ ID NO: 28, the CDR-L2 of SEQ ID NO: 35, and the CDR-L3 of SEQ ID NO: 42; or
(vii) a light chain variable region comprising the CDR-L1 of SEQ ID NO: 29, the CDR-L2 of SEQ ID NO: 36, and the CDR-L3 of SEQ ID NO: 43.

8. The antibody-based molecule of claim 7, wherein said light chain variable region of said antibody-based molecule further comprises human or humanized immunoglobulin light chain framework regions.

9. The antibody-based molecule of claim 7, wherein said antibody or binding fragment thereof comprises:
(i) a heavy chain variable region comprising the CDR-H1 of SEQ ID NO: 2, the CDR-H2 of SEQ ID NO: 9, and the CDR-H3 of SEQ ID NO: 16, and a light chain variable region comprising the CDR-L1 of SEQ ID NO: 23, the CDR-L2 of SEQ ID NO: 30, and the CDR-L3 of SEQ ID NO: 37;
(ii) a heavy chain variable region comprising the CDR-H1 of SEQ ID NO: 3, the CDR-H2 of SEQ ID NO: 10, and the CDR-H3 of SEQ ID NO: 17, and a light chain variable region comprising the CDR-L1 of SEQ ID NO: 24, the CDR-L2 of SEQ ID NO: 31, and the CDR-L3 of SEQ ID NO: 38;
(iii) a heavy chain variable region comprising the CDR-H1 of SEQ ID NO: 4, the CDR-H2 of SEQ ID NO: 11, and the CDR-H3 of SEQ ID NO: 18, and a light chain variable region comprising the CDR-L1 of SEQ ID NO: 25, the CDR-L2 of SEQ ID NO: 32, and the CDR-L3 of SEQ ID NO: 39;
(iv) a heavy chain variable region comprising the CDR-H1 of SEQ ID NO: 5, the CDR-H2 of SEQ ID NO: 12, and the CDR-H3 of SEQ ID NO: 19, and a light chain variable region comprising the CDR-L1 of SEQ ID NO: 26, the CDR-L2 of SEQ ID NO: 33, and the CDR-L3 of SEQ ID NO: 40;
(v) a heavy chain variable region comprising the CDR-H1 of SEQ ID NO: 6, the CDR-H2 of SEQ ID NO: 13, and the CDR-H3 of SEQ ID NO: 20, and a light chain variable region comprising the CDR-L1 of SEQ ID NO: 27, the CDR-L2 of SEQ ID NO: 34, and the CDR-L3 of SEQ ID NO: 41;
(vi) a heavy chain variable region comprising the CDR-H1 of SEQ ID NO: 7, the CDR-H2 of SEQ ID NO: 14, and the CDR-H3 of SEQ ID NO: 21, and a light chain variable region comprising the CDR-L1 of SEQ ID NO: 28, the CDR-L2 of SEQ ID NO: 35, and the CDR-L3 of SEQ ID NO: 42; or
(vii) a heavy chain variable region comprising the CDR-H1 of SEQ ID NO: 8, the CDR-H2 of SEQ ID NO: 15, and the CDR-H3 of SEQ ID NO: 22, and a light chain variable region comprising the CDR-L1 of SEQ ID NO: 29, the CDR-L2 of SEQ ID NO: 36, and the CDR-L3 of SEQ ID NO: 43.

10. The antibody-based molecule of claim 9, wherein said antibody-based molecule comprises:
(i) a heavy chain variable region comprising an amino acid sequence that is at least 80% identical to SEQ ID NO: 44 and a light chain variable region comprising an amino acid sequence that is at least 80% identical to SEQ ID NO: 45;
(ii) a heavy chain variable region comprising an amino acid sequence that is at least 80% identical to SEQ ID NO: 46 and a light chain variable region comprising an amino acid sequence that is at least 80% identical to SEQ ID NO: 47;
(iii) a heavy chain variable region comprising an amino acid sequence that is at least 80% identical to SEQ ID NO: 48 and a light chain variable region comprising an amino acid sequence that is at least 80% identical to SEQ ID NO: 49;
(iv) a heavy chain variable region comprising an amino acid sequence that is at least 80% identical to SEQ ID NO: 50 and a light chain variable region comprising an amino acid sequence that is at least 80% identical to SEQ ID NO: 51;
(v) a heavy chain variable region comprising an amino acid sequence that is at least 80% identical to SEQ ID NO: 52 and a light chain variable region comprising an amino acid sequence that is at least 80% identical to SEQ ID NO: 53;
(vi) a heavy chain variable region comprising an amino acid sequence that is at least 80% identical to SEQ ID NO: 54 and a light chain variable region comprising an amino acid sequence that is at least 80% identical to SEQ ID NO: 55; or
(vii) a heavy chain variable region comprising an amino acid sequence that is at least 80% identical to SEQ ID NO: 56 and a light chain variable region comprising an amino acid sequence that is at least 80% identical to SEQ ID NO: 57.

11. The antibody-based molecule of any one of claims 1-10, wherein said antibody-based molecule is a chimeric antibody or binding fragment thereof.

12. The antibody-based molecule of any one of claims 1-10, wherein said antibody-based molecule is a humanized antibody or binding fragment thereof.

13. The antibody-based molecule of any one of claims 1-12, wherein said antibody-based molecule is a monoclonal antibody or binding fragment thereof.

14. The antibody-based molecule of any one of claims 1-12, wherein said antibody-based molecule is a full-length antibody, an epitope-binding fragment of an antibody, or an antibody derivative.

15. The antibody-based molecule of claim 14, wherein said antibody-based molecule is an epitope binding fragment selected from a F(ab) fragment, a F(ab′) fragment, and F(ab′)₂ fragment.

16. The antibody-based molecule of claim 14, where said antibody-based molecule is an antibody derivative selected from the group consisting of a scFv, a minibody, a diabody, a triabody, and a tetrabody.

17. An isolated polynucleotide encoding the antibody-based molecule of any one of claims 1-16.

18. A vector comprising the isolated polynucleotide of claim 17.

19. A host cell comprising the vector of claim 18.

20. A pharmaceutical composition comprising:
the antibody-based molecule of any one of claims 1-16, the polynucleotide of claim 17, or the vector of claim 18, and
a pharmaceutically acceptable carrier.

21. A method of inhibiting cell migration-inducing and hyaluronan-binding protein (CEMIP) signaling in a subject in need thereof, said method comprising:
Administering, to the subject in need of CEMIP inhibition, the pharmaceutical composition of claim 20, wherein said composition is administered in an amount effective to decrease CEMIP signaling in the subject relative to CEMIP signaling in the subject prior to said administering.

22. The method of claim 21, wherein said subject has a primary tumor.

23. The method of claim 22, wherein the primary tumor is selected from a breast tumor, lung tumor, melanoma, renal tumor, colorectal tumor, esophageal tumor, small intestine tumor, stomach tumor, bladder tumor, liver tumor, pancreatic tumor, or prostate tumor.

24. The method of claim 21, wherein the subject has an immune condition.

25. The method of claim 21, wherein the subject has an autoimmune condition.

26. A method of treating or inhibiting brain metastasis in a subject, said method comprising:
administering, to a subject having a primary tumor, a cell migration-inducing and hyaluronan-binding protein (CEMIP) antibody or binding fragment thereof in an amount effect to treat or prevent brain metastasis in the subject.

27. The method of claim 26, wherein the subject has a primary tumor selected from a breast tumor, lung tumor, melanoma, renal tumor, colorectal tumor, esophageal tumor, small intestine tumor, stomach tumor, bladder tumor, liver tumor, pancreatic tumor, or prostate tumor.

28. A method of treating an autoimmune condition in a subject, said method comprising:
administering, to a subject having an autoimmune condition, the pharmaceutical composition of claim 20, thereby treating the autoimmune condition in the subject.

29. The method of claim 28, wherein the autoimmune condition is an autoimmune form of arthritis.

30. A method of treating an inflammatory condition in a subject, said method comprising:
administering, to a subject having an inflammatory condition, the pharmaceutical composition of claim 20, thereby treating the inflammatory condition in the subject.

31. The method of claim 30, wherein the inflammatory condition is multiple sclerosis.

32. The method of claim 30, wherein the inflammatory condition is arthritis.


SEQUENCE LISTING (113 sequences)

SEQ ID NO: 1 - protein, 1361 aa, homo sapiens
MGAAGRQDFLFKAMLTISWLTLTCFPGATSTVAAGCPDQSPELQPWNPGHDQDHHVHIGQGKTLLLTSSATVYSIHISEGGKLVIKDHDEPIVLRTRHILIDNGGELHAGSALCPFQGNFTIILYGRADEGIQPDPYYGLKYIGVGKGGALELHGQKKLSWTFLNKTLHPGGMAEGGYFFERSWGHRGVIVHVIDPKSGTVIHSDRFDTYRSKKESERLVQYLNAVPDGRILSVAVNDEGSRNLDDMARKAMTKLGSKHFLHLGFRHPWSFLTVKGNPSSSVEDHIEYHGHRGSAAARVFKLFQTEHGEYFNVSLSSEWVQDVEWTEWFDHDKVSQTKGGEKISDLWKAHPGKICNRPIDIQATTMDGVNLSTEVVYKKGQDYRFACYDRGRACRSYRVRFLCGKPVRPKLTVTIDTNVNSTILNLEDNVQSWKPGDTLVIASTDYSMYQAEEFQVLPCRSCAPNQVKVAGKPMYLHIGEEIDGVDMRAEVGLLSRNIIVMGEMEDKCYPYRNHICNFFDFDTFGGHIKFALGFKAAHLEGTELKHMGQQLVGQYPIHFHLAGDVDERGGYDPPTYIRDLSIHHTFSRCVTVHGSNGLLIKDVVGYNSLGHCFFTEDGPEERNTFDHCLGLLVKSGTLLPSDRDSKMCKMITEDSYPGYIPKPRQDCNAVSTFWMANPNNNLINCAAAGSEETGFWFIFHHVPTGPSVGMYSPGYSEHIPLGKFYNNRAHSNYRAGMIIDNGVKTTEASAKDKRPFLSIISARYSPHQDADPLKPREPAIIRHFIAYKNQDHGAWLRGGDVWLDSCRFADNGIGLTLASGGTFPYDDGSKQEIKNSLFVGESGNVGTEMMDNRIWGPGGLDHSGRTLPIGQNFPIRGIQLYDGPINIQNCTFRKFVALEGRHTSALAFRLNNAWQSCPHNNVTGIAFEDVPITSRVFFGEPGPWFNQLDMDGDKTSVFHDVDGSVSEYPGSYLTKNDNWLVRHPDCINVPDWRGAICSGCYAQMYIQAYKTSNLRMKIIKNDFPSHPLYLEGALTRSTHYQQYQPVVTLQKGYTIHWDQTAPAELAIWLINFNKGDWIRVGLCYPRGTTFSILSDVHNRLLKQTSKTGVFVRTLQMDKVEQSYPGRSHYYWDEDSGLLFLKLKAQNEREKFAFCSMKGCERIKIKALIPKNAGVSDCTATAYPKFTERAVVDVPMPKKLFGSQLKTKDHFLEVKMESSKQHFFHLWNDFAYIEVDGKKYPSSEDGIQVVVIDGNQGRVVSHTSFRNSILQGIPWQLFNYVATIPDNSIVLMASKGRYVSRGPWTRVLEKLGADRGLKLKEQMAFVGFKGSFRPIWVTLDTEDHKAKIFQVVPIPVVKKKKL

SEQ ID NO: 2 - protein, 5 aa, Artificial
  CEMIP antibody heavy chain CDR
SFGMH

SEQ ID NO: 3 - protein, 5 aa, Artificial
  CEMIP antibody heavy chain CDR
SFGMH

SEQ ID NO: 4 - protein, 7 aa, Artificial
  CEMIP antibody heavy chain CDR
TSGMGVG

SEQ ID NO: 5 - protein, 5 aa, Artificial
  CEMIP antibody heavy chain CDR
AYTMN

SEQ ID NO: 6 - protein, 5 aa, Artificial
  CEMIP antibody heavy chain CDR
AYTMN

SEQ ID NO: 7 - protein, 5 aa, Artificial
  CEMIP antibody heavy chain CDR
DTYMH

SEQ ID NO: 8 - protein, 5 aa, Artificial
  CEMIP antibody heavy chain CDR
AYTMN

SEQ ID NO: 9 - protein, 17 aa, Artificial
  CEMIP antibody heavy chain CDR
YISSASNTIYYADTVKG

SEQ ID NO: 10 - protein, 17 aa, Artificial
  CEMIP antibody heavy chain CDR
YISSASNTIYYADTVKG

SEQ ID NO: 11 - protein, 16 aa, Artificial
  CEMIP antibody heavy chain CDR
HIWWNDEKYYNAALKS

SEQ ID NO: 12 - protein, 17 aa, Artificial
  CEMIP antibody heavy chain CDR
LINPYNGGTTYNQKFKG

SEQ ID NO: 13 - protein, 17 aa, Artificial
  CEMIP antibody heavy chain CDR
LINPYNGGTTYNQKFKG

SEQ ID NO: 14 - protein, 17 aa, Artificial
  CEMIP antibody heavy chain CDR
NIDPANGHTKYAPKFQG

SEQ ID NO: 15 - protein, 17 aa, Artificial
  CEMIP antibody heavy chain CDR
LINPYNGGTTYNQKFKG

SEQ ID NO: 16 - protein, 10 aa, Artificial
  CEMIP antibody heavy chain CDR
RDWDLYAMDY

SEQ ID NO: 17 - protein, 10 aa, Artificial
  CEMIP antibody heavy chain CDR
RNWDLYAMDY

SEQ ID NO: 18 - protein, 8 aa, Artificial
  CEMIP antibody heavy chain CDR
ITGAWFPY

SEQ ID NO: 19 - protein, 9 aa, Artificial
  CEMIP antibody heavy chain CDR
YDYGYAMDY

SEQ ID NO: 20 - protein, 9 aa, Artificial
  CEMIP antibody heavy chain CDR
YDYGYAMDY

SEQ ID NO: 21 - protein, 10 aa, Artificial
  CEMIP antibody heavy chain CDR
SNDYDVDFDY

SEQ ID NO: 22 - protein, 11 aa, Artificial
  CEMIP antibody heavy chain CDR
YYGGRGWYFDV

SEQ ID NO: 23 - protein, 11 aa, Artificial
  CEMIP antibody light chain CDR
KASQNVGTAVA

SEQ ID NO: 24 - protein, 11 aa, Artificial
  CEMIP antibody light chain CDR
KANQNVGTAVA

SEQ ID NO: 25 - protein, 11 aa, Artificial
  CEMIP antibody light chain CDR
RASQHISEYLH

SEQ ID NO: 26 - protein, 16 aa, Artificial
  CEMIP antibody light chain CDR
RSSQSIVHRNGNTYLE

SEQ ID NO: 27 - protein, 16 aa, Artificial
  CEMIP antibody light chain CDR
RSSQSIVHRSGNTYLE

SEQ ID NO: 28 - protein, 17 aa, Artificial
  CEMIP antibody light chain CDR
KSSQSLLNSRTRKNYLA

SEQ ID NO: 29 - protein, 15 aa, Artificial
  CEMIP antibody light chain CDR
RASKSVSTSGYSYVH

SEQ ID NO: 30 - protein, 7 aa, Artificial
  CEMIP antibody light chain CDR
SASNRHT

SEQ ID NO: 31 - protein, 7 aa, Artificial
  CEMIP antibody light chain CDR
STSNRDT

SEQ ID NO: 32 - protein, 7 aa, Artificial
  CEMIP antibody light chain CDR
YGSQSIS

SEQ ID NO: 33 - protein, 7 aa, Artificial
  CEMIP antibody light chain CDR
KVSNRFS

SEQ ID NO: 34 - protein, 7 aa, Artificial
  CEMIP antibody light chain CDR
KVSNRFS

SEQ ID NO: 35 - protein, 7 aa, Artificial
  CEMIP antibody light chain CDR
WASTRES

SEQ ID NO: 36 - protein, 7 aa, Artificial
  CEMIP antibody light chain CDR
LASNLES

SEQ ID NO: 37 - protein, 8 aa, Artificial
  CEMIP antibody light chain CDR
QQYSSSPT

SEQ ID NO: 38 - protein, 8 aa, Artificial
  CEMIP antibody light chain CDR
QQYRNYPT

SEQ ID NO: 39 - protein, 9 aa, Artificial
  CEMIP antibody light chain CDR
QNGHSFPYT

SEQ ID NO: 40 - protein, 9 aa, Artificial
  CEMIP antibody light chain CDR
FQGSHVPWT

SEQ ID NO: 41 - protein, 9 aa, Artificial
  CEMIP antibody light chain CDR
FQGSHVPWT

SEQ ID NO: 42 - protein, 8 aa, Artificial
  CEMIP antibody light chain CDR
KQSYNLYT

SEQ ID NO: 43 - protein, 9 aa, Artificial
  CEMIP antibody light chain CDR
QNSRELPYT

SEQ ID NO: 44 - protein, 119 aa, Artificial
  CEMIP VH domain
DVQLVESGGGLVQPGGSRKLSCAASGFAFSSFGMHWVRQAPERGLEWVAYISSASNTIYYADTVKGRFTISRDNPKSTLFLQMTSLRSEDTAIYYCAKRDWDLYAMDYWGQGTSVTVSS

SEQ ID NO: 45 - protein, 106 aa, Artificial
  CEMIP VL domain
DIVMTQSQKFLSTSVGDRVTITCKASQNVGTAVAWYQQKPGQSPELLIYSASNRHTGVPARFTGSGSGTDFTLTITNMQSEDLADYFCQQYSSSPTFGGGTKLESK

SEQ ID NO: 46 - protein, 119 aa, Artificial
  CEMIP VH domain
DVQLVESGGGLVQPGGSRKLSCAASGFPFSSFGMHWVRQAPDKGLEWVAYISSASNTIYYADTVKGRFTVSRDNPKNTLFLQMTSLRSEDTAIYYCTKRNWDLYAMDYWGQGTSVTVSS

SEQ ID NO: 47 - protein, 106 aa, Artificial
  CEMIP VL domain
DIVMTQSQKFMSTSVGDRVSITCKANQNVGTAVAWYQQKPGQSPKLLIYSTSNRDTGVPDRFTGSGSGTDFTLTINYIQSEDLADYFCQQYRNYPTFGGGTKLEIK

SEQ ID NO: 48 - protein, 118 aa, Artificial
  CEMIP VH domain
QVTLKESGPGILQPSQTLSLTCSFSGFSLSTSGMGVGWIRQPSGKGLEWLAHIWWNDEKYYNAALKSRLTISKDTSKNQVFLKIASVDAADTATYYCARITGAWFPYWGQGTLVTVSA

SEQ ID NO: 49 - protein, 107 aa, Artificial
  CEMIP VL domain
DIVMTQSPATLSVTPGDRVSLSCRASQHISEYLHWYQQKSHESPRLLIKYGSQSISGIPSRFSGSGSGSDFTLNINSVEPEDVGVYYCQNGHSFPYTFGGGTKLEIK

SEQ ID NO: 50 - protein, 118 aa, Artificial
  CEMIP VH domain
EVQLQQSGPELVKPGASMKISCKASGYSFTAYTMNWVKQSHGENLEWIGLINPYNGGTTYNQKFKGKATLTVDKSSSTAYMELLSLTSEDSAVYYCASYDYGYAMDYWGQGTSVTVSS

SEQ ID NO: 51 - protein, 112 aa, Artificial
  CEMIP VL domain
DVLMTQTPLSLPVSLGDQASISCRSSQSIVHRNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 52 - protein, 118 aa, Artificial
  CEMIP VH domain
EVQLQQSGPELVKPGVSMKISCKASGYSFTAYTMNWVKQSHGENLEWIGLINPYNGGTTYNQKFKGKATLTVDKSSSTAYMELLSLTSEDSAVYYCASYDYGYAMDYWGQGTSVTVSS

SEQ ID NO: 53 - protein, 112 aa, Artificial
  CEMIP VL domain
DVLMTQTPLSLPVSLGDQASISCRSSQSIVHRSGNTYLEWYLQKPGQSPKLLIYKVSNRFSGVPDRFSGSGSGTDFTLKISRVEAEDLGVYYCFQGSHVPWTFGGGTKLEIK

SEQ ID NO: 54 - protein, 119 aa, Artificial
  CEMIP VH domain
EVQLQQSGAELVKPGASVKLSCTASGFNIKDTYMHWVKQRPEQGLIWIGNIDPANGHTKYAPKFQGKATITADTSSNTAYLQLSSLTSEDTAVYYCARSNDYDVDFDYWGQGTTLTVSS

SEQ ID NO: 55 - protein, 112 aa, Artificial
  CEMIP VL domain
DIVMSQSPSSLAVSAGEKVTMSCKSSQSLLNSRTRKNYLAWYQQKPGQSPKLLIYWASTRESGVPDRFTGSGSGTDFSLTISSVQAEDLAVYYCKQSYNLYTFGGGTKLEIK

SEQ ID NO: 56 - protein, 120 aa, Artificial
  CEMIP VH domain
EVHLQQSGPELVKPGASMKISCKASGYSFTAYTMNWVKQSHGKNLEWIGLINPYNGGTTYNQKFKGKATLTVDKSSSTAYMELLSLTSEDSAVYYCASYYGGRGWYFDVWGAGTSVTVSS

SEQ ID NO: 57 - protein, 111 aa, Artificial
  CEMIP VL domain
DIVLTQSPASLAVSLGQRATISCRASKSVSTSGYSYVHWYQQKPGQPPKLLIYLASNLESGVPARFSGSGSGTDFTLNIHPVEEEDAATYYCQNSRELPYTFGGGTKLEMK

SEQ ID NO: 58 - DNA, 357 bp, Artificial
  CEMIP VH domain
gatgtgcagctggtggagtctgggggaggcttagtgcagcctggagggtcccggaaactctcctgtgcagcctctggattcgctttcagtagctttggaatgcactgggttcgtcaggctccagagagggggctggagtgggtcgcatacattagtagtgccagcaataccatctactatgcagacacagtgaagggccgattcaccatctccagagacaatcccaagagcaccctgttcctgcaaatgaccagtctaaggtctgaggacacggccatttattactgtgcaaagcgagactgggacctctatgctatggactactggggtcaaggaacctcagtcaccgtctcgtca

SEQ ID NO: 59 - DNA, 318 bp, Artificial
  CEMIP VL domain
gacattgtgatgacccagtctcaaaaattcttgtccacatcagtgggagacagggtcaccatcacctgcaaggccagtcagaatgtgggaactgctgttgcctggtatcaacagaaaccagggcaatctcctgaacttctgatttactcggcatccaatcggcacactggagtccctgctcgcttcacaggcagtggatctgggacagatttcactctcaccatcaccaatatgcagtctgaagacctggcagattatttctgccagcaatatagtagctcgccgacattcggtggaggcaccaagttggaatccaaa

SEQ ID NO: 60 - DNA, 357 bp, Artificial
  CEMIP VH domain
gatgtgcagctggtggagtctgggggaggcttagtgcagcctggagggtcccggaaactctcctgtgcagcctctggattccctttcagtagctttggaatgcactgggttcgtcaggctccagacaagggactggagtgggtcgcatacattagtagtgccagtaataccatctactatgctgacacagtgaagggccgattcaccgtctccagagacaatcccaagaacaccctgttcctgcaaatgaccagtctaaggtctgaggacacggccatttattactgtacaaagcgaaattgggacctctatgctatggactactggggtcaaggaacctcagtcaccgtctcctca

SEQ ID NO: 61 - DNA, 318 bp, Artificial
  CEMIP VL domain
gacattgtgatgacccagtctcaaaaattcatgtccacatcagtgggagacagggtcagcatcacctgcaaggccaatcagaatgtgggtactgctgtagcctggtatcaacagaaaccaggacaatctcctaaactactgatttattcgacatccaatcgggacactggagtccctgaccgcttcacaggcagtggatctgggacagatttcactctcaccatcaactatattcaatctgaagacctggcagattatttctgccagcaatatagaaattatccgacgttcggtggaggcaccaagctggaaatcaaa

SEQ ID NO: 62 - DNA, 354 bp, Artificial
  CEMIP VH domain
caggttactctgaaagagtctggccctgggatattgcagccctcccagaccctcagtctgacttgttctttctctgggttttcactgagcacttctggtatgggtgtaggctggattcgtcagccttcagggaagggtctggagtggctggcacacatttggtggaatgatgagaaatactataacgcagccctgaagagccggctcacaatctccaaggatacctccaaaaaccaggttttcctcaagatcgccagtgtggacgctgcagatactgccacatactactgtgctcgcatcactggtgcctggtttccttattggggccaagggactctggtcactgtctctgca

SEQ ID NO: 63 - DNA, 321 bp, Artificial
  CEMIP VL domain
gacattgtgatgactcagtctccagccaccctgtctgtgactccaggagatagagtctctctttcctgcagggccagtcagcatattagcgaatacttacactggtatcaacaaaaatcacacgagtctccaaggcttctcatcaaatatggttcccaatccatctctgggatcccctccaggttcagtggcagtggatcagggtcagatttcactctcaatatcaacagtgtggaacctgaagatgttggagtgtattactgtcaaaatggtcacagctttccgtacacgttcggaggggggaccaagctggaaataaaa

SEQ ID NO: 64 - DNA, 354 bp, Artificial
  CEMIP VH domain
gaggtccagctgcaacagtctggacctgagctggtgaagcctggagcctcaatgaagatatcttgcaaggcttctggttactcattcactgcctacaccatgaactgggtgaagcagagccatggagagaaccttgagtggattggacttattaatccttataatggtggtactacctacaaccagaagttcaagggcaaggccacattaactgtagacaagtcatccagcacagcctacatggagctcctcagtctgacatctgaggactctgcagtctattactgtgcatcatatgattacggctatgctatggactactggggtcaaggaacctcagtcaccgtctcctca

SEQ ID NO: 65 - DNA, 336 bp, Artificial
  CEMIP VL domain
gatgttttgatgacccaaactccactctccctgcctgtcagtcttggagatcaagcctccatctcttgcagatctagtcagagcattgtacatagaaatggaaacacctatctagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 66 - DNA, 354 bp, Artificial
  CEMIP VH domain
gaggtccagctgcaacagtctggacctgagctggtgaagcctggagtttcaatgaagatatcctgcaaggcttctggttactcattcactgcctacactatgaattgggtgaagcagagtcatggagagaaccttgagtggattggacttattaatccttataatggtggtactacctacaaccagaagttcaagggcaaggccacattaactgtagacaagtcatccagcacagcctacatggagctcctcagtctgacatctgaggactctgcagtctattactgtgcatcatatgattacggctatgctatggactactggggtcaaggaacctcagtcaccgtctcctca

SEQ ID NO: 67 - DNA, 336 bp, Artificial
  CEMIP VL domain
gatgttttgatgacccaaactccactctccctgcctgtcagtcttggagatcaagcctccatctcttgcagatctagtcagagcattgtacatcgtagtggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagttggaaatcaaa

SEQ ID NO: 68 - DNA, 357 bp, Artificial
  CEMIP VH domain
gaggttcaactgcagcagtctggggcagagcttgtgaagccaggggcctcagtcaagttgtcctgcacagcttctggcttcaacattaaagacacctatatgcactgggtgaaacagaggcctgagcagggcctgatttggattggaaacattgatcctgcgaatggtcatactaaatatgccccgaagttccagggcaaggccactatcacagcagacacatcctccaacacagcctacctgcagctcagcagcctgacatctgaggacactgccgtctattactgtgctagatctaatgattacgacgtcgactttgactactggggccaaggcaccactctcacagtctcctca

SEQ ID NO: 69 - DNA, 336 bp, Artificial
  CEMIP VL domain
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcagtctcaccatcagcagtgtgcaggctgaagacctggcagtttattactgcaagcaatcttataatctatacacgttcggaggggggaccaagctggaaataaaa

SEQ ID NO: 70 - DNA, 360 bp, Artificial
  CEMIP VH domain
gaggtccacctgcaacagtctggacctgagctggtgaagcctggagcttcaatgaagatatcctgcaaggcttctggttactccttcactgcctacaccatgaactgggtgaaacagagccatggaaagaaccttgagtggattggacttattaatccttacaatggtggtactacctacaaccagaaattcaagggcaaggccacattaactgtagacaagtcatccagcacagcctacatggagctcctcagtctgacatctgaggactctgcagtctattactgtgcttcttactacggtggtaggggctggtacttcgatgtctggggcgcagggacctcggtcaccgtctcctca

SEQ ID NO: 71 - DNA, 333 bp, Artificial
  CEMIP VL domain
gacattgtgctgacacagtctcctgcttccttagctgtatctctggggcagagggccaccatctcatgcagggccagcaaaagtgtcagtacatctggctatagttatgttcactggtaccaacagaaaccaggacagccacccaaactcctcatctatcttgcatccaacctagaatctggggtccctgccaggttcagtggcagtgggtctgggacagacttcaccctcaacatccatcctgtggaggaggaagatgctgcaacctattactgtcagaatagtagggaacttccgtacacgttcggaggggggaccaagctggaaatgaaa

SEQ ID NO: 72 - protein, 330 aa, Artificial
  CEMIP antibody
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 73 - protein, 107 aa, Artificial
  CEMIP antibody
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 74 - protein, 330 aa, Artificial; identical to SEQ ID NO: 72
  CEMIP antibody

SEQ ID NO: 75 - protein, 107 aa, Artificial; identical to SEQ ID NO: 73
  CEMIP antibody

SEQ ID NO: 76 - protein, 330 aa, Artificial
  CEMIP antibody
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 77 - protein, 107 aa, Artificial; identical to SEQ ID NO: 73
  CEMIP antibody

SEQ ID NO: 78 - protein, 324 aa, Artificial
  CEMIP antibody
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 79 - protein, 107 aa, Artificial; identical to SEQ ID NO: 73
  CEMIP antibody

SEQ ID NO: 80 - protein, 324 aa, Artificial; identical to SEQ ID NO: 78
  CEMIP antibody

SEQ ID NO: 81 - protein, 107 aa, Artificial; identical to SEQ ID NO: 73
  CEMIP antibody

SEQ ID NO: 82 - protein, 330 aa, Artificial; identical to SEQ ID NO: 72
  CEMIP antibody

SEQ ID NO: 83 - protein, 107 aa, Artificial; identical to SEQ ID NO: 73
  CEMIP antibody

SEQ ID NO: 84 - protein, 324 aa, Artificial; identical to SEQ ID NO: 78
  CEMIP antibody

SEQ ID NO: 85 - protein, 107 aa, Artificial; identical to SEQ ID NO: 73
  CEMIP antibody

SEQ ID NO: 86 - DNA, 990 bp, Artificial
  CEMIP antibody
gccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 87 - DNA, 57 bp, Artificial
  CEMIP antibody
atggactccaggctcaatttagttttccttgtccttattttaaaaggtgtccaatgt

SEQ ID NO: 88 - DNA, 321 bp, Artificial
  CEMIP antibody
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 89 - DNA, 60 bp, Artificial
  CEMIP antibody
atggagtctcatactcaggcctttgtattcgcgtttctctggttgtctggtgttgatgga

SEQ ID NO: 90 - DNA, 990 bp, Artificial; identical to SEQ ID NO: 86
  CEMIP antibody

SEQ ID NO: 91 - DNA, 57 bp, Artificial
  CEMIP antibody
atggactccaggctcaatttagttttccttgtccttattttaaaaggtgtccagtgt

SEQ ID NO: 92 - DNA, 321 bp, Artificial; identical to SEQ ID NO: 88
  CEMIP antibody

SEQ ID NO: 93 - DNA, 60 bp, Artificial
  CEMIP antibody
atggagtctcgtactcaggcctttgtaatcgcgtttctctggatgtctggtattgatgga

SEQ ID NO: 94 - DNA, 990 bp, Artificial
  CEMIP antibody
gccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagtcccatggtcacatgtgtggtggtggatgtgagcgaggatgacccagatgtccagatcagctggttcgtgaacaacgtggaagtactcacagctcagacacaaacccatagagaggattacaacagtactctccgggtggtcagtgccctccccatccagcaccaggactggatgagtggcaaggagttcaaatgcaaggtcaacaacaaagacctcccagcacccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 95 - DNA, 57 bp, Artificial
  CEMIP antibody
atgggcagacttacttcttcattcttgctactgattgtccctgcatatgtcctgtcc

SEQ ID NO: 96 - DNA, 321 bp, Artificial; identical to SEQ ID NO: 88
  CEMIP antibody

SEQ ID NO: 97 - DNA, 60 bp, Artificial
  CEMIP antibody
atggtgtccacttctcagctccttggacttttgcttttctggacttcagcctccagatgt

SEQ ID NO: 98 - DNA, 972 bp, Artificial
  CEMIP antibody
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 99 - DNA, 57 bp, Artificial
  CEMIP antibody
atgggatggagctggatctttctcttcctcctgtcaggaactgcaggtgtccactct

SEQ ID NO: 100 - DNA, 321 bp, Artificial; identical to SEQ ID NO: 88
  CEMIP antibody

SEQ ID NO: 101 - DNA, 57 bp, Artificial
  CEMIP antibody
atgaagttgcctgttaggctgttggtgctgatgttctggattcctgcttccagtagt

SEQ ID NO: 102 - DNA, 972 bp, Artificial; identical to SEQ ID NO: 98
  CEMIP antibody

SEQ ID NO: 103 - DNA, 57 bp, Artificial; identical to SEQ ID NO: 99
  CEMIP antibody

SEQ ID NO: 104 - DNA, 321 bp, Artificial; identical to SEQ ID NO: 88
  CEMIP antibody

SEQ ID NO: 105 - DNA, 57 bp, Artificial
  CEMIP antibody
atgaagttgcctgttaggctgttggtgctgatgttctggattcctgcttccagcagt

SEQ ID NO: 106 - DNA, 990 bp, Artificial; identical to SEQ ID NO: 86
  CEMIP antibody

SEQ ID NO: 107 - DNA, 57 bp, Artificial
  CEMIP antibody
atgaaatgcagctgggttatcttcttcctgatggcagtggttacaggggtcaattca

SEQ ID NO: 108 - DNA, 321 bp, Artificial; identical to SEQ ID NO: 88
  CEMIP antibody

SEQ ID NO: 109 - DNA, 60 bp, Artificial
  CEMIP antibody
atggattcacgggcccaggttcttatattgctgctgcaatgggtatctggtacctgtggg

SEQ ID NO: 110 - DNA, 972 bp, Artificial; identical to SEQ ID NO: 98
  CEMIP antibody

SEQ ID NO: 111 - DNA, 57 bp, Artificial; identical to SEQ ID NO: 99
  CEMIP antibody

SEQ ID NO: 112 - DNA, 321 bp, Artificial; identical to SEQ ID NO: 88
  CEMIP antibody

SEQ ID NO: 113 - DNA, 60 bp, Artificial
  CEMIP antibody
atggagacagacacactcctgttatgggtactgctgctctgggttccaggttccactggt